(19) United States
(12) Patent Application Publication
(10) Pub. No.: US 2026/0248914 A1
(43) Pub. Date: Aug. 27, 2026

(54) MULTIFUNCTIONAL CELLS TRANSIENTLY EXPRESSING AN IMMUNE RECEPTOR AND ONE OR MORE CYTOKINES, THEIR USE AND METHODS FOR THEIR PRODUCTION

(71) Applicant: BioNTech SE, Mainz (DE)

(72) Inventors: Ugur SAHIN, Mainz (DE); Steve PASCOLO, Schlieren (CH); Tobias WEISS, Zurich (CH)

(21) Appl. No.: 18/995,574

(22) PCT Filed: Jul. 20, 2023
(86) PCT No.: PCT/EP2023/070227

§ 371 Date: Jan. 16, 2025

(30) Foreign Application Priority Data
Jul. 21, 2022 (WO) PCT/EP2022/070564

(51) Int. Cl. A61K 40/11; A61K 40/31; A61K 40/35; A61K 40/42; A61P 35/00; C07K 14/54; C07K 14/56; C07K 16/28; C12N 5/0783

(52) U.S. Cl. CPC A61K 40/11; A61K 40/31; A61K 40/35; A61K 40/4202; A61P 35/00; C07K 14/5434; C07K 14/56; C07K 16/2851; C12N 5/0638; C12N 2510/00

(57) ABSTRACT

Disclosed herein is a multifunctional cell recombinantly expressing an immune receptor and two or more cytokines, wherein the cell comprises one or more exogenous RNA molecules encoding the immune receptor and the two or more cytokines. The expression in the multifunctional cell is transient. Also disclosed herein are methods of using such a multifunctional cell in the treatment of diseases and disorders characterized by expression of a protein or antigen that is able to be bound by the immune receptor, such as cancer, for example glioblastoma. Also disclosed herein are methods for the production of such a multifunctional cell.

### TECHNICAL FIELD

The present disclosure provides a multifunctional cell, in particular an immune cell, transiently expressing an immune receptor and one or more cytokines, preferably two or more cytokines, wherein the cell comprises one or more RNA molecules encoding the receptor and one or more, preferably two or more, cytokines. The multifunctional cell can be produced by transfecting the one or more RNA molecules into the cell, such that the cell translates the RNA molecules and the immune receptor and the one (two) or more cytokines encoded by the RNA molecules are transiently expressed by the cell. The multifunctional cell is useful in the treatment of diseases and disorders which are characterized by the expression of a protein that is bound by the immune receptor, for example, treating cancer where the immune receptor binds to an antigen expressed by the cancer cell.

### BACKGROUND

Apart from their well-known ability to encode biologically active proteins, nucleic acids such as DNA and mRNA have other remarkable properties that make them attractive therapeutic agents. Nucleic acid-based therapeutics are easy to manufacture and relatively inexpensive.

Generally, in vivo DNA is more stable than RNA, but has some potential safety risks such as the induction of anti-DNA antibodies and the integration of the transgene into the host genome.

The use of RNA to deliver foreign genetic information into target cells offers an attractive alternative to DNA. The advantages of mRNA include transient expression and non-transforming character. mRNA does not require nucleus infiltration for expression and moreover cannot integrate into the host genome, thereby eliminating the risk of oncogenesis. Glioblastoma is the most common and most aggressive primary brain tumor in adults (Ostrom Q T et al., Neuro-oncology 2020; 22 (Supplement_1): iv1-iv96). It is an inevitably fatal disease with an urgent need for more effective therapies than the current standard of care comprising surgery followed by radiochemotherapy with temozolomide (Weller M et al., Nature reviews Clinical oncology 2021; 18 (3): 170-86). Chimeric antigen receptor (CAR) T cell therapy has led to impressive clinical responses in hematological malignancies (Maude S L et al., The New England Journal of Medicine 2018; 378 (5): 439-48, Park J H et al., The New England Journal of Medicine 2018; 378 (5): 449-59) and is also explored against glioblastoma. However, apart from a single case report (Brown C E et al., The New England Journal of Medicine 2016; 375 (26): 2561-9), the anti-tumor activity of CAR T cells in patients with glioblastoma has only been modest (Ahmed N et al., JAMA Oncology 2017; 3 (8): 1094-101, O'Rourke D M et al., Science Translational Medicine 2017; 9 (399)). Challenges in current clinical trials with CAR T cells against glioblastoma are heterogeneously expressed single-target antigens such as epidermal growth factor receptor variant III (EGFRVIII), epidermal growth factor 2 (Her2), or interleukin-13 receptor alpha 2 (IL13Rα2) as well as an immunosuppressive microenvironment averting CAR T cell activity. Multi-targeting strategies based on unconventional CAR designs such as natural killer group 2D (NKG2D) receptor-based CAR T cells (Weiss T et al., Cancer Res 2018; 78 (4): 1031-43, Yang D et al., Journal for Immunotherapy of Cancer 2019; 7 (1): 171) or tandem-CARs (Ponterio E et al., Frontiers in Immunology 2020; 11:565631) based on multiple single-chain variable fragments (scFVs) as well as cytokine-armored CAR constructs that co-express a CAR and proinflammatory cytokines such as interleukin 12 (IL12) (Yeku O O et al., Sci Rep 2017; 7 (1): 10541-41) or type I interferons (IFN) (Katlinski K V et al., Cancer Cell 2017; 31 (2): 194-207) are promising emerging strategies to overcome these challenges.

However, the majority of CAR T cell approaches against glioblastoma use retroviral vectors or non-viral transposon-transposase systems to stably integrate transgenes encoding the CAR and potentially also cytokines. These enable long-term expression but have limitations and safety concerns such as a limited transgene capacity, long production processes with the risk of treatment delays, the risk of genomic alterations that could lead to malignant transformation of T cell clones, and persistent CAR and/or cytokine expression with the risk for off-tumor toxicities (Lamers C H et al., J Clin Oncol 2006; 24 (13): e20-2, Neelapu S S et al., Nature Reviews Clinical Oncology 2018; 15 (1): 47-62). Consequently, regulatory hurdles associated with genetically engineered cell therapies are strict, which delays the clinical translation of innovative CAR T cell strategies against glioblastoma (Rafiq S et al., Nature Reviews Clinical Oncology 2020; 17 (3): 147-67).

Many solid tumors have the problem that they have an immunosuppressive microenvironment. Current treatments, in particular vector-based CAR T cell strategies have limited transgene capacity, require lengthy manufacturing processes. Integration into the genome and continuous transgene expression pose the risk for malignant transformation and uncontrollable off-tumor toxicities.

Therefore, there is a need to improve existing treatments of cancer, in particular solid tumors, in particular with a immunosuppressive environment. Preferably these treatments should be efficient and and safe, in particular they should provide reduced production time and reduce the risk of genomic alterations and/or off-tumor toxicities.

### SUMMARY

The inventors have surprisingly found that it is possible to provide multifunctional cells that offer efficient and safe treatment of diseases or disorders, in particular cancer, solely by the transfection of exogenous RNA molecules encoding an immune receptor and one or more cytokines, preferably two or more cytokines, into the multifunctional cells for recombinant, transient expression.

In an aspect of the disclosure, a multifunctional cell is provided that transiently expresses an immune receptor and two or more cytokines, wherein the cell comprises one or more exogenous RNA molecules encoding the immune receptor and the two or more cytokines.

Since the one or more RNA molecules does not stably integrate into the genome of the multifunctional cell, the recombinant expression of the encoded immune receptor and cytokines occurs only transiently. Thus, in the context of the present disclosure, the terms recombinant expression is interchangeable with transient expression.

In an embodiment, the multifunctional cell does not contain any functional exogenous DNA molecules encoding the immune receptor and/or the two or more cytokines that can be transcribed to express the immune receptor and/or the two or more cytokines. Preferably, the multifunctional cell does not comprise any exogenous DNA encoding the immune receptor and/or the two or more cytokines.

In an embodiment, the one or more exogenous RNA molecules can encode three or more cytokines. For example, 3, 4, 5, 6, 7, 8, 9, 10 or more different cytokines can be encoded and can be expressed. In some cases where the wild-type cytokine is comprised of individual chains, each individual chain optionally can be considered as one cytokine.

In an embodiment, the immune receptor can be encoded by a separate exogenous RNA molecule from the two or more cytokines. In an embodiment, each of the two or more cytokines can be encoded by separate exogenous RNA molecules. In an embodiment, the immune receptor and the two or more cytokines can be encoded by a single RNA molecule. In an embodiment, the immune receptor and each cytokine can be encoded by separate exogenous RNA molecules.

The exogenous RNA molecules are preferably not produced by the cell, except where the RNA molecule is a replicable RNA molecule, e.g., a replicon. For example, the exogenous RNA is not transcribed from DNA in the multifunctional cell. Thus, in an embodiment, the one or more exogenous RNA molecules can be an in vitro transcribed RNA molecule. In an embodiment, the one or more exogenous RNA molecules can be a synthetic RNA molecule. In an embodiment, the one or more exogenous RNA molecules is not produced by transcription from DNA present in the multifunctional cell.

In an embodiment, the multifunctional cell or the cell from which the multifunctional cell is derived, e.g., by the methods of production of such multifunctional cell disclosed herein, can be obtained from peripheral blood, bone marrow, spleen, tumor infiltrating lymphocytes, from a cell line or cell bank. In an embodiment, the cell can be a mononuclear cell, a peripheral blood mononuclear cell, a bone marrow cell, a lymphocyte, a splenocyte, a B-cell, a natural killer cell (NK cell) or a T-cell. In an embodiment, the cell can be a cytotoxic cell, for example a cytotoxic T cell or cytotoxic T lymphocyte. In an embodiment, the cell can be a CD8+ T cell.

In an embodiment, the immune receptor can be a chimeric antigen receptor (CAR) or a T cell receptor or a chimeric immune receptor. The immune receptor can be designed to target a particular protein or antigen that is expressed by a cell involved the disease or disorder to be treated according to the methods of treatment disclosed herein. The disease or disorder can be cancer or a disease or disorder caused by a pathogenic organism. In a preferred embodiment, the protein or antigen is expressed by cancer or tumor cells, for example, glioblastoma cells. For example, the immune receptor can bind to the NKG2D receptor.

In an embodiment, the two or more cytokines can be selected from two or more of the following cytokines: interleukin-2, interleukin-7, interleukin-10, interleukin-12, interleukin-15, interleukin-18 and interferon-α (IFN-α) or a subtype of, for example, IFN-α1, IFN-α2, IFN-α8, IFN-α10, IFN-α14 or IFN-α21. In a preferred embodiment, the two or more cytokines can be interleukin-12 and interferon-α2. Also encompassed in the disclosure herein is that the cytokine is a functionally equivalent molecule, such as a fragment or variant of the cytokine, or to other molecules which can activate/trigger the receptor for the relevant cytokine.

In an embodiment, one or more exogenous RNA molecules can be a linear or circular RNA molecule, such as a linear mRNA or a circular mRNA. In an embodiment, the one or more RNA molecules can be a non-replicable or a replicable RNA molecule via cis- or trans-replication. In an embodiment, the one or more exogenous RNA molecules can be a mixture of the foregoing different formats. For example, the RNA molecule encoding the immune receptor can be a replicable RNA molecule and the RNA molecule(s) encoding the two or more cytokines can be non-replicable RNA molecule(s). In another example, each exogenous RNA molecule is a linear non-replicable mRNA.

In another aspect of the present disclosure a multifunctional cell transiently expressing an immune receptor and two or more cytokines is provided for use in a method of treating a disease or disorder in a patient characterized by the expression of an antigen to which the immune receptor binds, wherein the cell comprises one or more exogenous RNA molecules encoding the immune receptor and the two or more cytokines, said method comprising administering the multifunctional cell to the patient, preferably wherein the patient is a human.

In an embodiment, the multifunctional cell does not contain any functional exogenous DNA molecules encoding the immune receptor and/or the two or more cytokines that can be transcribed to express the immune receptor and/or the two or more cytokines. Preferably, the multifunctional cell does not comprise any exogenous DNA encoding the immune receptor and/or the two or more cytokines.

In an embodiment, the one or more exogenous RNA molecules can encode three or more cytokines. For example, 3, 4, 5, 6, 7, 8, 9, 10 or more different cytokines can be encoded and can be expressed. In some cases where the wild-type cytokine is comprised of individual chains, each individual chain optionally can be considered as one cytokine.

In an embodiment, the immune receptor can be encoded by a separate exogenous RNA molecule from the two or more cytokines. In an embodiment, each of the two or more cytokines can be encoded by separate exogenous RNA molecules. In an embodiment, the immune receptor and the two or more cytokines can be encoded by a single RNA molecule. In an embodiment, the immune receptor and each cytokine can be encoded by separate exogenous RNA molecules.

The exogenous RNA molecules are preferably not produced by the cell, except where the RNA molecule is a replicable RNA molecule, e.g., a replicon. For example, the exogenous RNA is not transcribed from DNA in the multifunctional cell. Thus, in an embodiment, the one or more exogenous RNA molecules can be an in vitro transcribed RNA molecule. In an embodiment, the one or more exogenous RNA molecules can be a synthetic RNA molecule. In an embodiment, the one or more exogenous RNA molecules is not produced by transcription from DNA present in the multifunctional cell.

In an embodiment, the multifunctional cell or the cell from which the multifunctional cell is derived, e.g., by the methods of production of such multifunctional cell disclosed herein, can be obtained from peripheral blood, bone marrow, spleen, tumor infiltrating lymphocytes, from a cell line or cell bank. In an embodiment, the cell can be a mononuclear cell, a peripheral blood mononuclear cell, a bone marrow cell, a lymphocyte, a splenocyte, a B-cell, a natural killer cell (NK cell) or a T-cell. In an embodiment, the cell can be a cytotoxic cell, for example a cytotoxic T cell or cytotoxic T lymphocyte. In an embodiment, the cell can be a CD8+ T cell.

In an embodiment, the immune receptor can be a chimeric antigen receptor (CAR) or a T cell receptor or a chimeric immune receptor. The immune receptor can be designed to target a particular protein or antigen that is expressed by a cell involved the disease or disorder to be treated according to the methods of treatment disclosed herein. The disease or disorder can be cancer or a disease or disorder caused by a pathogenic organism. In a preferred embodiment, the protein or antigen is expressed by cancer or tumor cells, for example, glioblastoma cells. For example, the immune receptor can bind to the NKG2D receptor.

In an embodiment, the two or more cytokines can be selected from two or more of the following cytokines: interleukin-2, interleukin-7, interleukin-10, interleukin-12, interleukin-15, interleukin-18 and interferon-α (IFN-α) or a subtype of, for example, IFN-α1, IFN-α2, IFN-α8, IFN-α10, IFN-α14 or IFN-α21. In a preferred embodiment, the two or more cytokines can be interleukin-12 and interferon-α2. Also encompassed in the disclosure herein is that the cytokine is a functionally equivalent molecule, such as a fragment or variant of the cytokine, or to other molecules which can activate/trigger the receptor for the relevant cytokine.

In an embodiment, one or more exogenous RNA molecules can be a linear or circular RNA molecule, such as a linear mRNA or a circular mRNA. In an embodiment, the one or more RNA molecules can be a non-replicable or a replicable RNA molecule via cis- or trans-replication. In an embodiment, the one or more exogenous RNA molecules can be a mixture of the foregoing different formats. For example, the RNA molecule encoding the immune receptor can be a replicable RNA molecule and the RNA molecule(s) encoding the two or more cytokines can be non-replicable RNA molecule(s). In another example, each exogenous RNA molecule is a linear non-replicable mRNA.

In an embodiment, the multifunctional cell administered to the patient can be autologous or allogeneic or xenogeneic to the patient.

In an embodiment, the multifunctional cell can comprise a further genetic modification. For example, the genetic modification comprises (i) disrupting the expression of the endogenous T cell receptor (TCR) such that the TCR is expressed at reduced levels and/or (ii) disrupting the expression of the HLA/MHC complex such that the complex is expressed on the cell surface at reduced levels. In an embodiment, the reduced level of expression is no more than 35%, 30%, 25%, 20%, 15%, 10%, 5%, 4%, 3%, 2%, or 1% of the wild-type level of expression. In an embodiment, the endogenous TCR and/or the HLA/MHC complex is not detectably expressed on the surface of the immune cell. In an embodiment, the expression of the endogenous TCR and/or the HLA/MHC complex on the cell surface can be determined using a FACS assay.

In an aspect, the present disclosure provides a cytotoxic T cell expressing a chimeric antigen receptor (CAR) capable of binding to a NKG2D ligand, interleukin-12, and interferon-α2 for use in a method of treating glioblastoma, wherein the cytotoxic T cell comprises three exogenous mRNA molecules, the first encoding the CAR, the second encoding interleukin-12, and the third encoding interferon-α2, preferably wherein the cytotoxic T cell does not comprise any exogenous DNA sequences encoding the CAR, interleukin-12 and/or interferon-α2.

In an aspect, the present disclosure provides a method for producing a multifunctional cell expressing an immune receptor and two or more cytokines, said method comprising transfecting a mononuclear cell with one or more exogenous RNA molecules encoding the immune receptor and the two or more cytokines to produce a multifunctional cell expressing the immune receptor and the two or more cytokines. In an embodiment, the method is carried out in vitro, ex vivo or in vivo.

In an embodiment, one or more exogenous RNA molecules can be a linear or circular RNA molecule, such as a linear mRNA or a circular mRNA. In an embodiment, the one or more RNA molecules can be a non-replicable or a replicable RNA molecule via cis- or trans-replication. In an embodiment, the one or more exogenous RNA molecules can be a mixture of the foregoing different formats. For example, the RNA molecule encoding the immune receptor can be a replicable RNA molecule and the RNA molecule(s) encoding the two or more cytokines can be non-replicable RNA molecule(s). In another example, each exogenous RNA molecule is a linear non-replicable mRNA. In an embodiment, wherein the one or more exogenous RNA molecules can be complexed with a lipid particle or is complexed with a polymer, such as a protein.

In an embodiment, the transfection of the one or more exogenous RNA molecules can be by a method selected from the group consisting of electroporation, lipid-mediated transfection, calcium phosphate transfection, targeted liposomes, polymer-mediated transfection, particle mediated delivery, microbubble-assisted focused ultrasound (FUS). In an embodiment, transfection is carried out by electroporation or by lipid-mediated transfection or by a combination of methods for each RNA molecules.

In an embodiment, the mononuclear cell and the produced multifunctional cell can be a cytotoxic cell, such as a cytotoxic T cell or a NK cell.

In an embodiment, the method of production can further comprise (i) disrupting the expression of the endogenous T cell receptor (TCR) such that the TCR is expressed on the cell surface at reduced levels and/or (ii) disrupting the expression of the HLA/MHC complex such that the complex is expressed on the cell surface at reduced levels. In an embodiment, the endogenous TCR and/or the HLA/MHC complex is not detectably expressed on the surface of the multifunctional cell.

In another aspect, the present disclosure provides a multifunctional cell, preferably a cytotoxic T cell, which is produced by the method for producing multifunctional cells disclosed herein. Such produced cells can be used in a method of treating a disease or disorder in a patient characterized by expression of an antigen to which the immune receptor binds, said method comprising administering to the patient the multifunctional cell. In an embodiment, the disease or disorder is cancer, preferably wherein the cancer is glioblastoma.

In an aspect, the present disclosure provides a method of treating a disease or disorder in a patient comprising administering to the patient a multifunctional cell transiently expressing an immune receptor and two or more cytokines, wherein the disease or disorder is characterized by the expression of an antigen to which the immune receptor binds, wherein the cell comprises one or more exogenous RNA molecules encoding the immune receptor and the two or more cytokines, preferably wherein the patient is a human. The present disclosure also provides a multifunctional cell transiently expressing an immune receptor and two or more cytokines for use in manufacturing a medicament for treating a disease or disorder in a patient, wherein the disease or disorder is characterized by the expression of an antigen to which the immune receptor binds.

In an aspect, the present disclosure provides one or more exogenous RNA molecules encoding an immune receptor and two or more cytokines for use in a method of treating a disease or disorder in a patient, said method comprising administering to the patient the one or more exogenous RNA molecules, wherein the disease or disorder is characterized by the expression of an antigen to which the immune receptor binds, preferably wherein the patient is a human. The present disclosure also provides one or more exogenous RNA molecules encoding an immune receptor and two or more cytokines for use in manufacturing a medicament for treating a disease or disorder in a patient, wherein the disease or disorder is characterized by the expression of an antigen to which the immune receptor binds.

In an aspect, the present disclosure provides a method of treating cancer in a patient comprising administering to the patient one or more exogenous RNA molecules encoding an immune receptor and two or more cytokines, wherein the cancer is characterized by the expression of a cancer antigen to which the immune receptor binds, preferably wherein the patient is a human.

In an aspect the present disclosure provides a multifunctional cell transiently expressing an immune receptor and one or more cytokines, wherein the cell comprises one or more exogenous RNA molecules encoding the immune receptor and the one or more cytokines, wherein the one or more cytokines preferably is IL-12 and the cell does not contain any exogenous DNA molecules encoding IL-12, optionally also not containing any exogenous DNA encoding the immune receptor. The present disclosure also encompasses the use of such a multifunctional cell in the treatment of diseases and disorders as described herein and methods for the production of such a multifunctional cell.

Although possibly not specified with each aspect of the disclosure, the various embodiments set out in the disclosure can be applied to the other various aspects of the present disclosure involving the use or production of the multifunctional cells comprising one or more RNA molecules encoding an immune receptor and the cytokine(s) disclosed herein, as well as the use or production of the one or more RNA molecules.

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. Certain of these elements may be listed with specific embodiments; however, it should be understood that these elements may be combined in any manner and in any number to create additional embodiments, and which apply to the various aspects. The variously described examples and preferred embodiments should not be construed to limit the present disclosure to only the explicitly described embodiments. This description should be understood to disclose and encompass embodiments which combine the explicitly described embodiments with any number of the disclosed and/or preferred elements. Furthermore, any permutations and combinations of all described elements in this application should be considered disclosed by this description unless the context indicates otherwise.

In particular embodiments, the embodiments and aspects pertaining to multifunctional cells according to the present disclosure can be combined with the methods of the present disclosure, in particular methods of treatment or methods of production.

The term “about” means approximately or nearly, and in the context of a numerical value or range set forth herein preferably means+/−10% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the disclosure otherwise claimed. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

Indications of relative amounts of a component characterized by a generic term are meant to refer to the total amount of all specific variants or members covered by said generic term. If a certain component defined by a generic term is specified to be present in a certain relative amount, and if this component is further characterized to be a specific variant or member covered by the generic term, it is meant that no other variants or members covered by the generic term are additionally present such that the total relative amount of components covered by the generic term exceeds the specified relative amount; more preferably no other variants or members covered by the generic term are present at all.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

Terms such as “reduce” or “inhibit” as used herein means the ability to cause an overall decrease, preferably of 5% or greater, 10% or greater, 20% or greater, more preferably of 50% or greater, and most preferably 75% or greater, in the level. The term “inhibit” or similar phrases includes a complete or essentially complete inhibition, i.e., a reduction to zero or essentially to zero.

Terms such as “increase” or “enhance” preferably relate to an increase or enhancement by about at least 10%, preferably at least 20%, preferably at least 30%, more preferably at least 40%, more preferably at least 50%, even more preferably at least 80%, and most preferably at least 100%.

“Physiological pH” as used herein refers to a pH of about 7.4. In some embodiments, physiological pH is from 7.3 to 7.5. In some embodiments, physiological pH is from 7.35 to 7.45. In some embodiments, physiological pH is 7.3, 7.35, 7.4, 7.45, or 7.5.

As used in the present disclosure, “% w/v” refers to weight by volume percent, which is a unit of concentration measuring the amount of solute in grams (g) expressed as a percent of the total volume of solution in milliliters (mL).

As used in the present disclosure, “% by weight” refers to weight percent, which is a unit of concentration measuring the amount of a substance in grams (g) expressed as a percent of the total weight of the total composition in grams (g).

As used in the present disclosure, “mol %” is defined as the ratio of the number of moles of one component to the total number of moles of all components, multiplied by 100.

As used in the present disclosure, “mol % of the total lipid” is defined as the ratio of the number of moles of one lipid component to the total number of moles of all lipids, multiplied by 100. In this context, in some embodiments, the term “total lipid” includes lipids and lipid-like material.

As used herein, the terms “room temperature” and “ambient temperature” are used interchangeably herein and refer to temperatures from at least about 15° C., e.g., from about 15° C. to about 35° C., from about 15° C. to about 30° C., from about 15° C. to about 25° C., or from about 17° C. to about 22° C. Such temperatures will include 15° C., 16° C., 17° C., 18° C., 19° C., 20° C., 21° C. and 22° C.

In the present specification, a structural formula of a compound may represent a certain isomer of said compound. It is to be understood, however, that the present disclosure includes all isomers such as geometrical isomers, optical isomers based on an asymmetrical carbon, stereoisomers, tautomers and the like which occur structurally and isomer mixtures and is not limited to the description of the formula. “Isomers” are compounds having the same molecular formula but differ in structure (“structural isomers”) or in the geometrical (spatial) positioning of the functional groups and/or atoms (“stereoisomers”). “Enantiomers” are a pair of stereoisomers which are non-superimposable mirror-images of each other. A “racemic mixture” or “racemate” contains a pair of enantiomers in equal amounts and is denoted by the prefix (±). “Diastereomers” are stereoisomers which are non-superimposable and which are not mirror-images of each other. “Tautomers” are structural isomers of the same chemical substance that spontaneously and reversibly interconvert into each other, even when pure, due to the migration of individual atoms or groups of atoms; i.e., the tautomers are in a dynamic chemical equilibrium with each other. An example of tautomers are the isomers of the keto-enol-tautomerism. “Conformers” are stereoisomers that can be interconverted just by rotations about formally single bonds, and include—in particular—those leading to different 3-dimentional forms of (hetero) cyclic rings, such as chair, half-chair, boat, and twist-boat forms of cyclohexane.

The term “net charge” refers to the charge on a whole object, such as a compound or particle.

An ion having an overall net positive charge is a cation, while an ion having an overall net negative charge is an anion. Thus, according to the disclosure, an anion is an ion with more electrons than protons, giving it a net negative charge; and a cation is an ion with fewer electrons than protons, giving it a net positive charge.

Terms as “charged”, “net charge”, “negatively charged”, “positively charged” “neutral”, with reference to a given compound or particle, refer to the electric net charge of the given compound or particle when dissolved or suspended in water at pH 7.0.

The term “nucleic acid” according to the disclosure also comprises a chemical derivatization of a nucleic acid on a nucleotide base, on the sugar or on the phosphate, and nucleic acids containing non-natural nucleotides and nucleotide analogs. In some embodiments, the nucleic acid is a deoxyribonucleic acid (DNA) or a ribonucleic acid (RNA). In general, a nucleic acid molecule or a nucleic acid sequence refers to a nucleic acid which is preferably deoxyribonucleic acid (DNA) or ribonucleic acid (RNA). According to the disclosure, nucleic acids comprise genomic DNA, cDNA, mRNA, viral RNA, recombinantly prepared and chemically synthesized molecules. According to the disclosure, a nucleic acid may be in the form of a single-stranded or double-stranded and linear or covalently closed circular molecule.

According to the disclosure “nucleic acid sequence” refers to the sequence of nucleotides in a nucleic acid, e.g.; a ribonucleic acid (RNA) or a deoxyribonucleic acid (DNA). The term may refer to an entire nucleic acid molecule (such as to the single strand of an entire nucleic acid molecule) or to a part (e.g., a fragment) thereof.

According to the present disclosure, the term “RNA” or “RNA molecule” relates to a molecule which comprises ribonucleotide residues and which is preferably entirely or substantially composed of ribonucleotide residues. The term “ribonucleotide” relates to a nucleotide with a hydroxyl group at the 2′-position of a β-D-ribofuranosyl group. The term “RNA” comprises double-stranded RNA, single stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of an RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs, particularly analogs of naturally occurring RNAs.

According to the disclosure, RNA may be single-stranded or double-stranded. In some embodiments of the present disclosure, single-stranded RNA is preferred. The term “single-stranded RNA” generally refers to an RNA molecule to which no complementary nucleic acid molecule (typically no complementary RNA molecule) is associated. Single-stranded RNA may contain self-complementary sequences that allow parts of the RNA to fold back and to form secondary structure motifs including without limitation base pairs, stems, stem loops and bulges. Single-stranded RNA can exist as minus strand [(−) strand] or as plus strand [(+) strand]. The (+) strand is the strand that comprises or encodes genetic information. The genetic information may be for example a polynucleotide sequence encoding a protein. When the (+) strand RNA encodes a protein, the (+) strand may serve directly as template for translation (protein synthesis). The (−) strand is the complement of the (+) strand. In the case of double-stranded RNA, (+) strand and (−) strand are two separate RNA molecules, and both these RNA molecules associate with each other to form a double-stranded RNA (“duplex RNA”).

According to the disclosure an “exogenous RNA/DNA” is an RNA or DNA molecule, which does not occur naturally within a cell, but which has been artificially introduced into the cell, for example by transfection.

The term “stability” of RNA relates to the “half-life” of RNA. “Half-life” relates to the period of time which is needed to eliminate half of the activity, amount, or number of molecules. In the context of the present disclosure, the half-life of an RNA is indicative for the stability of said RNA. The half-life of RNA may influence the “duration of expression” of the RNA. It can be expected that RNA having a long half-life will be expressed for an extended time period.

The term “translation efficiency” relates to the amount of translation product provided by an RNA molecule within a particular period of time.

“Fragment”, with reference to a nucleic acid sequence, relates to a part of a nucleic acid sequence, i.e.; a sequence which represents the nucleic acid sequence shortened at the 5′- and/or 3′-end(s). Preferably, a fragment of a nucleic acid sequence comprises at least 80%, preferably at least 90%, 95%, 96%, 97%, 98%, or 99% of the nucleotide residues from said nucleic acid sequence. In the present disclosure those fragments of RNA molecules are preferred which retain RNA stability and/or translational efficiency.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises e.g. at least 1%, at least 2%, at least 3%, at least 4%, at least 5%, at least 10%, at least 20%, at least 30%, at least 40%, at least 50%, at least 60%, at least 70%, at least 80%, at least 90% of the amino acid residues from an amino acid sequence.

The term “variant” with respect to, for example, nucleic acid and amino acid sequences, according to the disclosure includes any variants, in particular mutants, viral strain variants, splice variants, conformations, isoforms, allelic variants, species variants and species homologs, in particular those which are naturally present. An allelic variant relates to an alteration in the normal sequence of a gene, the significance of which is often unclear. Complete gene sequencing often identifies numerous allelic variants for a given gene. With respect to nucleic acid molecules, the term “variant” includes degenerate nucleic acid sequences, wherein a degenerate nucleic acid according to the disclosure is a nucleic acid that differs from a reference nucleic acid in codon sequence due to the degeneracy of the genetic code. A species homolog is a nucleic acid or amino acid sequence with a different species of origin from that of a given nucleic acid or amino acid sequence. A virus homolog is a nucleic acid or amino acid sequence with a different virus of origin from that of a given nucleic acid or amino acid sequence.

In some embodiments, a fragment or variant of an amino acid sequence (peptide or protein) is a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In some embodiments, the modifications in the amino acid sequence of the parent molecule or sequence do not significantly affect or alter the characteristics of the molecule or sequence. In different embodiments, the function of the functional fragment or functional variant may be reduced but still significantly present, e.g., function of the functional fragment or functional variant may be at least 50%, at least 60%, at least 70%, at least 80%, or at least 90% of the parent molecule or sequence. However, in other embodiments, function of the functional 5 fragment or functional variant may be enhanced compared to the parent molecule or sequence.

Nucleic acid variants include single or multiple nucleotide deletions, additions, mutations, substitutions and/or insertions in comparison with the reference nucleic acid. Deletions include removal of one or more nucleotides from the reference nucleic acid. Addition variants comprise 5′- and/or 3′-terminal fusions of one or more nucleotides, such as 1, 2, 3, 5, 10, 20, 30, 50, or more nucleotides. In the case of substitutions, at least one nucleotide in the sequence is removed and at least one other nucleotide is inserted in its place (such as transversions and transitions). Mutations include abasic sites, crosslinked sites, and chemically altered or modified bases. Insertions include the addition of at least one nucleotide into the reference nucleic acid.

According to the disclosure, “nucleotide change” can refer to single or multiple nucleotide deletions, additions, mutations, substitutions and/or insertions in comparison with the reference nucleic acid. In some embodiments, a “nucleotide change” is selected from the group consisting of a deletion of a single nucleotide, the addition of a single nucleotide, the mutation of a single nucleotide, the substitution of a single nucleotide and/or the insertion of a single nucleotide, in comparison with the reference nucleic acid. According to the disclosure, a nucleic acid variant can comprise one or more nucleotide changes in comparison with the reference nucleic acid.

Variants of specific nucleic acid sequences preferably have at least one functional property of said specific sequences and preferably are functionally equivalent to said specific sequences, e.g., nucleic acid sequences exhibiting properties identical or similar to those of the specific nucleic acid sequences.

As described below, some embodiments of the present disclosure are characterized, inter alia, by nucleic acid sequences that are homologous to other nucleic acid sequences. These homologous sequences are variants of other nucleic acid sequences.

Preferably the degree of identity between a given nucleic acid sequence and a nucleic acid sequence which is a variant of said given nucleic acid sequence will be at least 70%, preferably at least 75%, preferably at least 80%, more preferably at least 85%, even more preferably at least 90% or most preferably at least 95%, 96%, 97%, 98% or 99%. The degree of identity is preferably given for a region of at least about 30, at least about 50, at least about 70, at least about 90, at least about 100, at least about 150, at least about 200, at least about 250, at least about 300, or at least about 400 nucleotides. In preferred embodiments, the degree of identity is given for the entire length of the reference nucleic acid sequence.

“Sequence similarity” indicates the percentage of amino acids that either are identical or that represent conservative amino acid substitutions. “Sequence identity” between two polypeptide or nucleic acid sequences indicates the percentage of amino acids or nucleotides that are identical between the sequences.

The term “% identical” is intended to refer, in particular, to a percentage of nucleotides which are identical in an optimal alignment between two sequences to be compared, with said percentage being purely statistical, and the differences between the two sequences may be randomly distributed over the entire length of the sequence and the sequence to be compared may comprise additions or deletions in comparison with the reference sequence, in order to obtain optimal alignment between two sequences. Comparisons of two sequences are usually carried out by comparing said sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2:482, with the aid of the local homology algorithm by Needleman and Wunsch, 1970, J. Mol. Biol. 48:443, and with the aid of the similarity search algorithm by Pearson and Lipman, 1988, Proc. Natl Acad. Sci. USA 85:2444 or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.).

Percentage identity is obtained by determining the number of identical positions in which the sequences to be compared correspond, dividing this number by the number of positions compared and multiplying this result by 100.

For example, the BLAST program “BLAST 2 sequences” which is available on the website http://www.ncbi.nlm.nih.gov/blast/bl2seq/wblast2.cgi may be used.

A nucleic acid is “capable of hybridizing” or “hybridizes” to another nucleic acid if the two sequences are complementary with one another. A nucleic acid is “complementary” to another nucleic acid if the two sequences are capable of forming a stable duplex with one another. According to the disclosure, hybridization is preferably carried out under conditions which allow specific hybridization between polynucleotides (stringent conditions). Stringent conditions are described, for example, in Molecular Cloning: A Laboratory Manual, J. Sambrook et al., Editors, 2nd Edition, Cold Spring Harbor Laboratory press, Cold Spring Harbor, New York, 1989 or Current Protocols in Molecular Biology, F. M. Ausubel et al., Editors, John Wiley & Sons, Inc., New York and refer, for example, to hybridization at 65° C. in hybridization buffer (3.5×SSC, 0.02% Ficoll, 0.02% polyvinylpyrrolidone, 0.02% bovine serum albumin, 2.5 mM NaH2PO4 (pH 7), 0.5% SDS, 2 mM EDTA). SSC is 0.15 M sodium chloride/0.15 M sodium citrate, pH 7. After hybridization, the membrane to which the DNA has been transferred is washed, for example, in 2×SSC at room temperature and then in 0.1-0.5×SSC/0.1×SDS at temperatures of up to 68° C.

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or “fully complementary” means that all the contiguous residues of a nucleic acid sequence will hydrogen bond with the same number of contiguous residues in a second nucleic acid sequence. Preferably, the degree of complementarity according to the disclosure is at least 70%, preferably at least 75%, preferably at least 80%, more preferably at least 85%, even more preferably at least 90% or most preferably at least 95%, 96%, 97%, 98% or 99%. Most preferably, the degree of complementarity according to the disclosure is 100%.

The term “derivative” comprises any chemical derivatization of a nucleic acid on a nucleotide base, on the sugar or on the phosphate. The term “derivative” also comprises nucleic acids which contain nucleotides and nucleotide analogs not occurring naturally. Preferably, a derivatization of a nucleic acid increases its stability.

A “nucleic acid sequence which is derived from a nucleic acid sequence” refers to a nucleic acid which is a variant of the nucleic acid from which it is derived. Preferably, a sequence which is a variant with respect to a specific sequence, when it replaces the specific sequence in an RNA molecule retains RNA stability and/or translational efficiency.

“nt” is an abbreviation for nucleotide; or for nucleotides, preferably consecutive nucleotides in a nucleic acid molecule.

According to the disclosure, the term “codon” refers to a base triplet in a coding nucleic acid that specifies which amino acid will be added next during protein synthesis at the ribosome.

The terms “transcription” and “transcribing” relate to a process during which a nucleic acid molecule with a particular nucleic acid sequence (the “nucleic acid template”) is read by an RNA polymerase so that the RNA polymerase produces a single-stranded RNA molecule. During transcription, the genetic information in a nucleic acid template is transcribed. The nucleic acid template may be DNA; however, e.g.; in the case of transcription from an alphaviral nucleic acid template, the template is typically RNA. Subsequently, the transcribed RNA may be translated into protein. According to the present disclosure, the term “transcription” comprises “in vitro transcription”, wherein the term “in vitro transcription” relates to a process wherein RNA, in particular mRNA, is in vitro synthesized in a cell-free system. Preferably, cloning vectors are applied for the generation of transcripts. These cloning vectors are generally designated as transcription vectors and are according to the present disclosure encompassed by the term “vector”. The cloning vectors are preferably plasmids. According to the present disclosure, RNA preferably is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

The single-stranded nucleic acid molecule produced during transcription typically has a nucleic acid sequence that is the complementary sequence of the template.

According to the disclosure, the terms “template” or “nucleic acid template” or “template nucleic acid” generally refer to a nucleic acid sequence that may be replicated or transcribed.

“Nucleic acid sequence transcribed from a nucleic acid sequence” and similar terms refer to a nucleic acid sequence, where appropriate as part of a complete RNA molecule, which is a transcription product of a template nucleic acid sequence. Typically, the transcribed nucleic acid sequence is a single-stranded RNA molecule.

“3′ end of a nucleic acid” refers according to the disclosure to that end which has a free hydroxy group. In a diagrammatic representation of double-stranded nucleic acids, in particular DNA, the 3′ end is always on the right-hand side. “5′ end of a nucleic acid” refers according to the disclosure to that end which has a free phosphate group. In a diagrammatic representation of double-strand nucleic acids, in particular DNA, the 5′ end is always on the left-hand side.

| | | |
|---|---|---|
| 5′ end | 5′--P-NNNNNNN-OH-3′ | 3′ end |
| | 3′-HO-NNNNNNN-P--5′ | |

“Upstream” describes the relative positioning of a first element of a nucleic acid molecule with respect to a second element of that nucleic acid molecule, wherein both elements are comprised in the same nucleic acid molecule, and wherein the first element is located nearer to the 5′ end of the nucleic acid molecule than the second element of that nucleic acid molecule. The second element is then said to be “downstream” of the first element of that nucleic acid molecule. An element that is located “upstream” of a second element can be synonymously referred to as being located “5” of that second element. For a double-stranded nucleic acid molecule, indications like “upstream” and “downstream” are given with respect to the (+) strand.

According to the disclosure, “functional linkage” or “functionally linked” relates to a connection within a functional relationship. A nucleic acid is “functionally linked” if it is functionally related to another nucleic acid sequence. For example, a promoter is functionally linked to a coding sequence if it influences transcription of said coding sequence. Functionally linked nucleic acids are typically adjacent to one another, where appropriate separated by further nucleic acid sequences, and, in particular embodiments, are transcribed by RNA polymerase to give a single RNA molecule (common transcript).

In particular embodiments, a nucleic acid is functionally linked according to the disclosure to expression control sequences which may be homologous or heterologous with respect to the nucleic acid.

The term “expression control sequence” comprises according to the disclosure promoters, ribosome-binding sequences and other control elements which control transcription of a gene or translation of the derived RNA. In particular embodiments of the disclosure, the expression control sequences can be regulated. The precise structure of expression control sequences may vary depending on the species or cell type but usually includes 5′-untranscribed and 5′- and 3′-untranslated sequences involved in initiating transcription and translation, respectively. More specifically, 5′-untranscribed expression control sequences include a promoter region which encompasses a promoter sequence for transcription control of the functionally linked gene. Expression control sequences may also include enhancer sequences or upstream activator sequences. An expression control sequence of a DNA molecule usually includes 5′-untranscribed and 5′- and 3′-untranslated sequences such as TATA box, capping sequence, CAAT sequence and the like.

The nucleic acid sequences specified herein, in particular transcribable and coding nucleic acid sequences, may be combined with any expression control sequences, in particular promoters, which may be homologous or heterologous to said nucleic acid sequences, with the term “homologous” referring to the fact that a nucleic acid sequence is also functionally linked naturally to the expression control sequence, and the term “heterologous” referring to the fact that a nucleic acid sequence is not naturally functionally linked to the expression control sequence.

A transcribable nucleic acid sequence, in particular a nucleic acid sequence coding for a peptide or protein, and an expression control sequence are “functionally” linked to one another, if they are covalently linked to one another in such a way that transcription or expression of the transcribable and in particular coding nucleic acid sequence is under the control or under the influence of the expression control sequence. If the nucleic acid sequence is to be translated into a functional peptide or protein, induction of an expression control sequence functionally linked to the coding sequence results in transcription of said coding sequence, without causing a frame shift in the coding sequence or the coding sequence being unable to be translated into the desired peptide or protein.

The term “promoter” or “promoter region” refers to a nucleic acid sequence which controls synthesis of a transcript, e.g. a transcript comprising a coding sequence, by providing a recognition and binding site for RNA polymerase. The promoter region may include further recognition or binding sites for further factors involved in regulating transcription of said gene. A promoter may control transcription of a prokaryotic or eukaryotic gene. A promoter may be “inducible” and initiate transcription in response to an inducer, or may be “constitutive” if transcription is not controlled by an inducer. An inducible promoter is expressed only to a very small extent or not at all, if an inducer is absent. In the presence of the inducer, the gene is “switched on” or the level of transcription is increased. This is usually mediated by binding of a specific transcription factor.

The term “core promoter” refers to a nucleic acid sequence that is comprised by the promoter. The core promoter is typically the minimal portion of the promoter required to properly initiate transcription. The core promoter typically includes the transcription start site and a binding site for RNA polymerase.

A “polymerase” generally refers to a molecular entity capable of catalyzing the synthesis of a polymeric molecule from monomeric building blocks. An “RNA polymerase” is a molecular entity capable of catalyzing the synthesis of an RNA molecule from ribonucleotide building blocks. A “DNA polymerase” is a molecular entity capable of catalyzing the synthesis of a DNA molecule from deoxy ribonucleotide building blocks. For the case of DNA polymerases and RNA polymerases, the molecular entity is typically a protein or an assembly or complex of multiple proteins. Typically, a DNA polymerase synthesizes a DNA molecule based on a template nucleic acid, which is typically a DNA molecule. Typically, an RNA polymerase synthesizes an RNA molecule based on a template nucleic acid, which is either a DNA molecule (in that case the RNA polymerase is a DNA-dependent RNA polymerase, DdRP), or is an RNA molecule (in that case the RNA polymerase is an RNA-dependent RNA polymerase, RdRP).

An “RNA-dependent RNA polymerase” or “RdRP”, is an enzyme that catalyzes the transcription of RNA from an RNA template. In the case of alphaviral RNA-dependent RNA polymerase, sequential synthesis of (−) strand complement of genomic RNA and of (+) strand genomic RNA leads to RNA replication. RNA-dependent RNA polymerase is thus synonymously referred to as “RNA replicase” or simply “replicase”. In nature, RNA-dependent RNA polymerases are typically encoded by all RNA viruses except retroviruses. Typical representatives of viruses encoding an RNA-dependent RNA polymerase are alphaviruses.

According to the present disclosure, “RNA replication” generally refers to an RNA molecule synthesized based on the nucleotide sequence of a given RNA molecule (template RNA molecule). The RNA molecule that is synthesized may be, e.g., identical or complementary to the template RNA molecule. In general, RNA replication may occur via synthesis of a DNA intermediate, or may occur directly by RNA-dependent RNA replication mediated by an RNA-dependent RNA polymerase (RdRP). In the case of alphaviruses, RNA replication does not occur via a DNA intermediate, but is mediated by a RNA-dependent RNA polymerase (RdRP): a template RNA strand (first RNA strand)—or a part thereof—serves as template for the synthesis of a second RNA strand that is complementary to the first RNA strand or to a part thereof. The second RNA strand—or a part thereof—may in turn optionally serve as a template for synthesis of a third RNA strand that is complementary to the second RNA strand or to a part thereof. Thereby, the third RNA strand is identical to the first RNA strand or to a part thereof. Thus, RNA-dependent RNA polymerase is capable of directly synthesizing a complementary RNA strand of a template, and of indirectly synthesizing an identical RNA strand (via a complementary intermediate strand).

According to the disclosure, the term “gene” refers to a particular nucleic acid sequence which is responsible for producing one or more cellular products and/or for achieving one or more intercellular or intracellular functions. More specifically, said term relates to a nucleic acid section (typically DNA; but RNA in the case of RNA viruses) which comprises a nucleic acid coding for a specific protein or a functional or structural RNA molecule.

An “isolated molecule” as used herein, is intended to refer to a molecule which is substantially free of other molecules such as other cellular material. The term “isolated nucleic acid” means according to the disclosure that the nucleic acid has been (i) amplified in vitro, for example by polymerase chain reaction (PCR), (ii) recombinantly produced by cloning, (iii) purified, for example by cleavage and gel-electrophoretic fractionation, or (iv) synthesized, for example by chemical synthesis. An isolated nucleic acid is a nucleic acid available to manipulation by recombinant techniques.

The term “vector” is used here in its most general meaning and comprises any intermediate vehicles for a nucleic acid which, for example, enable said nucleic acid to be introduced into prokaryotic and/or eukaryotic host cells and, where appropriate, to be integrated into a genome. Such vectors are preferably replicated and/or expressed in the cell. Vectors comprise plasmids, phagemids, virus genomes, and fractions thereof.

The term “recombinant” in the context of the present disclosure means “made through genetic engineering”. Preferably, a “recombinant object” such as a recombinant cell in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

According to the disclosure, the term “expression” is used in its most general meaning and comprises production of RNA and/or protein. It also comprises partial expression of nucleic acids. Furthermore, expression may be transient or stable. With respect to RNA, the term “expression” or “translation” relates to the process in the ribosomes of a cell by which a strand of coding RNA (e.g. messenger RNA) directs the assembly of a sequence of amino acids to make a peptide or protein.

According to the disclosure, the term “mRNA” means “messenger-RNA” and relates to a transcript which is typically generated by using a DNA template and encodes a peptide or protein. Typically, mRNA comprises a 5′-UTR, a protein coding region, a 3′-UTR, and a poly(A) sequence. mRNA may be generated by in vitro transcription from a DNA template. The in vitro transcription methodology is known to the skilled person. For example, there is a variety of in vitro transcription kits commercially available. According to the disclosure, mRNA may be modified by stabilizing modifications and capping.

According to the disclosure, the term “primary structure”, with reference to a nucleic acid molecule, refers to the linear sequence of nucleotide monomers.

According to the disclosure, the term “secondary structure”, with reference to a nucleic acid molecule, refers to a two-dimensional representation of a nucleic acid molecule that reflects base pairings; e.g.; in the case of a single-stranded RNA molecule particularly intramolecular base pairings. Although each RNA molecule has only a single polynucleotide chain, the molecule is typically characterized by regions of (intramolecular) base pairs. According to the disclosure, the term “secondary structure” comprises structural motifs including without limitation base pairs, stems, stem loops, bulges, loops such as interior loops and multi-branch loops. The secondary structure of a nucleic acid molecule can be represented by a two-dimensional drawing (planar graph), showing base pairings (for further details on secondary structure of RNA molecules, see Auber et al., 2006; J. Graph Algorithms Appl. 10:329-351). As described herein, the secondary structure of certain RNA molecules is relevant in the context of the present disclosure.

According to the disclosure, secondary structure of a nucleic acid molecule, particularly of a single-stranded RNA molecule, is determined by prediction using the web server for RNA secondary structure prediction (http://rna.urmc.rochester.edu/RNAstructureWeb/Servers/Predict1/Predict1.html).

Preferably, according to the disclosure, “secondary structure”, with reference to a nucleic acid molecule, specifically refers to the secondary structure determined by said prediction. The prediction may also be performed or confirmed using MFOLD structure prediction (http://unafold.rna.albany.edu/?q=mfold).

According to the disclosure, a “base pair” is a structural motif of a secondary structure wherein two nucleotide bases associate with each other through hydrogen bonds between donor and acceptor sites on the bases. The complementary bases, A:U and G:C, form stable base pairs through hydrogen bonds between donor and acceptor sites on the bases; the A:U and G:C base pairs are called Watson-Crick base pairs. A weaker base pair (called Wobble base pair) is formed by the bases G and U (G:U). The base pairs A:U and G:C are called canonical base pairs. Other base pairs like G:U (which occurs fairly often in RNA) and other rare base-pairs (e.g. A:C; U:U) are called non-canonical base pairs.

According to the disclosure, “nucleotide pairing” refers to two nucleotides that associate with each other so that their bases form a base pair (canonical or non-canonical base pair, preferably canonical base pair, most preferably Watson-Crick base pair).

According to the disclosure, the terms “stem loop” or “hairpin” or “hairpin loop”, with reference to a nucleic acid molecule, all interchangeably refer to a particular secondary structure of a nucleic acid molecule, typically a single-stranded nucleic acid molecule, such as single-stranded RNA. The particular secondary structure represented by the stem loop consists of a consecutive nucleic acid sequence comprising a stem and a (terminal) loop, also called hairpin loop, wherein the stem is formed by two neighbored entirely or partially complementary sequence elements; which are separated by a short sequence (e.g. 3-10 nucleotides), which forms the loop of the stem-loop structure. The two neighbored entirely or partially complementary sequences may be defined as, e.g., stem loop elements stem 1 and stem 2. The stem loop is formed when these two neighbored entirely or partially reverse complementary sequences, e.g. stem loop elements stem 1 and stem 2, form base-pairs with each other, leading to a double stranded nucleic acid sequence comprising an unpaired loop at its terminal ending formed by the short sequence located between stem loop elements stem 1 and stem 2. Thus, a stem loop comprises two stems (stem 1 and stem 2), which—at the level of secondary structure of the nucleic acid molecule-form base pairs with each other, and which—at the level of the primary structure of the nucleic acid molecule—are separated by a short sequence that is not part of stem 1 or stem 2. For illustration, a two-dimensional representation of the stem loop resembles a lollipop-shaped structure. The formation of a stem-loop structure requires the presence of a sequence that can fold back on itself to form a paired double strand; the paired double strand is formed by stem 1 and stem 2. The stability of paired stem loop elements is typically determined by the length, the number of nucleotides of stem 1 that are capable of forming base pairs (preferably canonical base pairs, more preferably Watson-Crick base pairs) with nucleotides of stem 2, versus the number of nucleotides of stem 1 that are not capable of forming such base pairs with nucleotides of stem 2 (mismatches or bulges). According to the present disclosure, the optimal loop length is 3-10 nucleotides, more preferably 4 to 7, nucleotides, such as 4 nucleotides, 5 nucleotides, 6 nucleotides or 7 nucleotides. If a given nucleic acid sequence is characterized by a stem loop, the respective complementary nucleic acid sequence is typically also characterized by a stem loop. A stem loop is typically formed by single-stranded RNA molecules.

According to the disclosure, “disruption” or “disrupt”, with reference to a specific secondary structure of a nucleic acid molecule (e.g., a stem loop) means that the specific secondary structure is absent or altered. Typically, a secondary structure may be disrupted as a consequence of a change of at least one nucleotide that is part of the secondary structure. For example, a stem loop may be disrupted by change of one or more nucleotides that form the stem, so that nucleotide pairing is not possible.

According to the disclosure, “compensates for secondary structure disruption” or “compensating for secondary structure disruption” refers to one or more nucleotide changes in a nucleic acid sequence; more typically it refers to one or more second nucleotide changes in a nucleic acid sequence, which nucleic acid sequence also comprises one or more first nucleotide changes, characterized as follows: while the one or more first nucleotide changes, in the absence of the one or more second nucleotide changes, cause a disruption of the secondary structure of the nucleic acid sequence, the co-occurrence of the one or more first nucleotide changes and the one or more second nucleotide changes does not cause the secondary structure of the nucleic acid to be disrupted. Co-occurrence means presence of both the one or more first nucleotide changes and of the one or more second nucleotide changes. Typically, the one or more first nucleotide changes and the one or more second nucleotide changes are present together in the same nucleic acid molecule. In a specific embodiment, one or more nucleotide changes that compensate for secondary structure disruption is/are one or more nucleotide changes that compensate for one or more nucleotide pairing disruptions. Thus, in one embodiment, “compensating for secondary structure disruption” means “compensating for nucleotide pairing disruptions”, i.e. one or more nucleotide pairing disruptions, for example one or more nucleotide pairing disruptions within one or more stem loops. The one or more one or more nucleotide pairing disruptions may have been introduced by the removal of at least one initiation codon. Each of the one or more nucleotide changes that compensates for secondary structure disruption is a nucleotide change, which can each be independently selected from a deletion, an addition, a substitution and/or an insertion of one or more nucleotides. In an illustrative example, when the nucleotide pairing A:U has been disrupted by substitution of A to C (C and U are not typically suitable to form a nucleotide pair); then a nucleotide change that compensates for nucleotide pairing disruption may be substitution of U by G, thereby enabling formation of the C:G nucleotide pairing. The substitution of U by G thus compensates for the nucleotide pairing disruption. In an alternative example, when the nucleotide pairing A:U has been disrupted by substitution of A to C; then a nucleotide change that compensates for nucleotide pairing disruption may be substitution of C by A, thereby restoring formation of the original A:U nucleotide pairing. In general, in the present disclosure, those nucleotide changes compensating for secondary structure disruption are preferred which do neither restore the original nucleic acid sequence nor create novel AUG triplets. In the above set of examples, the U to G substitution is preferred over the C to A substitution.

According to the disclosure, the term “tertiary structure”, with reference to a nucleic acid molecule, refers to the three-dimensional structure of a nucleic acid molecule, as defined by the atomic coordinates.

According to the disclosure, a nucleic acid such as RNA, e.g., mRNA, may encode a peptide or protein. Accordingly, a transcribable nucleic acid sequence or a transcript thereof may contain an open reading frame (ORF) encoding a peptide or protein.

According to the disclosure, the term “nucleic acid encoding a peptide or protein” means that the nucleic acid, if present in the appropriate environment, preferably within a cell, can direct the assembly of amino acids to produce the peptide or protein during the process of translation. Preferably, coding RNA according to the disclosure is able to interact with the cellular translation machinery allowing translation of the coding RNA to yield a peptide or protein.

According to the disclosure, the term “peptide” comprises oligo- and polypeptides and refers to substances which comprise two or more, preferably 3 or more, preferably 4 or more, preferably 6 or more, preferably 8 or more, preferably 10 or more, preferably 13 or more, preferably 16 or more, preferably 20 or more, and up to preferably 50, preferably 100 or preferably 150, consecutive amino acids linked to one another via peptide bonds. The term “protein” refers to large peptides, preferably peptides having at least 151 amino acids, but the terms “peptide”, “polypeptide” and “protein” are used herein usually as synonyms.

The terms “peptide” and “protein” comprise, according to the disclosure, substances which contain not only amino acid components but also non-amino acid components such as sugars and phosphate structures, and also comprise substances containing bonds such as ester, thioether or disulfide bonds.

According to the disclosure, the terms “initiation codon” and “start codon” synonymously refer to a codon (base triplet) of an RNA molecule that is potentially the first codon that is translated by a ribosome. Such codon typically encodes the amino acid methionine in eukaryotes and a modified methionine in prokaryotes. The most common initiation codon in eukaryotes and prokaryotes is AUG. Unless specifically stated herein that an initiation codon other than AUG is meant, the terms “initiation codon” and “start codon”, with reference to an RNA molecule, refer to the codon AUG. According to the disclosure, the terms “initiation codon” and “start codon” are also used to refer to a corresponding base triplet of a deoxyribonucleic acid, namely the base triplet encoding the initiation codon of an RNA. If the initiation codon of messenger RNA is AUG, the base triplet encoding the AUG is ATG. According to the disclosure, the terms “initiation codon” and “start codon” preferably refer to a functional initiation codon or start codon, i.e., to an initiation codon or start codon that is used or would be used as a codon by a ribosome to start translation. There may be AUG codons in an RNA molecule that are not used as codons by a ribosome to start translation, e.g., due to a short distance of the codons to the cap. These codons are not encompassed by the term functional initiation codon or start codon.

According to the disclosure, the terms “start codon of the open reading frame” or “initiation codon of the open reading frame” refer to the base triplet that serves as initiation codon for protein synthesis in a coding sequence, e.g., in the coding sequence of a nucleic acid molecule found in nature. In an RNA molecule, the start codon of the open reading frame is often preceded by a 5′ untranslated region (5′-UTR), although this is not strictly required.

According to the disclosure, the terms “native start codon of the open reading frame” or “native initiation codon of the open reading frame” refer to the base triplet that serves as initiation codon for protein synthesis in a native coding sequence. A native coding sequence may be, e.g., the coding sequence of a nucleic acid molecule found in nature. In some embodiments, the present disclosure provides variants of nucleic acid molecules found in nature, which are characterized in that the native start codon (which is present in the native coding sequence) has been removed (so that it is not present in the variant nucleic acid molecule).

According to the disclosure, “first AUG” means the most upstream AUG base triplet of a messenger RNA molecule, preferably the most upstream AUG base triplet of a messenger RNA molecule that is used or would be used as a codon by a ribosome to start translation. Accordingly, “first ATG” refers to the ATG base triplet of a coding DNA sequence that encodes the first AUG. In some instances, the first AUG of a mRNA molecule is the start codon of an open reading frame, i.e., the codon that is used as start codon during ribosomal protein synthesis.

According to the disclosure, the terms “comprises the removal” or “characterized by the removal” and similar terms, with reference to a certain element of a nucleic acid variant, mean that said certain element is not functional or not present in the nucleic acid variant, compared to a reference nucleic acid molecule. Without limitation, a removal can consist of deletion of all or part of the certain element, of substitution of all or part of the certain element, or of alteration of the functional or structural properties of the certain element. The removal of a functional element of a nucleic acid sequence requires that the function is not exhibited at the position of the nucleic acid variant comprising the removal. For example, an RNA variant characterized by the removal of a certain initiation codon requires that ribosomal protein synthesis is not initiated at the position of the RNA variant characterized by the removal. The removal of a structural element of a nucleic acid sequence requires that the structural element is not present at the position of the nucleic acid variant comprising the removal. For example, a RNA variant characterized by the removal of a certain AUG base triplet, i.e., of a AUG base triplet at a certain position, may be characterized, e.g., by deletion of part or all of the certain AUG base triplet (e.g., AAUG), or by substitution of one or more nucleotides (A, U, G) of the certain AUG base triplet by any one or more different nucleotides, so that the resulting nucleotide sequence of the variant does not comprise said AUG base triplet. Suitable substitutions of one nucleotide are those that convert the AUG base triplet into a GUG, CUG or UUG base triplet, or into a AAG, ACG or AGG base triplet, or into a AUA, AUC or AUU base triplet. Suitable substitutions of more nucleotides can be selected accordingly.

The term “autologous” is used to describe anything that is derived from the same subject. For example, “autologous cell” refers to a cell derived from the same subject. Introduction of autologous cells into a subject is advantageous because these cells overcome the immunological barrier which otherwise results in rejection.

The term “allogeneic” is used to describe anything that is derived from different individuals of the same species. Two or more individuals are said to be allogeneic to one another when the genes at one or more loci are not identical.

The term “syngeneic” is used to describe anything that is derived from individuals or tissues having identical genotypes, i.e., identical twins or animals of the same inbred strain, or their tissues or cells.

The term “heterologous” is used to describe something consisting of multiple different elements. As an example, the introduction of one individual's cell into a different individual constitutes a heterologous transplant. A heterologous gene is a gene derived from a source other than the subject.

The term “transfection” relates to the introduction of nucleic acids, in particular RNA, into a cell. For purposes of the present disclosure, the term “transfection” also includes the introduction of a nucleic acid into a cell or the uptake of a nucleic acid by such cell, wherein the cell may be present in a subject, e.g., a patient, or the cell may be in vitro, e.g., outside of a patient. Thus, according to the present disclosure, a cell for transfection of a nucleic acid described herein can be present in vitro or in vivo, e.g. the cell can form part of an organ, a tissue and/or the body of a patient. According to the disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection, for example. Generally, nucleic acid encoding antigen is transiently transfected into cells. RNA can be transfected into cells to transiently express its coded protein.

According to various embodiments of the present disclosure, a nucleic acid such as RNA encoding a peptide or polypeptide is taken up by or introduced, i.e. transfected or transduced, into a cell which cell may be present in vitro or in a subject, resulting in expression of said peptide or polypeptide. The cell may, e.g., express the encoded peptide or polypeptide intracellularly (e.g. in the cytoplasm and/or in the nucleus), may secrete the encoded peptide or polypeptide, and/or may express it on the surface.

The term “macrophage” refers to a subgroup of phagocytic cells produced by the differentiation of monocytes. The term “monocyte” refers to a type of leukocyte that can differentiate into macrophages or dendritic cells. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In some embodiments, the macrophages are splenic macrophages.

“Activation” or “stimulation”, as used herein, refers to the state of a cell that has been sufficiently stimulated to induce detectable cellular proliferation, such as an immune effector cell such as T cell. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “expansion” refers to a process wherein a specific entity is multiplied. In some embodiments, the term is used in the context of an immunological response in which immune effector cells are stimulated by an antigen, proliferate, and the specific immune effector cell recognizing said antigen is amplified. In some embodiments, expansion leads to differentiation of the immune effector cells.

The terms “immune response” and “immune reaction” are used herein interchangeably in their conventional meaning and refer to an integrated bodily response to an antigen and may refer to a cellular immune response, a humoral immune response, or both. According to the disclosure, the term “immune response to” or “immune response against” with respect to an agent such as an antigen, cell or tissue, relates to an immune response such as a cellular response directed against the agent. An immune response may comprise one or more reactions selected from the group consisting of developing antibodies against one or more antigens and expansion of antigen-specific T-lymphocytes, such as CD4⁺ and CD8⁺ T-lymphocytes, e.g. CD8⁺ T-lymphocytes, which may be detected in various proliferation or cytokine production tests in vitro.

The terms “inducing an immune response” and “eliciting an immune response” and similar terms in the context of the present disclosure refer to the induction of an immune response, such as the induction of a cellular immune response, a humoral immune response, or both.

The immune response may be protective/preventive/prophylactic and/or therapeutic. The immune response may be directed against any immunogen or antigen or antigen peptide, such as against a tumor-associated antigen or a pathogen-associated antigen (e.g., an antigen of a virus (such as influenza virus (A, B, or C), CMV or RSV)). “Inducing” in this context may mean that there was no immune response against a particular antigen or pathogen before induction, but it may also mean that there was a certain level of immune response against a particular antigen or pathogen before induction and after induction said immune response is enhanced. Thus, “inducing the immune response” in this context also includes “enhancing the immune response”. In some embodiments, after inducing an immune response in an individual, said individual is protected from developing a disease such as an infectious disease or a cancerous disease or the disease condition is ameliorated by inducing an immune response. In particular against a disease or disorder which is characterized by the expression of an antigen that is bound by the immune receptor of the multifunctional cells disclosed herein.

The terms “cellular immune response”, “cellular response”, “cell-mediated immunity” or similar terms are meant to include a cellular response directed to cells characterized by expression of an antigen. The cellular response relates to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill cells such as diseased cells.

The term “humoral immune response” refers to a process in living organisms wherein antibodies are produced in response to agents and organisms, which they ultimately neutralize and/or eliminate. The specificity of the antibody response is mediated by T and/or B cells through membrane-associated receptors that bind antigen of a single specificity. Following binding of an appropriate antigen and receipt of various other activating signals, B lymphocytes divide, which produces memory B cells as well as antibody secreting plasma cell clones, each producing antibodies that recognize the identical antigenic epitope as was recognized by its antigen receptor. Memory B lymphocytes remain dormant until they are subsequently activated by their specific antigen. These lymphocytes provide the cellular basis of memory and the resulting escalation in antibody response when re-exposed to a specific antigen.

The term “antibody” as used herein, refers to an immunoglobulin molecule, which is able to specifically bind to an epitope on an antigen. In particular, the term “antibody” refers to a glycoprotein comprising at least two heavy (H) chains and two light (L) chains inter-connected by disulfide bonds. The term “antibody” includes monoclonal antibodies, recombinant antibodies, human antibodies, humanized antibodies, chimeric antibodies and combinations of any of the foregoing. Each heavy chain is comprised of a heavy chain variable region (VH) and a heavy chain constant region (CH). Each light chain is comprised of a light chain variable region (VL) and a light chain constant region (CL). The variable regions and constant regions are also referred to herein as variable domains and constant domains, respectively. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The CDRs of a VH are termed HCDR1, HCDR2 and HCDR3, the CDRs of a VL are termed LCDR1, LCDR2 and LCDR3. The variable regions of the heavy and light chains contain a binding domain that interacts with an antigen. The constant regions of an antibody comprise the heavy chain constant region (CH) and the light chain constant region (CL), wherein CH can be further subdivided into constant domain CH1, a hinge region, and constant domains CH2 and CH3 (arranged from amino-terminus to carboxy-terminus in the following order: CH1, CH2, CH3). The constant regions of the antibodies may mediate the binding of the immunoglobulin to host tissues or factors, including various cells of the immune system (e.g., effector cells) and the first component (Clq) of the classical complement system. Antibodies can be intact immunoglobulins derived from natural sources or from recombinant sources and can be immunoactive portions of intact immunoglobulins. Antibodies are typically tetramers of immunoglobulin molecules. Antibodies may exist in a variety of forms including, for example, polyclonal antibodies, monoclonal antibodies, Fv, Fab and F(ab)₂, as well as single chain antibodies and humanized antibodies.

The term “immunoglobulin” relates to proteins of the immunoglobulin superfamily, such as to antigen receptors such as antibodies or the B cell receptor (BCR). The immunoglobulins are characterized by a structural domain, i.e., the immunoglobulin domain, having a characteristic immunoglobulin (Ig) fold. The term encompasses membrane bound immunoglobulins as well as soluble immunoglobulins. Membrane bound immunoglobulins are also termed surface immunoglobulins or membrane immunoglobulins, which are generally part of the BCR. Soluble immunoglobulins are generally termed antibodies. Immunoglobulins generally comprise several chains, typically two identical heavy chains and two identical light chains which are linked via disulfide bonds. These chains are primarily composed of immunoglobulin domains, such as the V_{L} (variable light chain) domain, C_{L} (constant light chain) domain, V_{H} (variable heavy chain) domain, and the C_{H} (constant heavy chain) domains C_{H}1, C_{H}2, C_{H}3, and C_{H}4. There are five types of mammalian immunoglobulin heavy chains, i.e., α, δ, ε, γ, and μ which account for the different classes of antibodies, i.e., IgA, IgD, IgE, IgG, and IgM. As opposed to the heavy chains of soluble immunoglobulins, the heavy chains of membrane or surface immunoglobulins comprise a transmembrane domain and a short cytoplasmic domain at their carboxy-terminus. In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be naturally occurring or recombinant antigens. In the context of the present disclosure, the term “tumor antigen” refers to antigens that are common to specific hyperproliferative disorders such as cancer.

The term “epitope” refers to an antigenic determinant in a molecule such as an antigen, i.e., to a part in or fragment of the molecule, that is recognized, i.e. bound, by the immune system, for example, that is recognized by an antibody or CAR. For example, epitopes are the discrete, three-dimensional sites on an antigen, which are recognized by the immune system. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents. Preferably an epitope is capable of eliciting an immune response against the antigen or a cell expressing the antigen. Preferably, the term relates to an immunogenic portion of an antigen. An epitope of a protein such as a tumor antigen preferably comprises a continuous or discontinuous portion of said protein and is preferably between 5 and 100, preferably between 5 and 50, more preferably between 8 and 30, most preferably between 10 and 25 amino acids in length, for example, the epitope may be preferably 9, 10, 11, 12, 13, 14, 15, 16, 17, 18, 19, 20, 21, 22, 23, 24, or 25 amino acids in length.

The term “anti-tumor” as used herein, refers to a biological effect which can be manifested by a decrease in tumor volume, a decrease in the number of tumor cells, a decrease in the number of metastases, a prevention of the occurrence of tumor in the first place, an increase in life expectancy, or amelioration of various physiological symptoms associated with the cancerous condition.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease (e.g., cancer, infectious diseases) but may or may not have the disease, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In some embodiments of the present disclosure, the “individual” or “subject” is a “patient”.

The term “patient” means an individual or subject for treatment, in particular a diseased individual or subject. In some embodiments, the subject is a mammal. In some embodiments, the mammal is a human.

The following provides specific and/or preferred variants of the individual features of the disclosure. The present disclosure also contemplates as particularly preferred embodiments those embodiments, which are generated by combining two or more of the specific and/or preferred variants described for two or more of the features of the present disclosure.

### Multifunctional Cell

According to the disclosure a “mononuclear cell” is a blood cell with a round nucleus, such as a peripheral blood mononuclear cell (“PBMC”). This includes lymphocytes, such as T cell, B cell and NK cell, and monocytes. Such cells also can be obtained from tumor infiltrates, bone marrow and the spleen. Such cells also can be obtained from cell lines and cell banks.

The term “multifunctional” is to be understood as referring to a mononuclear cell, which has been altered by introduction of one or more exogenous RNA molecules into the cell and is transiently expressing from these RNA molecules an immune receptor, e.g., chimeric antigen receptor, i.e., as a first function, and two or more cytokines, i.e., as a second, third or more function, and therefor has several additional altered functions, not fulfilled by a cell that has not been altered or not occurring in an unaltered cell to the same level as in the altered cell.

In some embodiments the multifunctional cell is a T cell or NK cell, preferably a T cell. In an embodiment, the T cell is a cytotoxic T cell. In a preferred embodiment, the T cell according to the disclosure or used in a method according to the disclosure exhibits a reduced expression of the T cell receptor and/or of the HLA/MHC complex. Preferably, the reduced level of expression is no more than 35%, 30%, 25%, 20%, 15%, 10%, 5%, 4%, 3%, 2%, or 1% of the wild-type level of expression. In a preferred embodiment, the TCR and/or the HLA/MHC complex is not detectably expressed on the surface of the T cell. In some embodiments, the expression of the TCR and/or the HLA/MHC complex is determined using a FACS assay.

The multifunctional cell according to the present disclosure does not contain any functional exogenous DNA molecules encoding the immune receptor and/or the two or more cytokines that can be transcribed to express the immune receptor and/or the two or more cytokines. Preferably, the multifunctional cell does not comprise any exogenous DNA encoding the immune receptor and/or the two or more cytokines.

In a preferred embodiment, the multifunctional cell used in a method according to the present disclosure does not contain DNA encoding the CAR, preferably the same CAR as encoded by the exogenous RNA molecule. In a preferred embodiment, the multifunctional cell used in a method according to the present disclosure does not contain DNA encoding the two or more cytokines, preferably the same two or more cytokines as encoded by the exogenous RNA molecule.

In a preferred embodiment, the multifunctional cell used in a method according to the present disclosure does not express an exogenous integrase, which is preferably capable of integrating DNA sequences into the genome of the cell.

### Immune Receptor

According to the disclosure, the term “CAR” (or “chimeric antigen receptor”) relates to an artificial receptor comprising a single molecule or a complex of molecules which recognizes, i.e., binds to, a target structure (e.g., an antigen) on a target cell such as a cancer cell (e.g., by binding of an antigen binding domain to an antigen expressed on the surface of the target cell) and may confer specificity onto an immune effector cell such as a T cell expressing said CAR on the cell surface. Preferably, recognition of the target structure by a CAR results in activation of an immune effector cell expressing said CAR. A CAR may comprise one or more protein units said protein units comprising one or more domains as described herein. The term “CAR” does not include T cell receptors.

Adoptive cell transfer therapy with CAR-engineered T cells expressing chimeric antigen receptors is a promising anti-cancer therapeutic as CAR-modified T cells can be engineered to target virtually any tumor antigen. For example, patient's T cells may be genetically engineered (genetically modified) to express CARs specifically directed towards antigens on the patient's tumor cells, then infused back into the patient.

In some embodiments, the CAR binds to an antigen expressed on a tumor cell, preferably a tumor cell of a glioblastoma. In a preferred embodiment, the CAR according to the disclosure is capable of binding to a NKG2D ligand.

### Cytokines

The multifunctional cells express two or more cytokines or a variant thereof. In a preferred embodiment, the cells express two cytokines. Examples of cytokines include interferons, such as interferon-alpha (IFN-α), in particular interferon-α2, or interferon-gamma (IFN-γ), interleukins, such as IL-2, IL-7, IL-10, IL-12, IL-15, IL-18 and IL-23, colony stimulating factors, such as M-CSF and GM-CSF, and tumor necrosis factor. Included with the term cytokines are subtypes of a particular cytokines, such as subtypes of IFN-α, for example, IFN-α1, IFN-α2, IFN-α8, IFN-α10, IFN-α14 or IFN-α21. Preferred cytokines are those who binding receptor is expressed by the multifunctional cells. For example, such cytokines can act in a positive feedback loop to activate the cells in which they are expressed.

The term “cytokines” relates to proteins which have a molecular weight of about 5 to 60 kDa and which participate in cell signaling (e.g., paracrine, endocrine, and/or autocrine signaling). In particular, when released, cytokines exert an effect on the behavior of cells around the place of their release. Examples of cytokines include lymphokines, interleukins, chemokines, interferons, and tumor necrosis factors (TNFs). According to the present disclosure, cytokines do not include hormones or growth factors. Cytokines differ from hormones in that (i) they usually act at much more variable concentrations than hormones and (ii) generally are made by a broad range of cells (nearly all nucleated cells can produce cytokines). Interferons are usually characterized by antiviral, antiproliferative and immunomodulatory activities. Interferons are proteins that alter and regulate the transcription of genes within a cell by binding to interferon receptors on the regulated cell's surface, thereby preventing viral replication within the cells. The interferons can be grouped into two types. Particular examples of cytokines include erythropoietin (EPO), colony stimulating factor (CSF), granulocyte colony stimulating factor (G-CSF), granulocyte-macrophage colony stimulating factor (GM-CSF), tumor necrosis factor (TNF), bone morphogenetic protein (BMP), interferon alpha (IFNα), interferon beta (IFNβ), interferon gamma (INFγ), interleukin 2 (IL-2), interleukin 4 (IL-4), interleukin 7 (IL-7), interleukin 10 (IL-10), interleukin 11 (IL-11), interleukin 12 (IL-12), interleukin 15 (IL-15), interleukin 18 (IL-18), interleukin 21 (IL-21) and interleukin 23 (IL-23), as well as variants and derivatives thereof.

According to the disclosure, a cytokine may be a naturally occurring cytokine or a functional fragment or variant thereof. A cytokine may be a human cytokine or may be derived from any vertebrate, especially any mammal. Further, a cytokine can be a molecule that activates the cytokine receptor, thus providing the same function of the cytokine to the cell on which the cytokine receptor is expressed.

Interferons (IFNs) are a group of signaling proteins made and released by host cells in response to the presence of several pathogens, such as viruses, bacteria, parasites, and also tumor cells. In a typical scenario, a virus-infected cell will release interferons causing nearby cells to heighten their anti-viral defenses.

Based on the type of receptor through which they signal, interferons are typically divided among three classes: type I interferon, type II interferon, and type III interferon.

All type I interferons bind to a specific cell surface receptor complex known as the IFN-α/β receptor (IFNAR) that consists of IFNAR1 and IFNAR2 chains. The type I interferons present in humans are IFNα, IFNβ, IFNε, IFNκ and IFNω. In general, type I interferons are produced when the body recognizes a virus that has invaded it. They are produced by fibroblasts and monocytes. Once released, type I interferons bind to specific receptors on target cells, which leads to expression of proteins that will prevent the virus from producing and replicating its RNA and DNA.

The IFNα proteins are produced mainly by plasmacytoid dendritic cells (pDCs). They are mainly involved in innate immunity against viral infection. The genes responsible for their synthesis come in 13 subtypes that are called IFNA1, IFNA2, IFNA4, IFNA5, IFNA6, IFNA7, IFNA8, IFNA10, IFNA13, IFNA14, IFNA16, IFNA17, IFNA21. These genes are found together in a cluster on chromosome 9.

The IFNβ proteins are produced in large quantities by fibroblasts. They have antiviral activity that is involved mainly in innate immune response. Two types of IFNβ have been described, IFNβ1 and IFNβ3. The natural and recombinant forms of IFNβ1 have antiviral, antibacterial, and anticancer properties.

Type II interferon (IFNγ in humans) is also known as immune interferon and is activated by IL12. Furthermore, type II interferons are released by cytotoxic T cells and T helper cells.

Type III interferons signal through a receptor complex consisting of IL-10R2 (also called CRF2-4) and IFNLR1 (also called CRF2-12). Although discovered more recently than type I and type II IFNs, recent information demonstrates the importance of type III IFNs in some types of virus or fungal infections.

In general, type I and II interferons are responsible for regulating and activating the immune response.

According to the disclosure, a type I interferon is preferably IFNα or IFNβ, more preferably IFNα, even more preferably IFNa2.

According to the disclosure, an interferon may be a naturally occurring interferon or a functional fragment or variant thereof. An interferon may be human interferon and may be derived from any vertebrate, especially any mammal.

Interleukins (ILs) are a group of cytokines (secreted proteins and signal molecules) that can be divided into four major groups based on distinguishing structural features. However, their amino acid sequence similarity is rather weak (typically 15-25% identity). The human genome encodes more than 50 interleukins and related proteins.

According to the disclosure, an interleukin may be a naturally occurring interleukin or a functional fragment or variant thereof. An interleukin may be human interleukin and may be derived from any vertebrate, especially any mammal.

Cytokines described herein can be prepared as fusion or chimeric polypeptides that include a cytokine portion and a heterologous polypeptide (i.e., a polypeptide that is not a cytokine). The cytokine may be fused to an extended-PK group, which increases circulation half-life. Non-limiting examples of extended-PK groups are described infra. It should be understood that other PK groups that increase the circulation half-life of cytokines, or variants thereof, are also applicable to the present disclosure. In certain embodiments, the extended-PK group is a serum albumin domain (e.g., mouse serum albumin, human serum albumin).

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in U.S. Publication Nos. 2005/0287153 and 2007/0003549). Other exemplary extended-PK groups are disclosed in Kontermann, Expert Opin Biol Ther, 2016 July; 16 (7): 903-15 which is herein incorporated by reference in its entirety. As used herein, an “extended-PK” immunostimulant refers to an immunostimulant moiety in combination with an extended-PK group. In some embodiments, the extended-PK immunostimulant is a fusion protein in which an immunostimulant moiety is linked or fused to an extended-PK group. In certain embodiments, the cytokine can be linked or fused to the FcRn binding domain of the Fc region of an antibody.

In certain embodiments, the serum half-life of an extended-PK cytokine is increased relative to the cytokine alone (i.e., the cytokine not fused to an extended-PK group). In certain embodiments, the serum half-life of the extended-PK cytokine is at least 20, 40, 60, 80, 100, 120, 150, 180, 200, 400, 600, 800, or 1000% longer relative to the serum half-life of the cytokine alone. In certain embodiments, the serum half-life of the extended-PK cytokine is at least 1.5-fold, 2-fold, 2.5-fold, 3-fold, 3.5 fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of the cytokine alone. In certain embodiments, the serum half-life of the extended-PK cytokine is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

As used herein, “half-life” refers to the time taken for the serum or plasma concentration of a compound such as a peptide or polypeptide to reduce by 50%, in vivo, for example due to degradation and/or clearance or sequestration by natural mechanisms. An extended-PK cytokine suitable for use herein is stabilized in vivo and its half-life increased by, e.g., fusion to serum albumin (e.g., HSA or MSA), which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

In certain embodiments, the extended-PK group includes serum albumin, or fragments thereof or variants of the serum albumin or fragments thereof (all of which for the purpose of the present disclosure are comprised by the term “albumin”). Polypeptides described herein may be fused to albumin (or a fragment or variant thereof) to form albumin fusion proteins. Such albumin fusion proteins are described in U.S. Publication No. 20070048282.

As used herein, “albumin fusion protein” refers to a protein formed by the fusion of at least one molecule of albumin (or a fragment or variant thereof) to at least one molecule of a protein such as a cytokine. The albumin fusion protein may be generated by translation of a nucleic acid in which a polynucleotide encoding a cytokine is joined in-frame with a polynucleotide encoding an albumin. The cytokine and albumin, once part of the albumin fusion protein, may each be referred to as a “portion”, “region” or “moiety” of the albumin fusion protein (e.g., a “cytokine portion” or an “albumin protein portion”). In a highly preferred embodiment, an albumin fusion protein comprises at least one molecule of a cytokine (including, but not limited to a mature form of the cytokine) and at least one molecule of albumin (including but not limited to a mature form of albumin). In some embodiments, an albumin fusion protein is processed by a host cell such as a multifunctional cell and secreted into the circulation and/or at a localized site. Processing of the nascent albumin fusion protein that occurs in the secretory pathways of the host cell used for expression of the RNA may include, but is not limited to signal peptide cleavage; formation of disulfide bonds; proper folding; addition and processing of carbohydrates (such as for example, N- and O-linked glycosylation); specific proteolytic cleavages; and/or assembly into multimeric proteins. An albumin fusion protein is preferably encoded by RNA in a non-processed form which in particular has a signal peptide at its N-terminus and following secretion by a cell is preferably present in the processed form wherein in particular the signal peptide has been cleaved off. In a most preferred embodiment, the “processed form of an albumin fusion protein” refers to an albumin fusion protein product which has undergone N-terminal signal peptide cleavage, herein also referred to as a “mature albumin fusion protein”.

In certain embodiments, albumin fusion proteins comprising a cytokine have a higher plasma stability compared to the plasma stability of the same cytokine when not fused to albumin. Plasma stability typically refers to the time period between when the cytokine is administered in vivo and carried into the bloodstream and when the cytokine is degraded and cleared from the bloodstream, into an organ, such as the kidney or liver, that ultimately clears the cytokine from the body. Plasma stability is calculated in terms of the half-life of the cytokine in the bloodstream. The half-life of the cytokine in the bloodstream can be readily determined by common assays known in the art.

As used herein, “albumin” refers collectively to albumin protein or amino acid sequence, or an albumin fragment or variant, having one or more functional activities (e.g., biological activities) of albumin. In particular, “albumin” refers to human albumin or fragments or variants thereof especially the mature form of human albumin, or albumin from other vertebrates or fragments thereof, or variants of these molecules. The albumin may be derived from any vertebrate, especially any mammal, for example human, cow, sheep, or pig. Non-mammalian albumins include, but are not limited to, hen and salmon. The albumin portion of the albumin fusion protein may be from a different animal than the cytokine portion.

In certain embodiments, the albumin is human serum albumin (HSA), or fragments or variants thereof, such as those disclosed in U.S. Pat. No. 5,876,969, WO 2011/124718, WO 2013/075066, and WO 2011/0514789.

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

As used herein, a fragment of albumin sufficient to prolong the therapeutic activity or plasma stability of the cytokine refers to a fragment of albumin sufficient in length or structure to stabilize or prolong the therapeutic activity or plasma stability of the protein so that the plasma stability of the cytokine portion of the albumin fusion protein is prolonged or extended compared to the plasma stability in the non-fusion state.

The albumin portion of the albumin fusion proteins may comprise the full length of the albumin sequence, or may include one or more fragments thereof that are capable of stabilizing or prolonging the therapeutic activity or plasma stability. Such fragments may be of 10 or more amino acids in length or may include about 15, 20, 25, 30, 50, or more contiguous amino acids from the albumin sequence or may include part or all of specific domains of albumin. For instance, one or more fragments of HSA spanning the first two immunoglobulin-like domains may be used. In a preferred embodiment, the HSA fragment is the mature form of HSA.

Generally speaking, an albumin fragment or variant will be at least 100 amino acids long, preferably at least 150 amino acids long.

According to the disclosure, albumin may be naturally occurring albumin or a fragment or variant thereof. Albumin may be human albumin and may be derived from any vertebrate, especially any mammal.

Preferably, the albumin fusion protein comprises albumin as the N-terminal portion, and a cytokine as the C-terminal portion. Alternatively, an albumin fusion protein comprising albumin as the C-terminal portion, and a cytokine as the N-terminal portion may also be used. In other embodiments, the albumin fusion protein has a cytokine fused to both the N-terminus and the C-terminus of albumin. In a preferred embodiment, the cytokines fused at the N- and C-termini are the same cytokines. In another preferred embodiment, the cytokines fused at the N- and C-termini are different cytokines.

In some embodiments, the cytokine(s) is (are) joined to the albumin through (a) peptide linker(s). A linker peptide between the fused portions may provide greater physical separation between the moieties and thus maximize the accessibility of the cytokine portion, for instance, for binding to its cognate receptor. The linker peptide may consist of amino acids such that it is flexible or more rigid. The linker sequence may be cleavable by a protease or chemically.

As used herein, the term “Fc region” refers to the portion of a native immunoglobulin formed by the respective Fc domains (or Fc moieties) of its two heavy chains. As used herein, the term “Fc domain” refers to a portion or fragment of a single immunoglobulin (Ig) heavy chain wherein the Fc domain does not comprise an Fv domain. In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding). For example, the Fc domain lacks the binding domain to FcγR or has reduced binding to FcγR.

The Fc domains of a polypeptide described herein may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

In certain embodiments, an extended-PK group includes an Fc domain or fragments thereof or variants of the Fc domain or fragments thereof (all of which for the purpose of the present disclosure are comprised by the term “Fc domain”). The Fc domain does not contain a variable region that binds to antigen. Fc domains suitable for use in the present disclosure may be obtained from a number of different sources. In certain embodiments, an Fc domain is derived from a human immunoglobulin. In certain embodiments, the Fc domain is from a human IgG1 constant region. It is understood, however, that the Fc domain may be derived from an immunoglobulin of another mammalian species, including for example, a rodent (e.g., a mouse, rat, rabbit, guinea pig) or non-human primate (e.g., chimpanzee, macaque) species.

Moreover, the Fc domain (or a fragment or variant thereof) may be derived from any immunoglobulin class, including IgM, IgG, IgD, IgA, and IgE, and any immunoglobulin isotype, including IgG1, IgG2, IgG3, and IgG4.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g., hinge, CH2, and/or CH3 sequences, or fragments or variants thereof) can be derived from these sequences using art recognized techniques.

In certain embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety. In certain embodiments, the extended-PK group is transferrin, as disclosed in U.S. Pat. Nos. 7,176,278 and 8,158,579, which are herein incorporated by reference in their entirety. In certain embodiments, the extended-PK group is a serum immunoglobulin binding protein such as those disclosed in US2007/0178082, US2014/0220017, and US2017/0145062, which are herein incorporated by reference in their entirety. In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3 (HSA), i.e., a Fn3 protein that binds to human serum albumin.

In certain aspects, the extended-PK cytokine, suitable for use according to the disclosure, can employ one or more peptide linkers. As used herein, the term “peptide linker” refers to a peptide or polypeptide sequence which connects two or more domains (e.g., the extended-PK moiety and a cytokine moiety) in a linear amino acid sequence of a polypeptide chain. For example, peptide linkers may be used to connect a cytokine moiety to a HSA domain.

Linkers suitable for fusing the extended-PK group to e.g. a cytokine are well known in the art. Exemplary linkers include glycine-serine-polypeptide linkers, glycine-proline-polypeptide linkers, and proline-alanine polypeptide linkers. In certain embodiments, the linker is a glycine-serine-polypeptide linker, i.e., a peptide that consists of glycine and serine residues.

### RNA

According to the disclosure a multifunctional cell according to the present disclosure comprises one or more exogenous RNA molecules encoding the immune receptor, e.g., CAR, and two or more cytokines. In an embodiment, the RNA is encoding the CAR and the two or more cytokines, preferably the CAR and two cytokines, i.e., as a single exogenous RNA molecule. In a preferred embodiment the CAR is encoded by an exogenous RNA molecule and the two or more cytokines, preferably two cytokines, are encoded by a different separate exogenous RNA molecule. In a preferred embodiment each, the CAR and each of the two or more cytokines, is encoded by a separate RNA molecule. The RNA molecule described herein can be a linear mRNA, a circular mRNA or a self-amplifying RNA or, in case that at least two separate RNA molecules are used, can be a mixture of mRNA(s) and/or self-amplifying RNA(s).

In some embodiments, the RNA described herein is single-stranded RNA that may be translated into the respective peptide or protein upon entering the multifunctional cells. In addition to wildtype or codon-optimized sequences encoding an amino acid sequence comprising the amino acid sequence of a peptide or protein, such as a CAR or cytokine, the RNA may contain one or more structural elements optimized for maximal efficacy of the RNA with respect to stability and translational efficiency (5′-cap, 5′ UTR, 3′ UTR, poly(A)-tail). In some embodiments, the RNA contains all of these elements. In some embodiments, the RNA molecule is an mRNA containing one or more or all of these elements. In some embodiments, the RNA does not contain a 5′-cap. In some embodiments, the RNA does not contain a 5′ UTR. In some embodiments, the RNA does not contain a 3′ UTR. In some embodiments, the RNA does not contain a poly(A) tail.

In some embodiments, beta-S-ARCA (D1) (m₂^{7,2′-O}GppSpG) or m₂^{7,3′-O}Gppp (m₁^{2′-O}) ApG may be utilized as specific capping structure at the 5′-end of the RNA molecule. As 5′-UTR sequence, the 5′-UTR sequence of the human alpha-globin mRNA, optionally with an optimized ‘Kozak sequence’ to increase translational efficiency may be used. As 3′-UTR sequence, a combination of two sequence elements (FI element) derived from the “amino terminal enhancer of split” (AES) mRNA (called F) and the mitochondrial encoded 12S ribosomal RNA (called I) placed between the coding sequence and the poly(A)-tail to assure higher maximum protein levels and prolonged persistence of the mRNA may be used. These were identified by an ex vivo selection process for sequences that confer RNA stability and augment total protein expression (see WO 2017/060314, herein incorporated by reference). Alternatively, the 3′-UTR may be two re-iterated 3′-UTRs of the human beta-globin mRNA. Furthermore, a poly(A)-tail measuring 110 nucleotides in length, consisting of a stretch of 30 adenosine residues, followed by a 10 nucleotide linker sequence (of random nucleotides) and another 70 adenosine residues may be used. This poly(A)-tail sequence was designed to enhance RNA stability and translational efficiency.

The RNA described herein may encode an amino acid sequence comprising the amino acid sequence of a peptide or protein having biological activity, e.g., an immune receptor such as a CAR or cytokine. The encoded amino acid sequence may comprise amino acid sequences other than the amino acid sequence of a peptide or protein having biological activity. Such other amino acid sequences may support the function or activity of the peptide or protein having biological activity. In some embodiments, such other amino acid sequences comprise an amino acid sequence enhancing antigen processing and/or presentation. Alternatively, or additionally, such other amino acid sequences comprise an amino acid sequence which breaks immunological tolerance.

In some embodiments of the present disclosure, the RNA molecule is “replicon RNA” or “replicon RNA molecule” or simply a “replicon”, in particular “self-replicating RNA” or “self-amplifying RNA” or “replicable RNA molecule”. A replicon RNA molecule is an RNA that is able to be replicated by an RNA-dependent RNA polymerase (replicase) by virtue of comprising nucleotide sequences that can be recognized by the replicase such that the RNA is replicated. The replicon does not necessarily encode the replicase, such that replicons can be replicated in cis (by the encoded replicase; also called a “cis-replicon”) or in trans (by a replicase provided in another manner, e.g., a separate replicase encoding nucleic acid, such as an mRNA; also called “trans-replicon”).

In certain embodiments, the replicon or self-replicating RNA is derived from or comprises elements derived from an ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see José et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e., at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest, e.g., a CAR or cytokine. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

The replicons of the present disclosure are not particle-forming. This means that, following transfection of a cell by a replicon of the present disclosure, the cell does not produce virus particles, such as next generation virus particles. In one embodiment, an RNA replicon is completely free of genetic information encoding any virus structural protein, e.g., alphavirus structural protein, such as core nucleocapsid protein C, envelope protein P62, and/or envelope protein E1. Preferably, the replicon does not comprise a virus packaging signal, e.g., an alphavirus packaging signal. For example, the alphavirus packaging signal comprised in the coding region of nsP2 of SFV (White et al., 1998, J. Virol. 72:4320-4326) may be removed, e.g., by deletion or mutation. A suitable way of removing the alphavirus packaging signal includes adaptation of the codon usage of the coding region of nsP2. The degeneration of the genetic code may allow to delete the function of the packaging signal without affecting the amino acid sequence of the encoded nsP2.

In an embodiment, the RNA described herein may have modified nucleotides/nucleosides/backbone modifications. The term “RNA modification” as used herein may refer to chemical modifications comprising backbone modifications as well as sugar modifications or base modifications. In another embodiment, the RNA does not have any modified nucleotides/nucleosides/backbone modifications.

In this context, a modified RNA molecule as defined herein may contain nucleotide analogues/modifications, e.g., backbone modifications, sugar modifications or base modifications. A backbone modification in connection with the present disclosure is a modification, in which phosphates of the backbone of the nucleotides contained in an RNA molecule as defined herein are chemically modified. A sugar modification in connection with the present disclosure is a chemical modification of the sugar of the nucleotides of the RNA molecule as defined herein. Furthermore, a base modification in connection with the present disclosure is a chemical modification of the base moiety of the nucleotides of the RNA molecule. In this context, nucleotide analogues or modifications are preferably selected from nucleotide analogues, which are applicable for transcription and/or translation.

Sugar Modifications: The modified nucleosides and nucleotides, which may be incorporated into a modified RNA molecule as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R=H, alkyl, cycloalkyl, aryl, aralkyl, heteroaryl or sugar); polyethyleneglycols (PEG),-0 (CH2CH2 0) nCH2CH2 OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy. “Deoxy” modifications include hydrogen, amino (e.g. NH2; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and O. The sugar group can also contain one or more carbons that possess the opposite stereochemical configuration than that of the corresponding carbon in ribose. Thus, a modified RNA molecule can include nucleotides containing, for instance, arabinose as the sugar.

Backbone Modifications: The phosphate backbone may further be modified in the modified nucleosides and nucleotides, which may be incorporated into a modified RNA molecule as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

Base Modifications: The modified nucleosides and nucleotides, which may be incorporated into a modified RNA molecule as described herein can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particular embodiments of the present disclosure, the nucleotide analogues/modifications are selected from base modifications, which are preferably selected from 2-amino-6-chloropurineriboside-5′-triphosphate, 2-aminopurine-riboside-5′-triphosphate; 2-aminoadenosine-5′-triphosphate, 2′-amino-2′-deoxy-cytidine-triphosphate, 2-thiocytidine-5′-triphosphate, 2-thiouridine-5′-triphosphate, 2′-fluorothymidine-5′-triphosphate, 2′-O-methyl inosine-5′-triphosphate 4-thio-uridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-tri-phosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deaza-adenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, N6-methylguanosine-5′-triphosphate, pseudo-uridine-5′-triphosphate, or puromycin-5′-triphosphate, xanthosine-5′-triphosphate. Particular preference may be given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate. In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thiouridine, I-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydro-pseudouridine, 2-thio-dihydrouridine, 2-thio-dihydropseudouridine, 2-methoxy-uridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-I-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diamino-purine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methyl-thio-N6-threonylcarbamoyladenosine, N6,N6-dimethyladenosine, 7-methyladenine, 2-methylthio-adenine, and 2-methoxy-adenine. In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, I-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group. In specific embodiments, a modified nucleoside is 5′-0-(I-thiophosphate)-adenosine, 5′-0-(1-thiophosphate)-cytidine, 5′-0-(I-thiophosphate)-guanosine, 5′-0-(I-thiophosphate)-uridine or 5′-0-(I-thiophosphate)-pseudouridine.

In further embodiments, a modified RNA may comprise nucleoside modifications selected from 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, a-thio-uridine, 4-thio-uridine, 6-aza-uridine, 5-hydroxy-uridine, deoxy-thymidine, 5-methyl-uridine, pyrrolo-cytidine, inosine, a-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, a-thio-adenosine, 8-azido-adenosine, 7-deaza-adenosine.

In certain preferred embodiments, the RNA comprises a modified nucleoside in place of at least one (e.g., every) uridine.

The term “uracil,” as used herein, describes one of the nucleobases that can occur in the nucleic acid of RNA. The structure of uracil is:

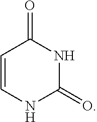

The term “uridine,” as used herein, describes one of the nucleosides that can occur in RNA. The structure of uridine is:

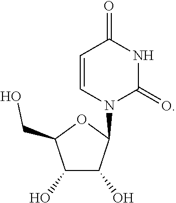 [SMILES: O=c1ccn([C@@H]2O[C@H](CO)[C@@H](O)[C@H]2O)c(=O)[nH]1]

UTP (uridine 5′-triphosphate) has the following structure:

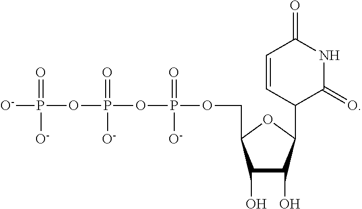

Pseudo-UTP (pseudouridine 5′-triphosphate) has the following structure:

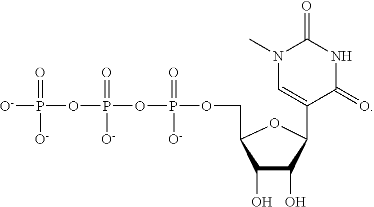

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1ψ), which has the structure:

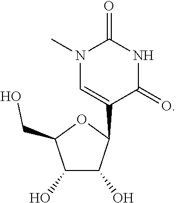 [SMILES: Cn1cc([C@@H]2O[C@H](CO)[C@@H](O)[C@H]2O)c(=O)[nH]c1=O]

N1-methyl-pseudo-UTP has the following structure:

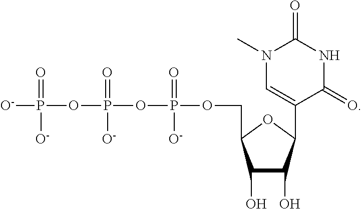

Another exemplary modified nucleoside is 5-methyl-uridine (m5U), which has the structure:

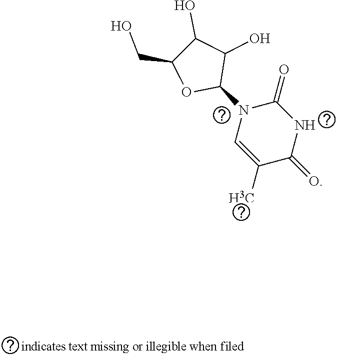 [SMILES: [3CH3]c1cn([C@H]2O[C@@H](CO)C(O)C2O)c(=O)[nH]c1=O]

In certain preferred embodiments, one or more uridine in the RNA described herein is replaced by a modified nucleoside. In some embodiments, the modified nucleoside is a modified uridine.

In certain preferred embodiments, RNA comprises a modified nucleoside in place of at least one uridine. In some embodiments, RNA comprises a modified nucleoside in place of each uridine.

In certain preferred embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In certain preferred embodiments, the modified nucleoside replacing one or more, e.g., all, uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (Tm⁵U), 1-taurinomethyl-pseudouridine, 5-taurinomethyl-2-thio-uridine (τm⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl) pseudouridine (acp³ ψ), 5-(isopentenylaminomethyl) uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino) uridine, or any other modified uridine known in the art.

In certain embodiments, in which the RNA molecule or replicon RNA contains modified U nucleotides, the first and/or second and/or third and/or fourth and/or fifth and/or sixth and/or seventh and/or eighth and/or ninth and/or to tenth U is unmodified.

In an embodiment, the RNA comprises other modified nucleosides or comprises further modified nucleosides, e.g., modified cytidine such as those described above. For example, in one embodiment, in the RNA 5-methylcytidine is substituted partially or completely, preferably completely, for cytidine. In one embodiment, the RNA comprises 5-methylcytidine and one or more selected from pseudouridine (ψ), N1-methyl-pseudouridine (m1ψ), and 5-methyl-uridine (m5U). In an embodiment, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

RNA molecules described herein may optionally be characterized by the following features:

hAg-Kozak: 5′-UTR sequence of the human alpha-globin mRNA with an optimized ‘Kozak sequence’ to increase translational efficiency. One exemplary sequence comprises:

AACUAGUAUUCUUCUGGUCCCCACAGACUCAGAGAGAACCCGCCACC

FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression. One exemplary FI element comprises:

CUGGUACUGCAUGCACGCAAUGCUAGCUGCCCCUUUCCCGUCCUGGGUAC
CCCGAGUCUCCCCCGACCUCGGGUCCCAGGUAUGCUCCCACCUCCACCUG
CCCCACUCACCACCUCUGCUAGUUCCAGACACCUCCCAAGCACGCAGCAA
UGCAGCUCAAAACGCUUAGCCUAGCCACACCCCCACGGGAAACAGCAGUG
AUUAACCUUUAGCAAUAAACGAAAGUUUAACUAAGCUAUACUAACCCCAG
GGUUGGUCAAUUUCGUGCCAGCCACACC

A30L70: A poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells. For example, one such sequence comprises:

AAAAAAAAAAAAAAAAAAAAAAAAAAAAAAGCATATGACTAAAAAAAAAA
AAAAAAAAAAAAAAAAAAAAAAAAAAAAAAAAAAAAAAAAAAAAAAAAAA
AAAAAAAAAA

RNA molecules described herein may optionally be characterized by further features, e.g., by a 5′-cap, a 5′-UTR, a 3′-UTR, a poly(A) sequence, and/or adaptation of the codon usage for optimized translation and/or stabilization of the RNA molecule, as detailed below.

### Cap

In some embodiments, RNA molecules described herein comprise a 5′-cap.

The terms “5′-cap”, “cap”, “5′-cap structure”, “cap structure” are used synonymously to refer to a structure, e.g., a dinucleotide, that is found on the 5′ end of some eukaryotic primary transcripts such as precursor messenger RNA. A 5′-cap is a structure wherein a (optionally modified) guanosine is bonded to the first nucleotide of an mRNA molecule via a 5′ to 5′ triphosphate linkage (or modified triphosphate linkage in the case of certain cap analogs). The terms can refer to a conventional cap or to a cap analog.

“RNA which comprises a 5′-cap” or “RNA which is provided with a 5′-cap” or “RNA which is modified with a 5′-cap” or “capped RNA” refers to RNA which comprises a 5′-cap. For example, providing an RNA with a 5′-cap may be achieved by in vitro transcription of a DNA template in presence of said 5′-cap, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus. In capped RNA, the 3′ position of the first base of a (capped) RNA molecule is linked to the 5′ position of the subsequent base of the RNA molecule (“second base”) via a phosphodiester bond.

In one embodiment, the RNA molecule described herein comprises a 5′-cap. In one embodiment, the RNA molecule described herein does not comprise a 5′-cap.

The term “conventional 5′-cap” refers to a naturally occurring 5′-cap, preferably to the 7-methylguanosine cap. In the 7-methylguanosine cap, the guanosine of the cap is a modified guanosine wherein the modification consists of a methylation at the 7-position.

In the context of the present disclosure, the term “5′-cap analog” refers to a molecular structure that resembles a conventional 5′-cap, but is modified to possess the ability to stabilize RNA if attached thereto, preferably in vivo and/or in a cell. A cap analog is not a conventional 5′-cap.

For the case of eukaryotic mRNA, the 5′-cap has been generally described to be involved in efficient translation of mRNA: in general, in eukaryotes, translation is initiated only at the 5′ end of a messenger RNA (mRNA) molecule, unless an internal ribosomal entry site (IRES) is present. Eukaryotic cells are capable of providing an RNA with a 5′-cap during transcription in the nucleus: newly synthesized mRNAs are usually modified with a 5′-cap structure, e.g.; when the transcript reaches a length of 20 to 30 nucleotides. First, the 5′ terminal nucleotide pppN (ppp representing triphosphate; N representing any nucleoside) is converted in the cell to 5′ GpppN by a capping enzyme having RNA 5′-triphosphatase and guanylyltransferase activities. The GpppN may subsequently be methylated in the cell by a second enzyme with (guanine-7)-methyltransferase activity to form the mono-methylated m⁷GpppN cap. In one embodiment, the 5′-cap used in the present disclosure is a natural 5′-cap.

In the present disclosure, a natural 5′-cap dinucleotide is typically selected from the group consisting of a non-methylated cap dinucleotide (G (5′) ppp (5′) N; also termed GpppN) and a methylated cap dinucleotide ((m⁷G (5′) ppp (5′) N; also termed m⁷GpppN). m⁷GpppN (wherein N is G) is represented by the following formula:

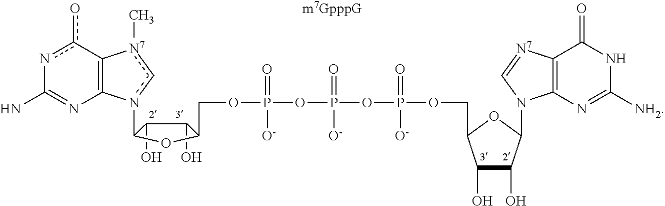

Capped RNA of the present disclosure can be prepared in vitro, and therefore, does not depend on a capping machinery in a host cell. The most frequently used method to make capped RNAs in vitro is to transcribe a DNA template with either a bacterial or bacteriophage RNA polymerase in the presence of all four ribonucleoside triphosphates and a cap dinucleotide such as m⁷G (5′) ppp (5′) G (also called m⁷GpppG). The RNA polymerase initiates transcription with a nucleophilic attack by the 3′-OH of the guanosine moiety of m⁷GpppG on the α-phosphate of the next templated nucleoside triphosphate (pppN), resulting in the intermediate m⁷GpppGpN (wherein N is the second base of the RNA molecule). The formation of the competing GTP-initiated product pppGpN is suppressed by setting the molar ratio of cap to GTP between 5 and 10 during in vitro transcription.

In preferred embodiments of the present disclosure, the 5′-cap (if present) is a 5′-cap analog. These embodiments are particularly suitable if the RNA is obtained by in vitro transcription, e.g. is an in vitro transcribed RNA (IVT-RNA). Cap analogs have been initially described to facilitate large scale synthesis of RNA transcripts by means of in vitro transcription.

For messenger RNA, some cap analogs (synthetic caps) have been generally described to date, and they can all be used in the context of the present disclosure. Ideally, a cap analog is selected that is associated with higher translation efficiency and/or increased resistance to in vivo degradation and/or increased resistance to in vitro degradation.

Preferably, a cap analog is used that can only be incorporated into an RNA chain in one orientation. Pasquinelli et al., 1995, RNA J. 1:957-967) demonstrated that during in vitro transcription, bacteriophage RNA polymerases use the 7-methylguanosine unit for initiation of transcription, whereby around 40-50% of the transcripts with cap possess the cap dinucleotide in a reverse orientation (i.e., the initial reaction product is Gpppm⁷GpN). Compared to the RNAs with a correct cap, RNAs with a reverse cap are not functional with respect to translation of a nucleic acid sequence into protein. Thus, it is desirable to incorporate the cap in the correct orientation, i.e., resulting in an RNA with a structure essentially corresponding to m⁷GpppGpN etc. It has been shown that the reverse integration of the cap-dinucleotide is inhibited by the substitution of either the 2′- or the 3′—OH group of the methylated guanosine unit (Stepinski et al., 2001, RNA J. 7:1486-1495; Peng et al., 2002, Org. Lett. 24:161-164).

RNAs which are synthesized in presence of such “anti reverse cap analogs” are translated more efficiently than RNAs which are in vitro transcribed in presence of the conventional 5′-cap m7GpppG. To that end, one cap analog in which the 3′ OH group of the methylated guanosine unit is replaced by OCH₃ is described, e.g., by Holtkamp et al., 2006, Blood 108:4009-4017 (7-methyl (3′-O-methyl) GpppG; anti-reverse cap analog (ARCA)). ARCA is a suitable cap dinucleotide according to the present disclosure.

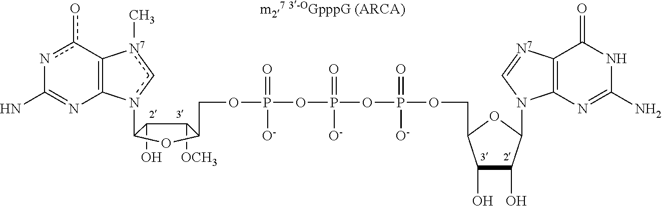

In an embodiment, the RNA molecule described herein is essentially not susceptible to decapping. This is important because, in general, the amount of protein produced from synthetic mRNAs introduced into cultured mammalian cells is limited by the natural degradation of mRNA. One in vivo pathway for mRNA degradation begins with the removal of the mRNA cap. This removal is catalyzed by a heterodimeric pyrophosphatase, which contains a regulatory subunit (Dcp1) and a catalytic subunit (Dcp2). The catalytic subunit cleaves between the α and β phosphate groups of the triphosphate bridge. In the present disclosure, a cap analog may be selected or present that is not susceptible, or less susceptible, to that type of cleavage. A suitable cap analog for this purpose may be selected from a cap dinucleotide according to formula (I):

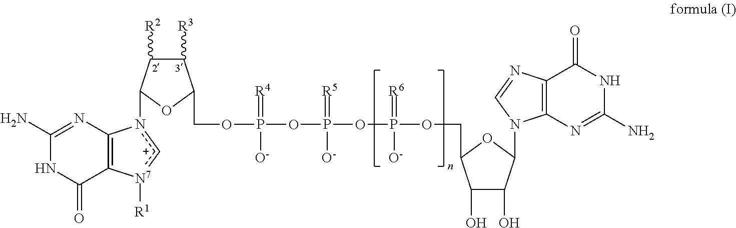

wherein R¹ is selected from the group consisting of optionally substituted alkyl, optionally substituted alkenyl, optionally substituted alkynyl, optionally substituted cycloalkyl, optionally substituted heterocyclyl, optionally substituted aryl, and optionally substituted heteroaryl,
R² and R³ are independently selected from the group consisting of H, halo, OH, and optionally substituted alkoxy, or R² and R³ together form O—X—O, wherein X is selected from the group consisting of optionally substituted CH₂, CH₂CH₂, CH₂CH₂CH₂, CH₂CH(CH₃), and
C(CH₃)₂, or R² is combined with the hydrogen atom at position 4′ of the ring to which R² is attached to form —O—CH₂— or —CH₂—O—,
R⁵ is selected from the group consisting of S, Se, and BH₃,
R⁴ and R⁶ are independently selected from the group consisting of O, S, Se, and BH₃,
n is 1, 2, or 3.

Preferred embodiments for R¹, R², R3, R⁴, R⁵, R⁶ are disclosed in WO 2011/015347 A1 and may be selected accordingly in the present disclosure.

For example, in an embodiment, the RNA molecule described herein comprises a phosphorothioate-cap-analog. Phosphorothioate-cap-analogs are specific cap analogs in which one of the three non-bridging O atoms in the triphosphate chain is replaced with an S atom, i.e., one of R⁴, R⁵ or R⁶ in Formula (I) is S. Phosphorothioate-cap-analogs have been described by Kowalska et al., 2008, RNA, 14:1119-1131, as a solution to the undesired decapping process, and thus to increase the stability of RNA in vivo. In particular, the substitution of an oxygen atom for a sulphur atom at the beta-phosphate group of the 5′-cap results in stabilization against Dcp2. In that embodiment, which is preferred in the present disclosure, R⁵ in Formula (I) is S; and R⁴ and R⁶ are O.

In a further embodiment, the RNA molecule described herein comprises a phosphorothioate-cap-analog wherein the phosphorothioate modification of the RNA 5′-cap is combined with an “anti-reverse cap analog” (ARCA) modification. Respective ARCA-phosphorothioate-cap-analogs are described in WO 2008/157688 A2, and they can all be used in the RNA molecule of the present disclosure. In that embodiment, at least one of R² or R³ in Formula (I) is not OH, preferably one among R² and R³ is methoxy (OCH₃), and the other one among R² and R³ is preferably OH. In a preferred embodiment, an oxygen atom is substituted for a sulphur atom at the beta-phosphate group (so that R⁵ in Formula (I) is S; and R⁴ and R⁶ are O). It is believed that the phosphorothioate modification of the ARCA ensures that the α, β, and γ phosphorothioate groups are precisely positioned within the active sites of cap-binding proteins in both the translational and decapping machinery. At least some of these analogs are essentially resistant to pyrophosphatase Dcp1/Dcp2. Phosphorothioate-modified ARCAs were described to have a much higher affinity for eIF4E than the corresponding ARCAs lacking a phosphorothioate group.

A respective cap analog that is particularly preferred in the present disclosure, i.e., m₂^{7,2′-O}GppspG, is termed beta-S-ARCA (WO 2008/157688 A2; Kuhn et al., 2010, Gene Ther. 17:961-971). Thus, in one embodiment of the present disclosure, the RNA described herein is modified with beta-S-ARCA. beta-S-ARCA is represented by the following structure:

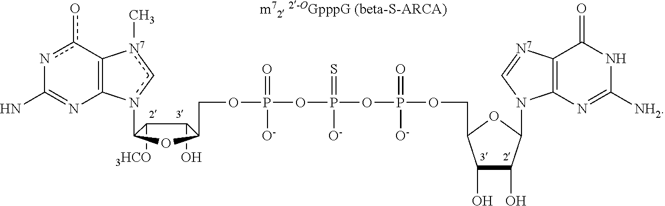

In general, the replacement of an oxygen atom for a sulphur atom at a bridging phosphate results in phosphorothioate diastereomers which are designated D1 and D2, based on their elution pattern in HPLC. Briefly, the D1 diastereomer of beta-S-ARCA″ or “beta-S-ARCA (D1)” is the diastereomer of beta-S-ARCA which elutes first on an HPLC column compared to the D2 diastereomer of beta-S-ARCA (beta-S-ARCA (D2)) and thus exhibits a shorter retention time. Determination of the stereochemical configuration by HPLC is described in WO 2011/015347 A1.

In a first particularly preferred embodiment of the present disclosure, RNA molecule described herein is modified with the beta-S-ARCA (D2) diastereomer. The two diastereomers of beta-S-ARCA differ in sensitivity against nucleases. It has been shown that RNA carrying the D2 diastereomer of beta-S-ARCA is almost fully resistant against Dcp2 cleavage (only 6% cleavage compared to RNA which has been synthesized in presence of the unmodified ARCA 5′-cap), whereas RNA with the beta-S-ARCA (D1) 5′-cap exhibits an intermediary sensitivity to Dcp2 cleavage (71% cleavage). It has further been shown that the increased stability against Dcp2 cleavage correlates with increased protein expression in mammalian cells. In particular, it has been shown that RNAs carrying the beta-S-ARCA (D2) cap are more efficiently translated in mammalian cells than RNAs carrying the beta-S-ARCA (D1) cap. Therefore, in one embodiment of the present disclosure, RNA molecule described herein is modified with a cap analog according to Formula (I), characterized by a stereochemical configuration at the P atom comprising the substituent R⁵ in Formula (I) that corresponds to that at the PB atom of the D2 diastereomer of beta-S-ARCA. In that embodiment, R⁵ in Formula (I) is S; and R⁴ and R⁶ are O. Additionally, at least one of R² or R³ in Formula (I) is preferably not OH, preferably one among R² and R³ is methoxy (OCH₃), and the other one among R² and R³ is preferably OH.

In a second particularly preferred embodiment, the RNA molecule described herein is modified with the beta-S-ARCA (D1) diastereomer. This embodiment is particularly suitable for transfer of capped RNA into immature antigen presenting cells. It has been demonstrated that the beta-S-ARCA (D1) diastereomer, upon transfer of respectively capped RNA into immature antigen presenting cells, is particularly suitable for increasing the stability of the RNA, increasing translation efficiency of the RNA, prolonging translation of the RNA, increasing total protein expression of the RNA, and/or increasing the immune response against an antigen or antigen peptide encoded by said RNA (Kuhn et al., 2010, Gene Ther. 17:961-971). Therefore, in an alternative embodiment of the present disclosure, the RNA molecule described herein is modified with a cap analog according to Formula (I), characterized by a stereochemical configuration at the P atom comprising the substituent R⁵ in Formula (I) that corresponds to that at the PB atom of the D1 diastereomer of beta-S-ARCA. Respective cap analogs and embodiments thereof are described in WO 2011/015347 A1 and Kuhn et al., 2010, Gene Ther. 17:961-971. Any cap analog described in WO 2011/015347 A1, wherein the stereochemical configuration at the P atom comprising the substituent R⁵ corresponds to that at the PB atom of the D1 diastereomer of beta-S-ARCA, may be used in the present disclosure. Preferably, R⁵ in Formula (I) is S; and R⁴ and R⁶ are O. Additionally, at least one of R² or R³ in Formula (I) is preferably not OH, preferably one among R² and R³ is methoxy (OCH3), and the other one among R² and R³ is preferably OH.

In one embodiment, the RNA molecule described herein is modified with a 5′-cap structure according to Formula (I) wherein any one phosphate group is replaced by a boranophosphate group or a phosphoroselenoate group. Such caps have increased stability both in vitro and in vivo. Optionally, the respective compound has a 2′-O— or 3′-O-alkyl group (wherein alkyl is preferably methyl); respective cap analogs are termed BH₃-ARCAs or Se-ARCAs. Compounds that are particularly suitable for capping of mRNA include the β-BH₃-ARCAs and β-Se-ARCAS, as described in WO 2009/149253 A2. For these compounds, a stereochemical configuration at the P atom comprising the substituent R⁵ in Formula (I) that corresponds to that at the PB atom of the D1 diastereomer of beta-S-ARCA is preferred.

In one embodiment, the 5′ cap can be a CleanCap supplied by Trilink Biotechnologies, San Diego, CA having the following structure:

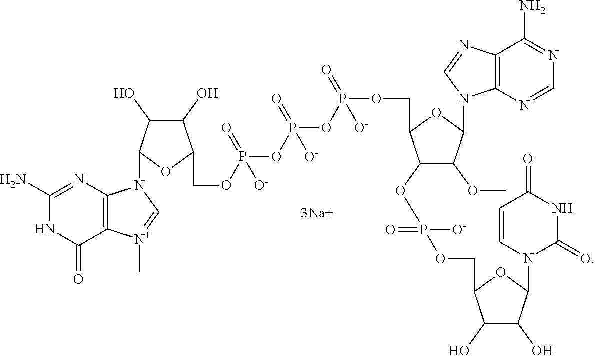 [SMILES: COC1C(OP(=O)([O-])OCC2OC(n3ccc(=O)[nH]c3=O)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

### UTR

The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR).

A 3′-UTR, if present, is located at the 3′ end of a gene, downstream of the termination codon of a protein-encoding region, but the term “3′-UTR” does preferably not include the poly(A) tail. Thus, the 3′-UTR is upstream of the poly(A) sitail (if present), e.g. directly adjacent to the poly(A) tail.

A 5′-UTR, if present, is located at the 5′ end of a gene, upstream of the start codon of a protein-encoding region. A 5′-UTR is downstream of the 5′-cap (if present), e.g. directly adjacent to the 5′-cap.

5′- and/or 3′-untranslated regions may, according to the disclosure, be functionally linked to an open reading frame, so as for these regions to be associated with the open reading frame in such a way that the stability and/or translation efficiency of the RNA comprising said open reading frame are increased.

In some embodiments, the RNA molecule according to the present disclosure comprises a 5′-UTR and/or a 3′-UTR.

UTRs are implicated in stability and translation efficiency of RNA. Both can be improved, besides structural modifications concerning the 5′-cap and/or the 3′ poly(A)-tail as described herein, by selecting specific 5′ and/or 3′ untranslated regions (UTRs). Sequence elements within the UTRs are generally understood to influence translational efficiency (mainly 5′-UTR) and RNA stability (mainly 3′-UTR). It is preferable that a 5′-UTR is present that is active in order to increase the translation efficiency and/or stability of the RNA molecule. Independently or additionally, it is preferable that a 3′-UTR is present that is active in order to increase the translation efficiency and/or stability of the RNA molecule.

The terms “active in order to increase the translation efficiency” and/or “active in order to increase the stability”, with reference to a first nucleic acid sequence (e.g. a UTR), means that the first nucleic acid sequence is capable of modifying, in a common transcript with a second nucleic acid sequence, the translation efficiency and/or stability of said second nucleic acid sequence in such a way that said translation efficiency and/or stability is increased in comparison with the translation efficiency and/or stability of said second nucleic acid sequence in the absence of said first nucleic acid sequence.

In one embodiment, the RNA molecule comprises a 5′-UTR derived from a eukaryotic 5′-UTR and/or a 3′-UTR derived from a eukaryotic 3′-UTR.

A 5′-UTR according to the present disclosure can comprise any combination of more than one nucleic acid sequence, optionally separated by a linker. A 3′-UTR according to the present disclosure can comprise any combination of more than one nucleic acid sequence, optionally separated by a linker.

The term “linker” according to the disclosure relates to a nucleic acid sequence added between two nucleic acid sequences to connect said two nucleic acid sequences. There is no particular limitation regarding the linker sequence.

A 3′-UTR typically has a length of 200 to 2000 nucleotides, e.g. 500 to 1500 nucleotides. The 3′-untranslated regions of immunoglobulin mRNAs are relatively short (fewer than about 300 nucleotides), while the 3′-untranslated regions of other genes are relatively long. For example, the 3′-untranslated region of tPA is about 800 nucleotides in length, that of factor VIII is about 1800 nucleotides in length and that of erythropoietin is about 560 nucleotides in length. The 3′-untranslated regions of mammalian mRNA typically have a homology region known as the AAUAAA hexanucleotide sequence. This sequence is presumably the poly(A) siattachment signal and is frequently located from 10 to 30 bases upstream of the poly(A) siattachment site. 3′-untranslated regions may contain one or more inverted repeats which can fold to give stem-loop structures which act as barriers for exoribonucleases or interact with proteins known to increase RNA stability (e.g. RNA-binding proteins).

The human beta-globin 3′-UTR, particularly two consecutive identical copies of the human beta-globin 3′-UTR, contributes to high transcript stability and translational efficiency (Holtkamp et al., 2006, Blood 108:4009-4017). Thus, in one embodiment, the RNA molecule described herein comprises two consecutive identical copies of the human beta-globin 3′-UTR. Thus, it comprises in the 5′→3′ direction: (a) optionally a 5′-UTR; (b) an open reading frame; (c) a 3′-UTR; said 3′-UTR comprising two consecutive identical copies of the human beta-globin 3′-UTR, a fragment thereof, or a variant of the human beta-globin 3′-UTR or fragment thereof.

In an embodiment, the RNA molecule described herein comprises a 3′-UTR which is active in order to increase translation efficiency and/or stability, but which is not the human beta-globin 3′-UTR, a fragment thereof, or a variant of the human beta-globin 3′-UTR or fragment thereof.

In an embodiment, the RNA molecule described herein comprises a 5′-UTR which is active in order to increase translation efficiency and/or stability.

### Poly(A) Sequence

In some embodiments, the RNA molecule described herein comprises a 3′-poly(A) sequence. The terms “3′-poly(A) sequence”, “poly(A) sequence”, “poly(A)-tail” and “polyA structure” are used synonymously throughout this application.

In one embodiment, a poly(A) sequence comprises or essentially consists of or consists of at least 20, preferably at least 26, preferably at least 40, preferably at least 80, preferably at least 100 and preferably up to 500, preferably up to 400, preferably up to 300, preferably up to 200, and in particular up to 150, A nucleotides, and in particular about 120 A nucleotides. In this context “essentially consists of” means that most nucleotides in the poly(A) sequence, typically at least 50%, and preferably at least 75% by number of nucleotides in the “poly(A) sequence”, are A nucleotides (adenylate), but permits that remaining nucleotides are nucleotides other than A nucleotides, such as U nucleotides (uridylate), G nucleotides (guanylate), C nucleotides (cytidylate). In this context “consists of” means that all nucleotides in the poly(A) sequence, i.e. 100% by number of nucleotides in the poly(A) sequence, are A nucleotides. The term “A nucleotide” or “A” refers to adenylate.

Indeed, it has been demonstrated that a 3′ poly(A) sequence of about 120 A nucleotides has a beneficial influence on the levels of RNA in transfected eukaryotic cells, as well as on the levels of protein that is translated from an open reading frame that is present upstream (5′) of the 3′ poly(A) sequence (Holtkamp et al., 2006, Blood, vol. 108, pp. 4009-4017).

In an embodiment, a 3′ poly(A) sequence can be attached during RNA transcription, i.e. during preparation of in vitro transcribed RNA, based on a DNA template comprising repeated dT nucleotides (deoxythymidylate) in the strand complementary to the coding strand. The DNA sequence encoding a poly(A) sequence (coding strand) is referred to as poly(A) cassette.

In an embodiment, the 3′ poly(A) cassette present in the coding strand of DNA essentially consists of dA nucleotides, but is interrupted by a random sequence having an equal distribution of the four nucleotides (dA, dC, dG, dT). Such random sequence may be 5 to 50, preferably 10 to 30, more preferably 10 to 20 nucleotides in length. Such a cassette is disclosed in WO 2016/005004 A1. Any poly(A) cassette disclosed in WO 2016/005004 A1 may be used in the present disclosure. A poly(A) cassette that essentially consists of dA nucleotides, but is interrupted by a random sequence having an equal distribution of the four nucleotides (dA, dC, dG, dT) and having a length of, e.g., 5 to 50 nucleotides shows, on DNA level, constant propagation of plasmid DNA in E. coli and is still associated, on RNA level, with the beneficial properties with respect to supporting RNA stability and translational efficiency.

Consequently, in a preferred embodiment of the present disclosure, the 3′ poly(A) sequence contained in an RNA molecule described herein essentially consists of A nucleotides, but is interrupted by a random sequence having an equal distribution of the four nucleotides (A, C, G, U). Such random sequence may be 5 to 50, preferably 10 to 30, more preferably 10 to 20 nucleotides in length.

### Codon Usage

In general, the degeneracy of the genetic code will allow the substitution of certain codons (base triplets coding for an amino acid) that are present in an RNA sequence by other codons (base triplets), while maintaining the same coding capacity (so that the replacing codon encodes the same amino acid as the replaced codon). In some embodiments of the present disclosure, at least one codon of an open reading frame comprised by an RNA molecule differs from the respective codon in the respective open reading frame in the species from which the open reading frame originates. In that embodiment, the coding sequence of the open reading frame is said to be “adapted” or “modified”. The coding sequence of an open reading frame comprised by the RNA molecule may be adapted.

For example, when the coding sequence of an open reading frame is adapted, frequently used codons may be selected: WO 2009/024567 A1 describes the adaptation of a coding sequence of a nucleic acid molecule, involving the substitution of rare codons by more frequently used codons. Since the frequency of codon usage depends on the host cell or host organism, that type of adaptation is suitable to fit a nucleic acid sequence to expression in a particular host cell or host organism. Generally, speaking, more frequently used codons are typically translated more efficiently in a host cell or host organism, although adaptation of all codons of an open reading frame is not always required.

For example, when the coding sequence of an open reading frame is adapted, the content of G (guanylate) residues and C (cytidylate) residues may be altered by selecting codons with the highest GC-rich content for each amino acid. RNA molecules with GC-rich open reading frames were reported to have the potential to reduce immune activation and to improve translation and half-life of RNA (Thess et al., 2015, Mol. Ther. 23:1457-1465).

### DNA

The present disclosure also provides a DNA comprising a nucleic acid sequence encoding one or more RNA molecules according to the present disclosure.

Preferably, the DNA is double-stranded.

In a preferred embodiment, the DNA is a plasmid. The term “plasmid”, as used herein, generally relates to a construct of extrachromosomal genetic material, usually a circular DNA duplex, which can replicate independently of chromosomal DNA.

The DNA of the present disclosure may comprise a promoter that can be recognized by a DNA-dependent RNA-polymerase. This allows for transcription of the encoded RNA in vivo or in vitro, e.g. of the RNA of the present disclosure. IVT vectors may be used in a standardized manner as template for in vitro transcription. Examples of promoters preferred according to the disclosure are promoters for SP6, T3 or T7 polymerase.

In one embodiment, the DNA of the present disclosure is an isolated nucleic acid molecule.

### Methods of Preparing RNA

The RNA molecules according to the present disclosure may be obtainable by in vitro transcription. In vitro-transcribed RNA (IVT-RNA) is of particular interest in the present disclosure. IVT-RNA is obtainable by transcription from a nucleic acid molecule (particularly a DNA molecule). The DNA molecule(s) of the present disclosure are suitable for such purposes, particularly if comprising a promoter that can be recognized by a DNA-dependent RNA-polymerase.

RNA according to the present disclosure can be synthesized in vitro. This allows to add cap-analogs to the in vitro transcription reaction. Typically, the poly(A) sitail is encoded by a poly-(dT) sequence on the DNA template. Alternatively, capping and poly(A) tail addition can be achieved enzymatically after transcription.

The in vitro transcription methodology is known to the skilled person. For example, as mentioned in WO 2011/015347 A1, a variety of in vitro transcription kits is commercially available.

In some embodiments, the RNA molecule or RNA, e.g., RNA encoding a CAR and/or two or more cytokines, used in the present disclosure is non-immunogenic. The RNA molecule may be standard RNA or non-immunogenic RNA.

The term “non-immunogenic RNA” (such as “non-immunogenic mRNA”) as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In certain embodiments, non-immunogenic RNA, which is also termed modified RNA (modRNA) herein, is rendered non-immunogenic by incorporating modified nucleosides suppressing RNA-mediated activation of innate immune receptors into the RNA and/or limiting the amount of double-stranded RNA (dsRNA), e.g., by limiting the formation of double-stranded RNA (dsRNA), e.g., during in vitro transcription, and/or by removing double-stranded RNA (dsRNA), e.g., following in vitro transcription. In certain embodiments, non-immunogenic RNA is rendered non-immunogenic by incorporating modified nucleosides suppressing RNA-mediated activation of innate immune receptors into the RNA and/or by removing double-stranded RNA (dsRNA), e.g., following in vitro transcription.

For rendering the non-immunogenic RNA (especially mRNA) non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In some embodiments, the modified nucleosides comprise a replacement of one or more uridines with a nucleoside comprising a modified nucleobase. In some embodiments, the modified nucleobase is a modified uracil. In some embodiments, the nucleoside comprising a modified nucleobase is selected from the group consisting of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl) pseudouridine (acp³ ω), 5-(isopentenylaminomethyl) uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine. In certain embodiments, the nucleoside comprising a modified nucleobase is pseudouridine (ψ), N1-methyl-pseudouridine (m1ψ) or 5-methyl-uridine (m5U), in particular N1-methyl-pseudouridine.

In some embodiments, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines.

During synthesis of mRNA by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. Formation of dsRNA can be limited during synthesis of mRNA by in vitro transcription (IVT), for example, by limiting the amount of uridine triphosphate (UTP) during synthesis. Optionally, UTP may be added once or several times during synthesis of mRNA. Also, dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In some embodiments, an RNA preparation is contacted with a cellulose material and the ssRNA is separated from the cellulose material under conditions which allow binding of dsRNA to the cellulose material and do not allow binding of ssRNA to the cellulose material. Suitable methods for providing ssRNA are disclosed, for example, in WO 2017/182524.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In some embodiments, the amount of double-stranded RNA (dsRNA) is limited, e.g., dsRNA (especially mRNA) is removed from non-immunogenic RNA, such that less than 10%, less than 5%, less than 4%, less than 3%, less than 2%, less than 1%, less than 0.5%, less than 0.3%, less than 0.1%, less than 0.05%, less than 0.03%, less than 0.01%, less than 0.005%, less than 0.004%, less than 0.003%, less than 0.002%, less than 0.001%, or less than 0.0005% of the RNA in the non-immunogenic RNA composition is dsRNA. In some embodiments, the non-immunogenic RNA (especially mRNA) is free or essentially free of dsRNA. In some embodiments, the non-immunogenic RNA (especially mRNA) composition comprises a purified preparation of single-stranded nucleoside modified RNA. In some embodiments, the non-immunogenic RNA (especially mRNA) composition comprises single-stranded nucleoside modified RNA (especially mRNA) and is substantially free of double stranded RNA (dsRNA). In some embodiments, the non-immunogenic RNA (especially mRNA) composition comprises at least 90%, at least 91%, at least 92%, at least 93%, at least 94%, at least 95%, at least 96%, at least 97%, at least 98%, at least 99%, at least 99.5%, at least 99.9%, at least 99.99%, at least 99.991%, at least 99.992%, at least 99.993%, at least 99.994%, at least 99.995%, at least 99.996%, at least 99.997%, or at least 99.998% single stranded nucleoside modified RNA, relative to all other nucleic acid molecules (DNA, dsRNA, etc.).

Various methods can be used to determine the amount of dsRNA. For example, a sample may be contacted with dsRNA-specific antibody and the amount of antibody binding to RNA may be taken as a measure for the amount of dsRNA in the sample. A sample containing a known amount of dsRNA may be used as a reference.

For example, RNA may be spotted onto a membrane, e.g., nylon blotting membrane. The membrane may be blocked, e.g., in TBS-T buffer (20 mM TRIS pH 7.4, 137 mM NaCl, 0.1% (v/v) TWEEN-20) containing 5% (w/v) skim milk powder. For detection of dsRNA, the membrane may be incubated with dsRNA-specific antibody, e.g., dsRNA-specific mouse mAb (English & Scientific Consulting, Szirák, Hungary). After washing, e.g., with TBS-T, the membrane may be incubated with a secondary antibody, e.g., HRP-conjugated donkey anti-mouse IgG (Jackson ImmunoResearch, Cat #715-035-150), and the signal provided by the secondary antibody may be detected.

In some embodiments, the non-immunogenic RNA (especially mRNA) is translated in a cell more efficiently than standard RNA with the same sequence. In some embodiments, translation is enhanced by a factor of 2-fold relative to its unmodified counterpart. In some embodiments, translation is enhanced by a 3-fold factor. In some embodiments, translation is enhanced by a 4-fold factor. In some embodiments, translation is enhanced by a 5-fold factor. In some embodiments, translation is enhanced by a 6-fold factor. In some embodiments, translation is enhanced by a 7-fold factor. In some embodiments, translation is enhanced by an 8-fold factor. In some embodiments, translation is enhanced by a 9-fold factor. In some embodiments, translation is enhanced by a 10-fold factor. In some embodiments, translation is enhanced by a 15-fold factor. In some embodiments, translation is enhanced by a 20-fold factor. In some embodiments, translation is enhanced by a 50-fold factor. In some embodiments, translation is enhanced by a 100-fold factor. In some embodiments, translation is enhanced by a 200-fold factor. In some embodiments, translation is enhanced by a 500-fold factor. In some embodiments, translation is enhanced by a 1000-fold factor. In some embodiments, translation is enhanced by a 2000-fold factor. In some embodiments, the factor is 10-1000-fold. In some embodiments, the factor is 10-100-fold. In some embodiments, the factor is 10-200-fold. In some embodiments, the factor is 10-300-fold. In some embodiments, the factor is 10-500-fold. In some embodiments, the factor is 20-1000-fold. In some embodiments, the factor is 30-1000-fold. In some embodiments, the factor is 50-1000-fold. In some embodiments, the factor is 100-1000-fold. In some embodiments, the factor is 200-1000-fold. In some embodiments, translation is enhanced by any other significant amount or range of amounts.

In some embodiments, the non-immunogenic RNA (especially mRNA) exhibits significantly less innate immunogenicity than standard RNA with the same sequence. In some embodiments, the non-immunogenic RNA (especially mRNA) exhibits an innate immune response that is 2-fold less than its unmodified counterpart. In some embodiments, innate immunogenicity is reduced by a 3-fold factor. In some embodiments, innate immunogenicity is reduced by a 4-fold factor. In some embodiments, innate immunogenicity is reduced by a 5-fold factor. In some embodiments, innate immunogenicity is reduced by a 6-fold factor. In some embodiments, innate immunogenicity is reduced by a 7-fold factor. In some embodiments, innate immunogenicity is reduced by a 8-fold factor. In some embodiments, innate immunogenicity is reduced by a 9-fold factor. In some embodiments, innate immunogenicity is reduced by a 10-fold factor. In some embodiments, innate immunogenicity is reduced by a 15-fold factor. In some embodiments, innate immunogenicity is reduced by a 20-fold factor. In some embodiments, innate immunogenicity is reduced by a 50-fold factor. In some embodiments, innate immunogenicity is reduced by a 100-fold factor. In some embodiments, innate immunogenicity is reduced by a 200-fold factor. In some embodiments, innate immunogenicity is reduced by a 500-fold factor. In some embodiments, innate immunogenicity is reduced by a 1000-fold factor. In some embodiments, innate immunogenicity is reduced by a 2000-fold factor.

The term “exhibits significantly less innate immunogenicity” refers to a detectable decrease in innate immunogenicity. In some embodiments, the term refers to a decrease such that an effective amount of the non-immunogenic RNA (especially mRNA) can be administered without triggering a detectable innate immune response. In some embodiments, the term refers to a decrease such that the non-immunogenic RNA (especially mRNA) can be repeatedly administered without eliciting an innate immune response sufficient to detectably reduce production of the protein encoded by the non-immunogenic RNA. In some embodiments, the decrease is such that the non-immunogenic RNA (especially mRNA) can be repeatedly administered without eliciting an innate immune response sufficient to eliminate detectable production of the protein encoded by the non-immunogenic RNA.

“Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

Multifunctional Cells and/or RNA for Use in a Method of Treatment

In an aspect of the disclosure the multifunctional cells, preferably T cells, according to the disclosure are for use in a method of treating a disease or disorder, preferably cancer. Any of the multifunctional cells comprising one or more RNA molecules encoding an immune receptor, such as a chimeric antigen receptor, and two or more cytokines previously described can be used in such a method. In another aspect, the multifunctional cells, preferably T cells, according to the disclosure are for use in a method of treating a disease or disorder, preferably cancer. Any of the multifunctional cells comprising one or more RNA molecules encoding an immune receptor, such as a chimeric antigen receptor, and one or more cytokines, preferably two or more cytokines, previously described can be used in such a method. Preferably, the one cytokine is IL-12 and the multifunctional cell does not contain any exogenous DNA encoding the cytokine and/or encoding the cytokine and the immune receptor.

In an aspect of the disclosure, a method is provided for treating a disease or disorder in a patient comprising administering to the patient a multifunctional cell transiently expressing an immune receptor and two or more cytokines, wherein the disease or disorder is characterized by the expression of an antigen to which the immune receptor binds, wherein the cell comprises one or more exogenous RNA molecules encoding the immune receptor and the two or more cytokines, preferably wherein the patient is a human.

In an aspect of the disclosure, one or more exogenous RNA molecules encoding an immune receptor and two or more cytokines are for use in a method of treating a disease or disorder in a patient, said method comprising administering to the patient the one or more exogenous RNA molecules, wherein the disease or disorder is characterized by the expression of an antigen to which the immune receptor binds, preferably wherein the patient is a human.

In an aspect of the disclosure, a method is provided for treating a disease or disorder in a patient comprising administering to the patient one or more exogenous RNA molecules encoding an immune receptor and two or more cytokines, wherein the disease or disorder is characterized by the expression of an antigen to which the immune receptor binds, preferably wherein the patient is a human.

In an aspect of the disclosure, a method is provided for treating a disease or disorder in a patient comprising administering to the patient one or more exogenous RNA molecules encoding an immune receptor and one or more cytokines, wherein the disease or disorder is characterized by the expression of an antigen to which the immune receptor binds, preferably wherein the patient is a human, preferably wherein the cytokine is IL-12 and administration is not intra-tumorally.

In an aspect of the disclosure, one or more exogenous RNA molecules encoding an immune receptor and one or more cytokines is provided for use in a method for treating a disease or disorder in a patient comprising administering to the patient one or more exogenous RNA molecules encoding an immune receptor and one or more cytokines, wherein the disease or disorder is characterized by the expression of an antigen to which the immune receptor binds, preferably wherein the patient is a human, preferably wherein the cytokine is IL-12 and administration is not intra-tumorally.

In a preferred embodiment, the disease or disorder to be treated is a glioblastoma. Preferably the tumor cells of the glioblastoma are expressing a ligand of NKG2D receptor.

An exemplary aspect of the disclosure is a multifunctional cell, preferably a cytotoxic cell, expressing a chimeric antigen receptor (CAR), interleukin-12, and interferon-α2 for use in a method of treating a disease or disorder, preferably glioblastoma, wherein the multifunctional cell comprises three exogenous mRNA molecules, each individually encoding the CAR, interleukin-12, and interferon-α2, wherein the disease or disorder is characterized by expression of an antigen that is bound by the CAR.

A further exemplary aspect of the disclosure is a multifunctional cell, preferably a cytotoxic cell, expressing a chimeric antigen receptor (CAR) capable of binding to a glioblastoma specific antigen, interleukin-12, and interferon-α2 for use in a method of treating glioblastoma, wherein the multifunctional cell comprises three exogenous mRNA molecules, each individually encoding the CAR, interleukin-12, and interferon-α2, wherein the CAR is able to bind to an antigen expressed by the glioblastoma.

A further exemplary aspect of the disclosure is a multifunctional cell, preferably a cytotoxic cell, expressing a chimeric antigen receptor (CAR) capable of binding to a NKG2D ligand, and two or more cytokines for use in a method of treating glioblastoma, wherein the multifunctional cell comprises three exogenous mRNA molecules, each individually encoding the CAR, interleukin-12, and interferon-α2.

In an embodiment, the cell does not contain any exogenous DNA molecules encoding the CAR and/or the cytokines.

A further exemplary aspect of the disclosure is a multifunctional cell, preferably a cytotoxic cell, expressing a chimeric antigen receptor (CAR) and interleukin-12 for use in a method of treating a disease or disorder, preferably glioblastoma, wherein the multifunctional cell comprises one or two exogenous mRNA molecules encoding the CAR and interleukin-12 or each encoding the CAR and interleukin-12, wherein the disease or disorder is characterized by expression of an antigen that is bound by the CAR and wherein the cell does not contain any exogenous DNA molecules encoding the CAR and/or interleukin-12.

In a preferred embodiment, the disclosure provides a multifunctional cell, preferably a cytotoxic cell, produced according to a method according to the disclosure for use in a method of treating a disease or disorder, preferably cancer, more preferably glioblastoma.

The present disclosure is also concerned with methods of treatment or prevention of a disease or disorder, preferably cancer. The methods of treatment preferably comprise a step of administering to a subject in need thereof multifunctional cells according to the disclosure, preferably in a therapeutically effective amount. In some embodiments, the multifunctional cells are administered locally to the tumor, preferably intratumoral. In an embodiment, the multifunctional cells are not administered intratumorally.

### Methods of Production

An aspect of the disclosure is a method for producing a multifunctional cell expressing an immune receptor, such as a chimeric antigen receptor (CAR), and two or more cytokines. The herein disclosed methods are capable of producing any of the herein disclosed cells, in particular cytotoxic cells such as cytotoxic T cells.

Accordingly, the method to produce a multifunctional cell comprises the step of transfecting a mononuclear cell with one or more exogenous RNA molecules encoding the immune receptor and the two or more cytokines. These one or more RNA molecules can be any RNA molecule herein described, in particular a modified RNA.

In some embodiments, the one or more RNA molecules can be one or more linear or circular mRNA or one or more self-amplifying RNA. In case the immune receptor, such as a CAR, and the two or more cytokines are encoded by a single RNA molecule, the RNA molecule can be either an linear or circular mRNA or a self-amplifying RNA, preferably a linear mRNA. In a preferred embodiment, wherein a CAR and the two or more cytokines are encoded by different RNA molecules each of these molecules can be an linear or circular mRNA or a self-amplifying RNA, preferably a linear mRNA. In another preferred embodiment, wherein the CAR and the two or more cytokines are encoded by different RNA molecules, preferably wherein the CAR is encoded by a single RNA molecule and the two or more cytokines by a different RNA molecule or each cytokine by a separate RNA molecule, the RNA molecules can be a mixture of linear or circular mNRAs and self-amplifying RNAs, for example, the CAR is encoded by a linear mRNA and each cytokine by a self-amplifying RNA.

The method of transfection is not particularly limited, but can be any suitable method known to the skilled person. In a preferred embodiment, the transfection of the one or more exogenous RNA molecules is by a method selected from the group consisting of electroporation, lipid-mediated transfection, calcium phosphate transfection, targeted liposomes, polymer-mediated transfection, particle mediated delivery, microbubble-assisted focused ultrasound (FUS) and others, preferably from the group consisting of electroporation, lipid-mediated transfection and calcium phosphate transfection. Particularly preferred is a transfection by electroporation. In a preferred embodiment, the method of transfection is a lipid-mediated transfection and the RNA molecule is complexed with a lipid particle as described herein.

### RNA Particles

In a preferred embodiment, whether the one or more RNA molecules are transfected, or whether the one or more RNA molecules are administered to a patient, the RNA molecules are formulated as a particle, preferably a lipid particle.

In the context of the present disclosure, the term “particle” relates to a structured entity formed by molecules or molecule complexes, in particular particle forming compounds. In some embodiments, the particle contains an envelope (e.g., one or more layers or lamellas) made of one or more types of amphiphilic substances (e.g., amphiphilic lipids). In this context, the expression “amphiphilic substance” means that the substance possesses both hydrophilic and lipophilic properties. The envelope may also comprise additional substances (e.g., additional lipids) which do not have to be amphiphilic. Thus, the particle may be a monolamellar or multilamellar structure, wherein the substances constituting the one or more layers or lamellas comprise one or more types of amphiphilic substances (in particular selected from the group consisting of amphiphilic lipids) optionally in combination with additional substances (e.g., additional lipids) which do not have to be amphiphilic. In some embodiments, the term “particle” relates to a micro- or nano-sized structure, such as a micro- or nano-sized compact structure. According to the present disclosure, the term “particle” preferably includes nanoparticles.

An “RNA particle” or simply “particle” can be used to transfect RNA to a target cell, preferably T cell. An RNA particle may be formed from lipids comprising at least one cationic or cationically ionizable lipid or lipid-like material. Without intending to be bound by any theory, it is believed that the cationic or cationically ionizable lipid or lipid-like material combines together with the RNA to form aggregates, and this aggregation results in colloidally stable particles.

In one embodiment, the particles formed in the composition of the present disclosure are nanoparticles. In that embodiment, the composition according to the present disclosure comprises RNA in the form of nanoparticles. Nanoparticles can be obtained by various protocols and with various complexing compounds. Lipids, polymers, oligomers, or amphiphiles are typical constituents of nanoparticulates.

As used herein, the term “nanoparticle” refers to any particle having a diameter making the particle suitable for systemic, in particular parenteral, administration, of, in particular, nucleic acids, typically a diameter of 1000 nanometers (nm) or less. In one embodiment, the nanoparticles have an average diameter in the range of from about 50 nm to about 1000 nm, preferably from about 50 nm to about 400 nm, preferably about 100 nm to about 300 nm such as about 150 nm to about 200 nm. In one embodiment, the nanoparticles have a diameter in the range of about 200 to about 700 nm, about 200 to about 600 nm, preferably about 250 to about 550 nm, in particular about 300 to about 500 nm or about 200 to about 400 nm. In one embodiment, the average diameter is between about 50 to 150 nm, preferably, about 60 to 120 nm. In one embodiment, the average diameter is less than 50 nm.

In some embodiments, the particles are nanoparticles, in which:
(i) the number of positive charges in the nanoparticles does not exceed the number of negative charges in the nanoparticles and/or
(ii) the nanoparticles have a net negative charge and/or
(iii) the charge ratio of positive charges to negative charges in the nanoparticles is 1.4:1 or less and/or
(iv) the zeta potential of the nanoparticles is 0 or less.

In some embodiments, the charge ratio of positive charges to negative charges in the nanoparticles is between 1:1 and 1:8, preferably between 1:1 and 1:4.

RNA particles described herein preferably include lipid nanoparticle (LNP)-based and lipoplex (LPX)-based formulations.

In general, a lipoplex (LPX) is obtainable from mixing two aqueous phases, namely a phase comprising RNA and a phase comprising a dispersion of lipids. In some embodiments, the lipid phase comprises liposomes.

In some embodiments, liposomes are self-closed unilamellar or multilamellar vesicular particles wherein the lamellae comprise lipid bilayers and the encapsulated lumen comprises an aqueous phase. A prerequisite for using liposomes for nanoparticle formation is that the lipids in the mixture as required are able to form lamellar (bilayer) phases in the applied aqueous environment.

In some embodiments, liposomes comprise unilamellar or multilamellar phospholipid bilayers enclosing an aqueous core (also referred to herein as an aqueous lumen). They may be prepared from materials possessing polar head (hydrophilic) groups and nonpolar tail (hydrophobic) groups. In some embodiments, cationic lipids employed in formulating liposomes designed for the delivery of nucleic acids are amphiphilic in nature and consist of a positively charged (cationic) amine head group linked to a hydrocarbon chain or cholesterol derivative via glycerol.

In some embodiments, lipoplexes are multilamellar liposome-based formulations that form upon electrostatic interaction of cationic liposomes with RNAs. In some embodiments, formed lipoplexes possess distinct internal arrangements of molecules that arise due to the transformation from liposomal structure into compact RNA-lipoplexes. In some embodiments, these formulations are characterized by their poor encapsulation of the RNA and incomplete entrapment of the RNA.

In some embodiments, an LPX particle comprises an amphiphilic lipid, in particular cationic or cationically ionizable amphiphilic lipid, and RNA (especially mRNA) as described herein. In some embodiments, electrostatic interactions between positively charged liposomes (made from one or more amphiphilic lipids, in particular cationic or cationically ionizable amphiphilic lipids) and negatively charged nucleic acid (especially mRNA) results in complexation and spontaneous formation of nucleic acid lipoplex particles. Positively charged liposomes may be generally synthesized using a cationic or cationically ionizable amphiphilic lipid, such as DOTMA and/or DODMA, and additional lipids, such as DOPE. In some embodiments, an RNA (especially mRNA) lipoplex particle is a nanoparticle.

In general, a lipid nanoparticle (LNP) is obtainable from direct mixing of RNA in an aqueous phase with lipids in a phase comprising an organic solvent, such as ethanol. In that case, lipids or lipid mixtures can be used for particle formation, which do not form lamellar (bilayer) phases in water.

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually insoluble or poorly soluble in water, but soluble in many organic solvents. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment.

Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

As used herein, the term “hydrophobic” refers to any a molecule, moiety or group which is substantially immiscible or insoluble in aqueous solution. The term hydrophobic group includes hydrocarbons having at least 6 carbon atoms. The hydrophobic group can have functional groups (e.g., ether, ester, halide, etc.) and atoms other than carbon and hydrogen as long as the group satisfies the condition of being substantially immiscible or insoluble in aqueous solution.

The term “hydrocarbon” includes alkyl, alkenyl, or alkynyl as defined herein. It should be appreciated that one or more of the hydrogens in alkyl, alkenyl, or alkynyl may be substituted with other atoms, e.g., halogen, oxygen or sulfur. Unless stated otherwise, hydrocarbon groups can also include a cyclic (alkyl, alkenyl or alkynyl) group or an aryl group, provided that the overall polarity of the hydrocarbon remains relatively nonpolar.

The term “alkyl” refers to a saturated linear or branched monovalent hydrocarbon moiety which may have six to thirty, typically six to twenty, often six to eighteen carbon atoms. Exemplary nonpolar alkyl groups include, but are not limited to, hexyl, decyl, dodecyl, tetradecyl, hexadecyl, octadecyl, and the like.

The term “alkenyl” refers to a linear or branched monovalent hydrocarbon moiety having at least one carbon carbon double bond in which the total carbon atoms may be six to thirty, typically six to twenty often six to eighteen.

The term “alkynyl” refers to a linear or branched monovalent hydrocarbon moiety having at least one carbon carbon triple bond in which the total carbon atoms may be six to thirty, typically six to twenty, often six to eighteen. Alkynyl groups can optionally have one or more carbon carbon double bonds.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances, in particular amphiphilic substances, that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term includes molecules, which comprise hydrophilic and hydrophobic moieties with different structural organization, which may or may not be similar to that of lipids. Examples of lipid-like compounds capable of spontaneous integration into cell membranes include functional lipid constructs such as synthetic function-spacer-lipid constructs (FSL), synthetic function-spacer-sterol constructs (FSS) as well as artificial amphipathic molecules. Lipids are generally cylindrical. The area occupied by the two alkyl chains is similar to the area occupied by the polar head group. Lipids have low solubility as monomers and tend to aggregate into planar bilayers that are water insoluble. Traditional surfactant monomers are generally cone shaped. The hydrophilic head groups tend to occupy more molecular space than the linear alkyl chains. In some embodiments, surfactants tend to aggregate into spherical or elliptoid micelles that are water soluble. While lipids also have the same general structure as surfactants—a polar hydrophilic head group and a nonpolar hydrophobic tail—lipids differ from surfactants in the shape of the monomers, in the type of aggregates formed in solution, and in the concentration range required for aggregation. As used herein, the term “lipid” is to be construed to cover both lipids and lipid-like materials unless otherwise indicated herein or clearly contradicted by context.

Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as steroids, i.e., sterol-containing metabolites such as cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

Preferably, the lipid particle according to the disclosure comprises the one or more RNA molecule, one or more lipids and optionally further substances. In a preferred embodiment the lipid particle comprises a cationic lipid. As used herein, a “cationic lipid” refers to a lipid or lipid-like material having a net positive charge. Cationic lipids bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge.

In one embodiment, the lipid particle according to the disclosure comprises RNA encapsulated in a vesicle. Such formulation is a particular particle according to the disclosure. A vesicle is a lipid bilayer rolled up into a spherical shell, enclosing a small space and separating that space from the space outside the vesicle. Typically, the space inside the vesicle is an aqueous space, i.e. comprises water. Typically, the space outside the vesicle is an aqueous space, i.e. comprises water. The lipid bilayer is formed by one or more lipids (vesicle-forming lipids). The membrane enclosing the vesicle is a lamellar phase, similar to that of the plasma membrane. The vesicle according to the present disclosure may be a multilamellar vesicle, a unilamellar vesicle, or a mixture thereof. When encapsulated in a vesicle, the RNA is typically separated from any external medium. Thus, it is present in protected form, functionally equivalent to the protected form in, e.g., a natural alphavirus. Suitable vesicles are particles, particularly nanoparticles, as described herein.

For example, RNA may be encapsulated in a liposome. In that embodiment, Encapsulation within a liposome will typically protect RNA from RNase digestion. It is possible that the liposomes include some external RNA (e.g. on their surface), but at least half of the RNA (and ideally all of it) is encapsulated within the core of the liposome.

Liposomes are microscopic lipidic vesicles often having one or more bilayers of a vesicle-forming lipid, such as a phospholipid, and are capable of encapsulating a drug, e.g. RNA. Different types of liposomes may be employed in the context of the present disclosure, including, without being limited thereto, multilamellar vesicles (MLV), small unilamellar vesicles (SUV), large unilamellar vesicles (LUV), sterically stabilized liposomes (SSL), multivesicular vesicles (MV), and large multivesicular vesicles (LMV) as well as other bilayered forms known in the art. The size and lamellarity of the liposome will depend on the manner of preparation. There are several other forms of supramolecular organization in which lipids may be present in an aqueous medium, comprising lamellar phases, hexagonal and inverse hexagonal phases, cubic phases, micelles, reverse micelles composed of monolayers. These phases may also be obtained in the combination with DNA or RNA, and the interaction with RNA and DNA may substantially affect the phase state. Such phases may be present in nanoparticulate RNA formulations of the present disclosure.

Liposomes may be formed using standard methods known to the skilled person. Respective methods include the reverse evaporation method, the ethanol injection method, the dehydration-rehydration method, sonication or other suitable methods. Following liposome formation, the liposomes can be sized to obtain a population of liposomes having a substantially homogeneous size range.

In a preferred embodiment of the present disclosure, the RNA is present in a liposome which includes at least one cationic lipid. Respective liposomes can be formed from a single lipid or from a mixture of lipids, provided that at least one cationic lipid is used. Preferred cationic lipids have a nitrogen atom which is capable of being protonated; preferably, such cationic lipids are lipids with a tertiary amine group. A particularly suitable lipid with a tertiary amine group is 1,2-dilinoleyloxy-N,N-dimethyl-3-aminopropane (DLinDMA). In one embodiment, the RNA according to the present disclosure is present in a liposome formulation as described in WO 2012/006378 A1: a liposome having a lipid bilayer encapsulating an aqueous core including RNA, wherein the lipid bilayer comprises a lipid with a pKa in the range of 5.0 to 7.6, which preferably has a tertiary amine group. Preferred cationic lipids with a tertiary amine group include DLinDMA (pKa 5.8) and are generally described in WO 2012/031046 A2. According to WO 2012/031046 A2, liposomes comprising a respective compound are particularly suitable for encapsulation of RNA and thus liposomal delivery of RNA. In one embodiment, the RNA according to the present disclosure is present in a liposome formulation, wherein the liposome includes at least one cationic lipid whose head group includes at least one nitrogen atom (N) which is capable of being protonated, wherein the liposome and the RNA have a N:P ratio of between 1:1 and 20:1. According to the present disclosure, “N:P ratio” refers to the molar ratio of nitrogen atoms (N) in the cationic lipid to phosphate atoms (P) in the RNA comprised in a lipid containing particle (e.g. liposome), as described in WO 2013/006825 A1. The N:P ratio of between 1:1 and 20:1 is implicated in the net charge of the liposome and in efficiency of delivery of RNA to a vertebrate cell.

In one embodiment, the RNA according to the present disclosure is present in a liposome formulation that comprises at least one lipid which includes a polyethylene glycol (PEG) moiety, wherein RNA is encapsulated within a PEGylated liposome such that the PEG moiety is present on the liposome's exterior, as described in WO 2012/031043 A1 and WO 2013/033563 A1.

In one embodiment, the RNA according to the present disclosure is present in a liposome formulation, wherein the liposome has a diameter in the range of 60-180 nm, as described in WO 2012/030901 A1. In one embodiment, the RNA according to the present disclosure is present in a liposome formulation, wherein the RNA-containing liposomes have a net charge close to zero or negative, as disclosed in WO 2013/143555 A1.

In other embodiments, the RNA according to the present disclosure is present in the form of an emulsion. Emulsions have been previously described to be used for delivery of nucleic acid molecules, such as RNA molecules, to cells. Preferred herein are oil-in-water emulsions. The respective emulsion particles comprise an oil core and a cationic lipid. More preferred are cationic oil-in-water emulsions in which the RNA according to the present disclosure is complexed to the emulsion particles. The emulsion particles comprise an oil core and a cationic lipid. The cationic lipid can interact with the negatively charged RNA, thereby anchoring the RNA to the emulsion particles. In an oil-in-water emulsion, emulsion particles are dispersed in an aqueous continuous phase. For example, the average diameter of the emulsion particles may typically be from about 80 nm to 180 nm. In one embodiment, the composition of the present disclosure is a cationic oil-in-water emulsion, wherein the emulsion particles comprise an oil core and a cationic lipid, as described in WO 2012/006380 A2. The RNA according to the present disclosure may be present in the form of an emulsion comprising a cationic lipid wherein the N:P ratio of the emulsion is at least 4:1, as described in WO 2013/006834 A1. The RNA according to the present disclosure may be present in the form of a cationic lipid emulsion, as described in WO 2013/006837 A1. In particular, the composition may comprise RNA complexed with a particle of a cationic oil-in-water emulsion, wherein the ratio of oil/lipid is at least about 8:1 (mole:mole).

In other embodiments, the lipid particle according to the disclosure comprises RNA in the format of a lipoplex. The term, “lipoplex” or “RNA lipoplex” refers to a complex of lipids and nucleic acids such as RNA. Lipoplexes can be formed of cationic (positively charged) liposomes and the anionic (negatively charged) nucleic acid. The cationic liposomes can also include a neutral “helper” lipid. In the simplest case, the lipoplexes form spontaneously by mixing the nucleic acid with the liposomes with a certain mixing protocol, however various other protocols may be applied. It is understood that electrostatic interactions between positively charged liposomes and negatively charged nucleic acid are the driving force for the lipoplex formation (WO 2013/143555 A1). In one embodiment of the present disclosure, the net charge of the RNA lipoplex particles is close to zero or negative. It is known that electro-neutral or negatively charged lipoplexes of RNA and liposomes lead to substantial RNA expression in spleen dendritic cells (DCs) after systemic administration and are not associated with the elevated toxicity that has been reported for positively charged liposomes and lipoplexes (cf. WO 2013/143555 A1). Therefore, in one embodiment of the present disclosure, the composition according to the disclosure comprises RNA in the format of nanoparticles, preferably lipoplex nanoparticles, in which (i) the number of positive charges in the nanoparticles does not exceed the number of negative charges in the nanoparticles and/or (ii) the nanoparticles have a neutral or net negative charge and/or (iii) the charge ratio of positive charges to negative charges in the nanoparticles is 1.4:1 or less and/or (iv) the zeta potential of the nanoparticles is 0 or less. As described in WO 2013/143555 A1, zeta potential is a scientific term for electrokinetic potential in colloidal systems. In the present disclosure, (a) the zeta potential and (b) the charge ratio of the cationic lipid to the RNA in the nanoparticles can both be calculated as disclosed in WO 2013/143555 A1. In summary, particles which are nanoparticulate lipoplex formulations with a defined particle size, wherein the net charge of the particles is close to zero or negative, as disclosed in WO 2013/143555 A1, are preferred particles in the context of the present disclosure.

In some embodiments, the lipid particle comprises nanoparticles formed by the RNA molecule and at least one lipid, which are lipid nanoparticles (LNPs). The LNP may comprise any lipid capable of forming a particle to which the nucleic acid molecules are attached, or in which the nucleic acid molecules are encapsulated.

In one embodiment, the LNP comprises one or more cationic lipids, and one or more stabilizing lipids. Stabilizing lipids include neutral lipids and pegylated lipids.

In one embodiment, the LNP comprises a cationic lipid, a neutral lipid, a steroid, a polymer conjugated lipid; and the RNA, encapsulated within or associated with the lipid nanoparticle. In one embodiment, the LNP comprises from 40 to 55 mol percent, from 40 to 50 mol percent, from 41 to 49 mol percent, from 41 to 48 mol percent, from 42 to 48 mol percent, from 43 to 48 mol percent, from 44 to 48 mol percent, from 45 to 48 mol percent, from 46 to 48 mol percent, from 47 to 48 mol percent, or from 47.2 to 47.8 mol percent of the cationic lipid. In one embodiment, the LNP comprises about 47.0, 47.1, 47.2, 47.3, 47.4, 47.5, 47.6, 47.7, 47.8, 47.9 or 48.0 mol percent of the cationic lipid.

In one embodiment, the neutral lipid is present in a concentration ranging from 5 to 15 mol percent, from 7 to 13 mol percent, or from 9 to 11 mol percent. In one embodiment, the neutral lipid is present in a concentration of about 9.5, 10 or 10.5 mol percent.

In one embodiment, the steroid is present in a concentration ranging from 30 to 50 mol percent, from 35 to 45 mol percent or from 38 to 43 mol percent. In one embodiment, the steroid is present in a concentration of about 40, 41, 42, 43, 44, 45 or 46 mol percent. In one embodiment, the LNP comprises from 1 to 10 mol percent, from 1 to 5 mol percent, or from 1 to 2.5 mol percent of the polymer conjugated lipid.

In one embodiment, the LNP comprises from 40 to 50 mol percent a cationic lipid; from 5 to 15 mol percent of a neutral lipid; from 35 to 45 mol percent of a steroid; from 1 to 10 mol percent of a polymer conjugated lipid; and the RNA, encapsulated within or associated with the lipid nanoparticle.

In one embodiment, the mol percent is determined based on total mol of lipid present in the lipid nanoparticle.

In one embodiment, the neutral lipid is selected from the group consisting of DSPC, DPPC, DMPC, DOPC, POPC, DOPE, DOPG, DPPG, POPE, DPPE, DMPE, DSPE, and SM. In one embodiment, the neutral lipid is selected from the group consisting of DSPC, DPPC, DMPC, DOPC, POPC, DOPE and SM. In one embodiment, the neutral lipid is DSPC.

In one embodiment, the steroid is cholesterol.

In one embodiment, the polymer conjugated lipid is a pegylated lipid. In one embodiment, the pegylated lipid has the following structure.

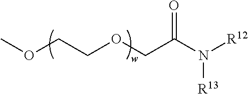

or a pharmaceutically acceptable salt, tautomer or stereoisomer thereof, wherein:
R¹² and R¹³ are each independently a straight or branched, saturated or unsaturated alkyl chain containing from 10 to 30 carbon atoms, wherein the alkyl chain is optionally interrupted by one or more ester bonds; and w has a mean value ranging from 30 to 60. In one embodiment, R¹² and R¹³ are each independently straight, saturated alkyl chains containing from 12 to 16 carbon atoms. In one embodiment, w has a mean value ranging from 40 to 55. In one embodiment, the average w is about 45. In one embodiment, R¹² and R¹³ are each independently a straight, saturated alkyl chain containing about 14 carbon atoms, and w has a mean value of about 45.

In one embodiment, the pegylated lipid is DMG-PEG 2000, e.g., having the following structure:

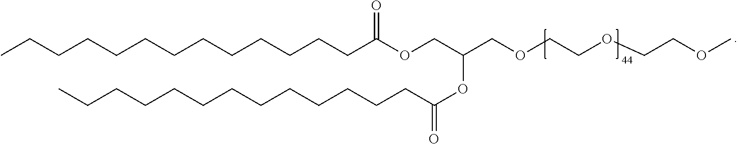

In some embodiments, the cationic lipid component of the LNPs has the structure of Formula (III):

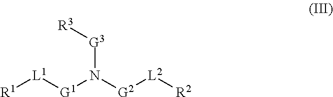

or a pharmaceutically acceptable salt, tautomer, prodrug or stereoisomer thereof, wherein:
one of L¹ or L² is —O(C═O)—, —(C═O)O—, —C(═O)—, —O—, —S(O)ₓ—, —S—S—, —C(═O)S—, SC(═O)—, —NR^{a}C(═O)—, —C(═O) NR^{a}—, NR^{a} C(═O) NR^{a}—, —OC(═O) NR^{a}— or —NR^{a} C(═O)O—, and the other of L¹ or L² is —O(C═O)—, —(C═O)O—, —C(═O)—, —O—, —S(O)ₓ—, —S—S—, —C(═O)S—, SC(═O)—, —NR^{a} C(═O)—, —C(═O) NR^{a}—, NR^{a} C(═O) NR^{a}—, —OC(═O) NR^{a}— or —NR^{a} C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O) OR⁴, —OC(═O) R⁴ or —NR⁵C(═O) R⁴;
R⁴ is C₁-C₁₂ alkyl;
R⁵ is H or C₁-C₆ alkyl; and
x is 0, 1 or 2.

In some of the foregoing embodiments of Formula (III), the lipid has one of the following structures (IIIA) or (IIIB):

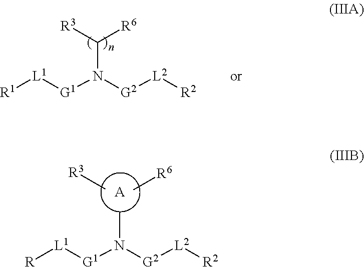

wherein:
A is a 3 to 8-membered cycloalkyl or cycloalkylene ring;
R⁶ is, at each occurrence, independently H, OH or C₁-C₂₄ alkyl;
n is an integer ranging from 1 to 15.

In some of the foregoing embodiments of Formula (III), the lipid has structure (IIIA), and in other embodiments, the lipid has structure (IIIB).

In other embodiments of Formula (III), the lipid has one of the following structures (IIIC) or (IIID):

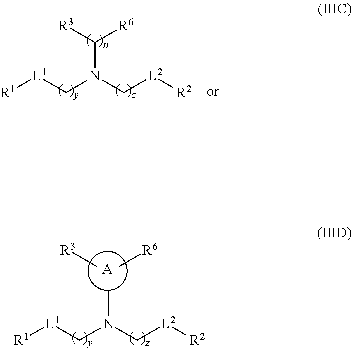

wherein y and z are each independently integers ranging from 1 to 12.

In any of the foregoing embodiments of Formula (III), one of L¹ or L² is —O(C═O)—. For example, in some embodiments each of L¹ and L² are —O(C═O)—. In some different embodiments of any of the foregoing, L¹ and L² are each independently —(C═O)O— or —O(C═O)—. For example, in some embodiments each of L¹ and L² is —(C═O)O—.

In some different embodiments of Formula (III), the lipid has one of the following structures (IIIE) or (IIIF):

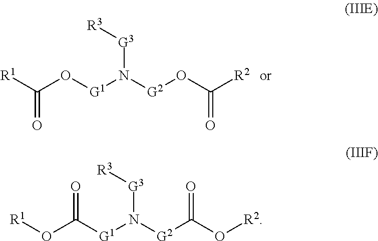

In some of the foregoing embodiments of Formula (III), the lipid has one of the following structures (IIIG), (IIIH), (IIII), or (IIIJ):

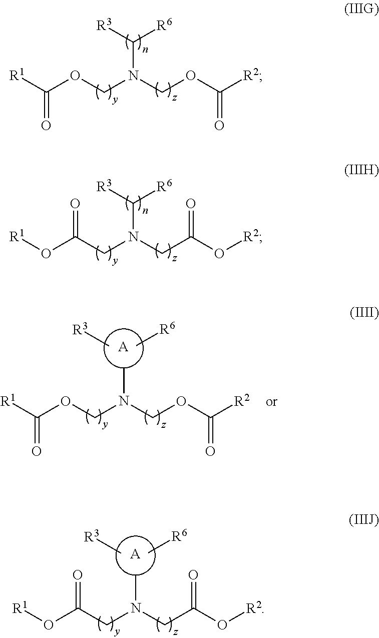

In some of the foregoing embodiments of Formula (III), n is an integer ranging from 2 to 12, for example from 2 to 8 or from 2 to 4. For example, in some embodiments, n is 3, 4, 5 or 6. In some embodiments, n is 3. In some embodiments, n is 4. In some embodiments, n is 5. In some embodiments, n is 6.

In some other of the foregoing embodiments of Formula (III), y and z are each independently an integer ranging from 2 to 10. For example, in some embodiments, y and z are each independently an integer ranging from 4 to 9 or from 4 to 6.

In some of the foregoing embodiments of Formula (III), R⁶ is H. In other of the foregoing embodiments, R⁶ is C₁-C₂₄ alkyl. In other embodiments, R⁶ is OH.

In some embodiments of Formula (III), G³ is unsubstituted. In other embodiments, G³ is substituted. In various different embodiments, G³ is linear C₁-C₂₄ alkylene or linear C₁-C₂₄ alkenylene.

In some other foregoing embodiments of Formula (III), R¹ or R², or both, is C₆-C₂₄ alkenyl. For example, in some embodiments, R¹ and R² each, independently have the following structure:

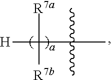

wherein:
R^{7a} and R^{7b} are, at each occurrence, independently H or C₁-C₁₂ alkyl; and
a is an integer from 2 to 12,
wherein R^{7a}, R^{7b} and a are each selected such that R¹ and R² each independently comprise from 6 to 20 carbon atoms. For example, in some embodiments a is an integer ranging from 5 to 9 or from 8 to 12.

In some of the foregoing embodiments of Formula (III), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (III), R¹ or R², or both, has one of the following structures:

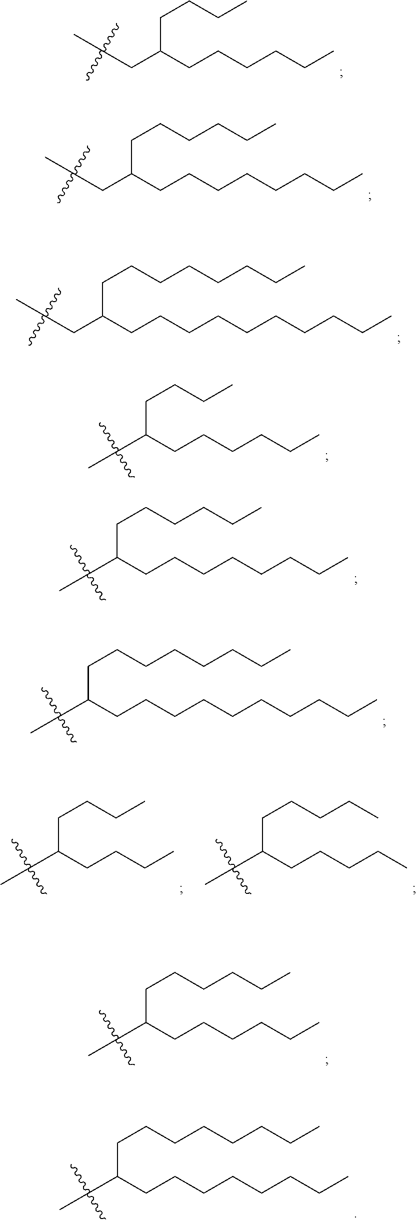

In some of the foregoing embodiments of Formula (III), R³ is OH, CN, —C(═O) OR⁴, —OC(═O) R⁴ or —NHC(═O) R⁴. In some embodiments, R⁴ is methyl or ethyl.

In various different embodiments, the cationic lipid of Formula (III) has one of the structures set forth in the table below.

### Representative Compounds of Formula (III):

| No. | Structure |
|---|---|
| III-1 | 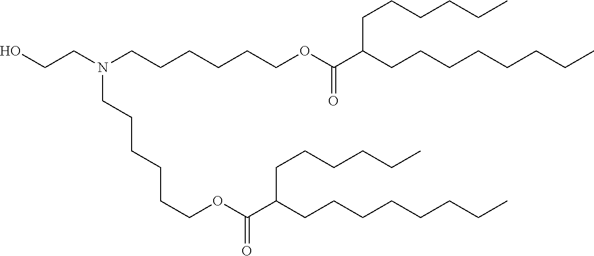 [SMILES: CCCCCCCCC(CCCCCC)C(=O)OCCCCCCN(CCO)CCCCCCOC(=O)C(CCCCCC)CCCCCCCC] |
| III-2 | 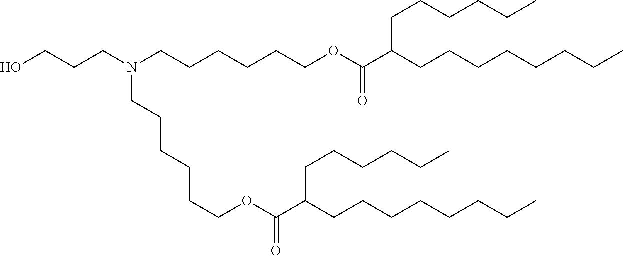 [SMILES: CCCCCCCCC(CCCCCC)C(=O)OCCCCCCN(CCCO)CCCCCCOC(=O)C(CCCCCC)CCCCCCCC] |
| III-3 | 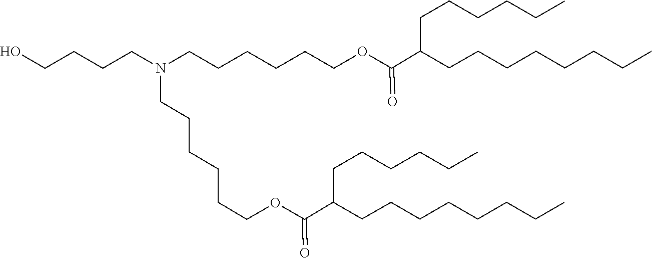 [SMILES: CCCCCCCCC(CCCCCC)C(=O)OCCCCCCN(CCCCO)CCCCCCOC(=O)C(CCCCCC)CCCCCCCC] |
| III-4 | 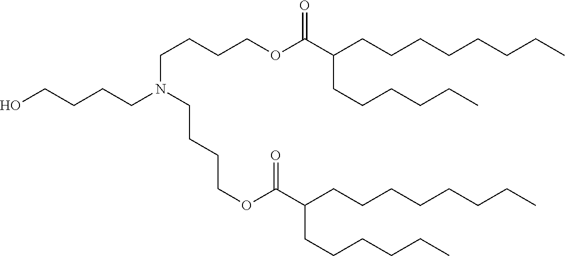 [SMILES: CCCCCCCCC(CCCCCC)C(=O)OCCCCN(CCCCO)CCCCOC(=O)C(CCCCCC)CCCCCCCC] |
| III-5 | 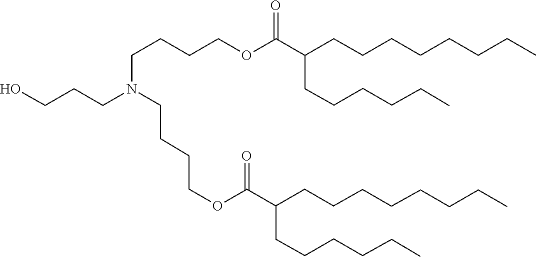 [SMILES: CCCCCCCCC(CCCCCC)C(=O)OCCCCN(CCCO)CCCCOC(=O)C(CCCCCC)CCCCCCCC] |
| III-6 | 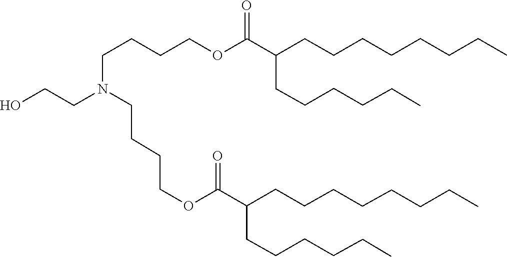 [SMILES: CCCCCCCCC(CCCCCC)C(=O)OCCCCN(CCO)CCCCOC(=O)C(CCCCCC)CCCCCCCC] |
| III-7 | 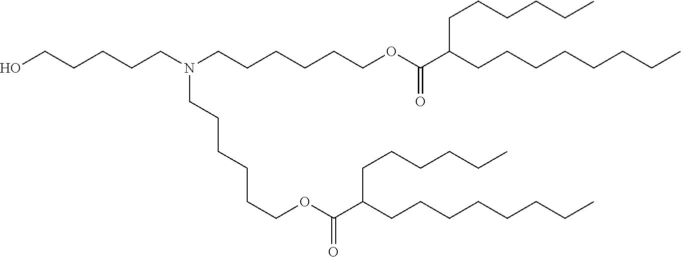 [SMILES: CCCCCCCCC(CCCCCC)C(=O)OCCCCCCN(CCCCCO)CCCCCCOC(=O)C(CCCCCC)CCCCCCCC] |
| III-8 | 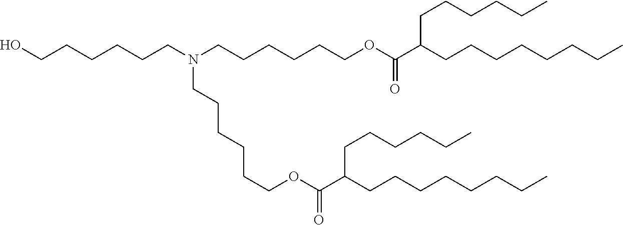 [SMILES: CCCCCCCCC(CCCCCC)C(=O)OCCCCCCN(CCCCCCO)CCCCCCOC(=O)C(CCCCCC)CCCCCCCC] |
| III-9 | 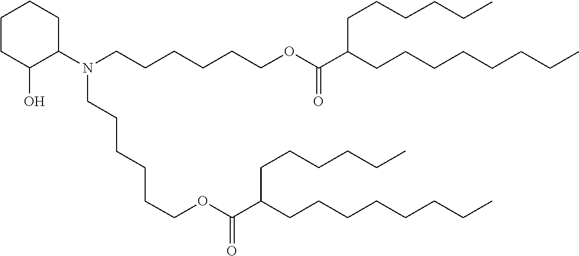 [SMILES: CCCCCCCCC(CCCCCC)C(=O)OCCCCCCN(CCCCCCOC(=O)C(CCCCCC)CCCCCCCC)C1CCCCC1O] |
| III-10 | 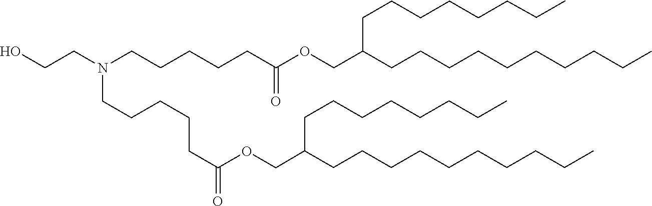 [SMILES: CCCCCCCCCCC(CCCCCCCC)COC(=O)CCCCCN(CCO)CCCCCC(=O)OCC(CCCCCCCC)CCCCCCCCCC] |
| III-11 | 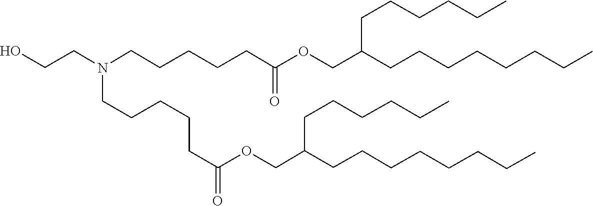 [SMILES: CCCCCCCCC(CCCCCC)COC(=O)CCCCCN(CCO)CCCCCC(=O)OCC(CCCCCC)CCCCCCCC] |
| III-12 | 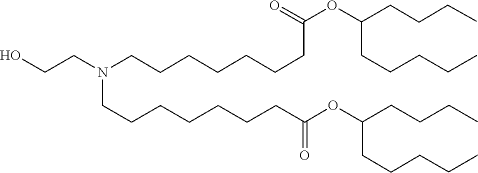 [SMILES: CCCCCC(CCCC)OC(=O)CCCCCCCN(CCO)CCCCCCCC(=O)OC(CCCC)CCCCC] |
| III-13 | 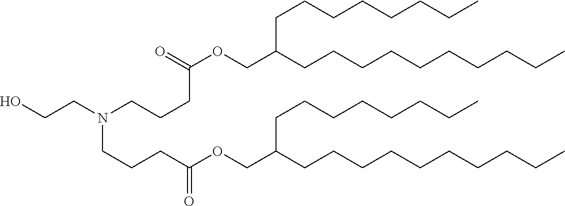 [SMILES: CCCCCCCCCCC(CCCCCCCC)COC(=O)CCCN(CCO)CCCC(=O)OCC(CCCCCCCC)CCCCCCCCCC] |
| III-14 | 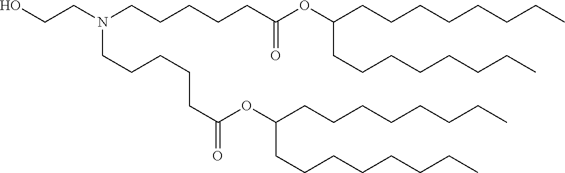 [SMILES: CCCCCCCCC(CCCCCCCC)OC(=O)CCCCCN(CCO)CCCCCC(=O)OC(CCCCCCCC)CCCCCCCC] |
| III-15 | 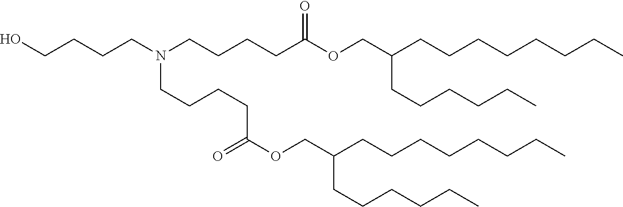 [SMILES: CCCCCCCCC(CCCCCC)COC(=O)CCCCN(CCCCO)CCCCC(=O)OCC(CCCCCC)CCCCCCCC] |
| III-16 | 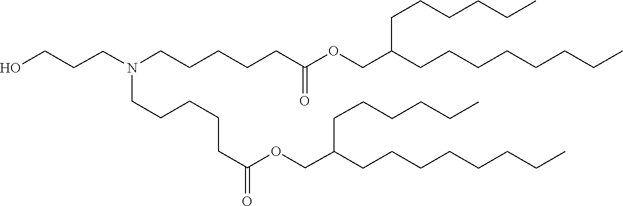 [SMILES: CCCCCCCCC(CCCCCC)COC(=O)CCCCCN(CCCO)CCCCCC(=O)OCC(CCCCCC)CCCCCCCC] |
| III-17 | 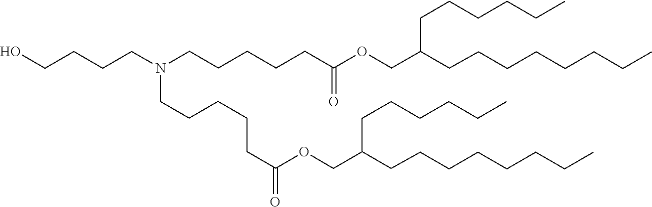 [SMILES: CCCCCCCCC(CCCCCC)COC(=O)CCCCCN(CCCCO)CCCCCC(=O)OCC(CCCCCC)CCCCCCCC] |
| III-18 | 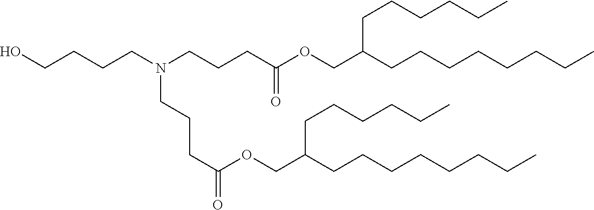 [SMILES: CCCCCCCCC(CCCCCC)COC(=O)CCCN(CCCCO)CCCC(=O)OCC(CCCCCC)CCCCCCCC] |
| III-19 | 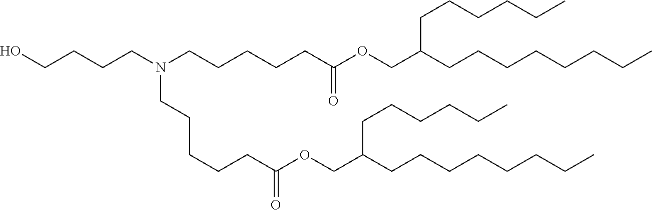 [SMILES: CCCCCCCCC(CCCCCC)COC(=O)CCCCCN(CCCCO)CCCCCC(=O)OCC(CCCCCC)CCCCCCCC] |
| III-20 | 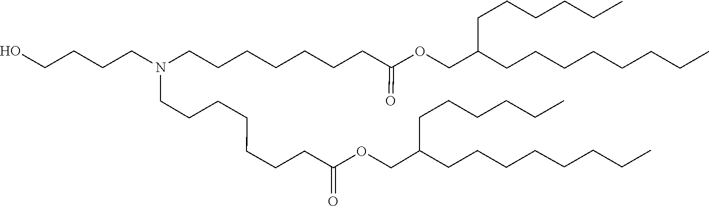 [SMILES: CCCCCCCCC(CCCCCC)COC(=O)CCCCCCCN(CCCCO)CCCCCCCC(=O)OCC(CCCCCC)CCCCCCCC] |
| III-21 | 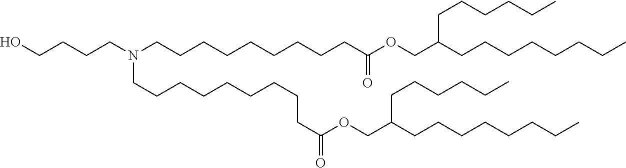 [SMILES: CCCCCCCCC(CCCCCC)COC(=O)CCCCCCCCCN(CCCCO)CCCCCCCCCC(=O)OCC(CCCCCC)CCCCCCCC] |
| III-22 | 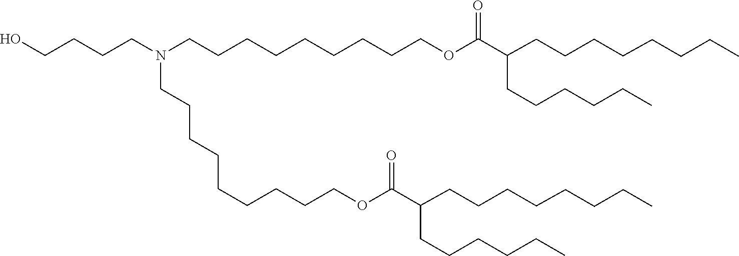 [SMILES: CCCCCCCCC(CCCCCC)C(=O)OCCCCCCCCCN(CCCCO)CCCCCCCCCOC(=O)C(CCCCCC)CCCCCCCC] |
| III-23 | 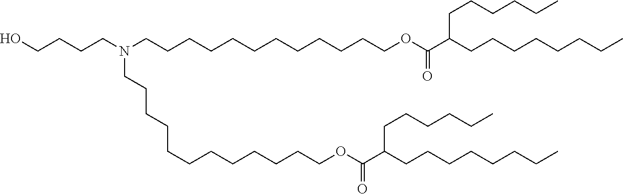 [SMILES: CCCCCCCCC(CCCCCC)C(=O)OCCCCCCCCCCCCN(CCCCO)CCCCCCCCCCCCOC(=O)C(CCCCCC)CCCCCCCC] |
| III-24 | 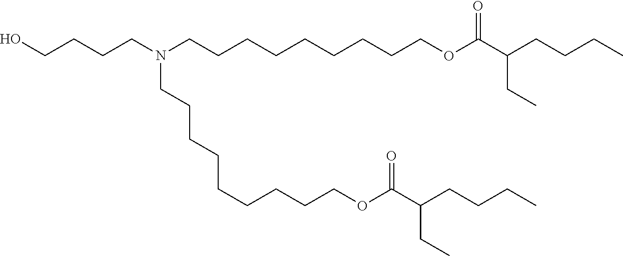 [SMILES: CCCCC(CC)C(=O)OCCCCCCCCCN(CCCCO)CCCCCCCCCOC(=O)C(CC)CCCC] |
| III-25 | 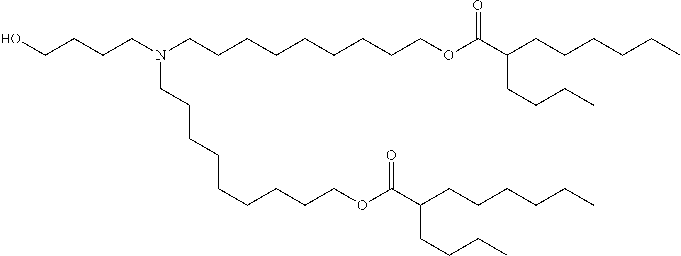 [SMILES: CCCCCCC(CCCC)C(=O)OCCCCCCCCCN(CCCCO)CCCCCCCCCOC(=O)C(CCCC)CCCCCC] |
| III-26 | 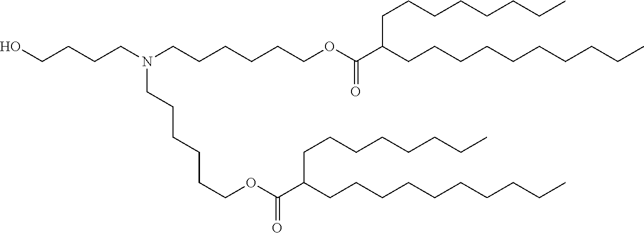 [SMILES: CCCCCCCCCCC(CCCCCCCC)C(=O)OCCCCCCN(CCCCO)CCCCCCOC(=O)C(CCCCCCCC)CCCCCCCCCC] |
| III-27 | 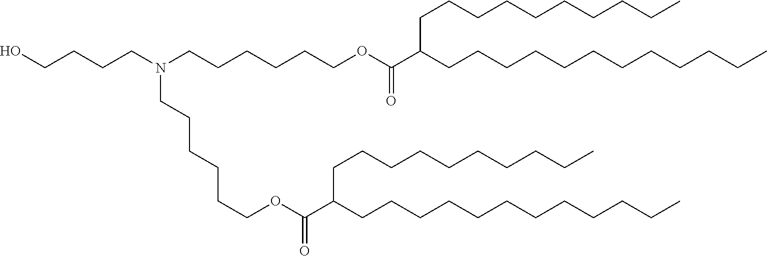 [SMILES: CCCCCCCCCCCCC(CCCCCCCCCC)C(=O)OCCCCCCN(CCCCO)CCCCCCOC(=O)C(CCCCCCCCCC)CCCCCCCCCCCC] |
| III-28 | 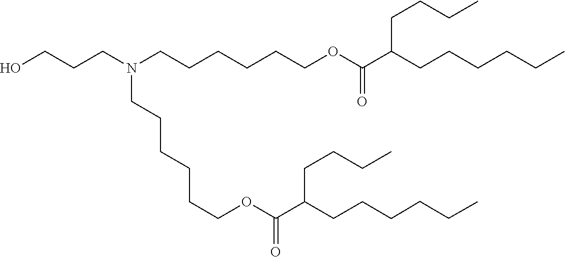 [SMILES: CCCCCCC(CCCC)C(=O)OCCCCCCN(CCCO)CCCCCCOC(=O)C(CCCC)CCCCCC] |
| III-29 | 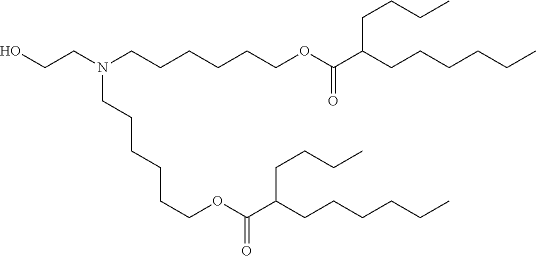 [SMILES: CCCCCCC(CCCC)C(=O)OCCCCCCN(CCO)CCCCCCOC(=O)C(CCCC)CCCCCC] |
| III-30 | 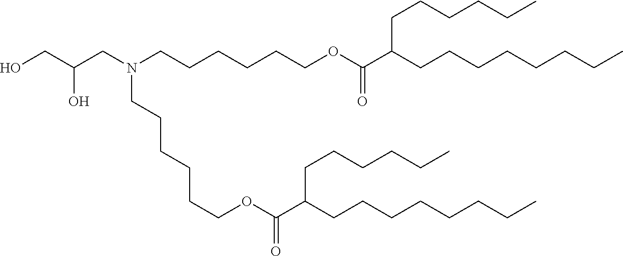 [SMILES: CCCCCCCCC(CCCCCC)C(=O)OCCCCCCN(CCCCCCOC(=O)C(CCCCCC)CCCCCCCC)CC(O)CO] |
| III-31 | 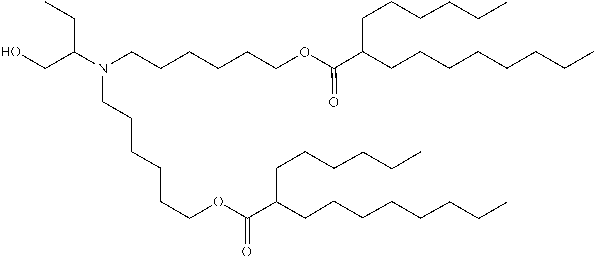 [SMILES: CCCCCCCCC(CCCCCC)C(=O)OCCCCCCN(CCCCCCOC(=O)C(CCCCCC)CCCCCCCC)C(CC)CO] |
| III-32 | 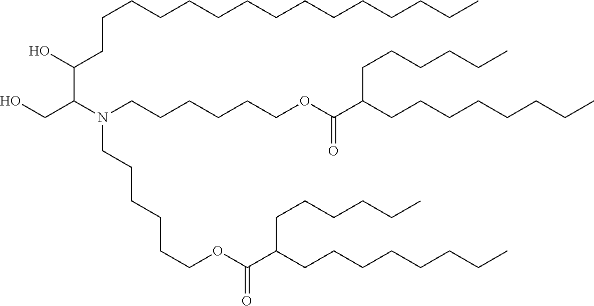 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCC] |
| III-33 | 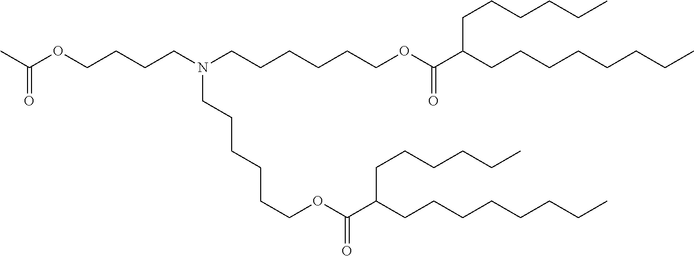 [SMILES: CCCCCCCCC(CCCCCC)C(=O)OCCCCCCN(CCCCCCOC(=O)C(CCCCCC)CCCCCCCC)CCCCOC(C)=O] |
| III-34 | 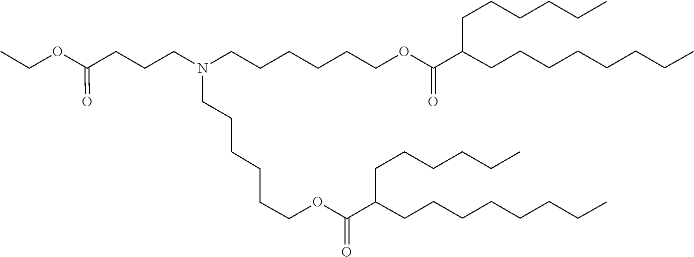 [SMILES: CCCCCCCCC(CCCCCC)C(=O)OCCCCCCN(CCCCCCOC(=O)C(CCCCCC)CCCCCCCC)CCCC(=O)OCC] |
| III-35 | 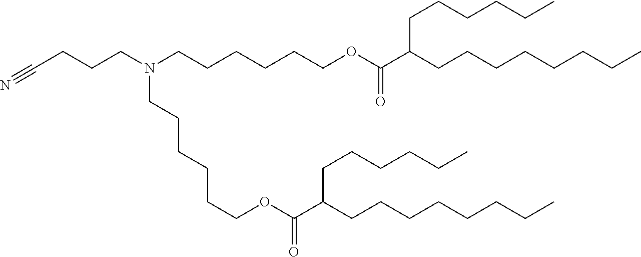 [SMILES: CCCCCCCCC(CCCCCC)C(=O)OCCCCCCN(CCCC#N)CCCCCCOC(=O)C(CCCCCC)CCCCCCCC] |
| III-36 | 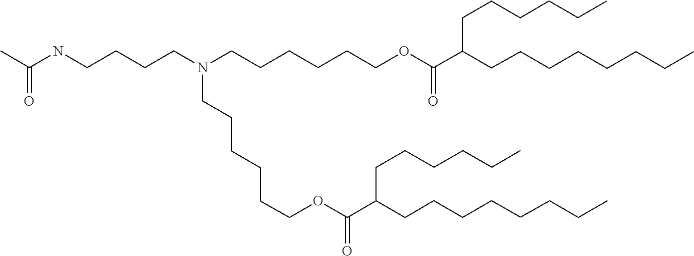 [SMILES: CCCCCCCCC(CCCCCC)C(=O)OCCCCCCN(CCCCCCOC(=O)C(CCCCCC)CCCCCCCC)CCCCNC(C)=O] |

In some embodiments, the LNP comprises a lipid of Formula (III), RNA, a neutral lipid, a steroid and a pegylated lipid. In some embodiments, the lipid of Formula (III) is compound III-3. In some embodiments, the neutral lipid is DSPC. In some embodiments, the steroid is cholesterol. In some embodiments, the pegylated lipid is ALC-0159.

In some embodiments, the cationic lipid is present in the LNP in an amount from about 40 to about 50 mole percent. In one embodiment, the neutral lipid is present in the LNP in an amount from about 5 to about 15 mole percent. In one embodiment, the steroid is present in the LNP in an amount from about 35 to about 45 mole percent. In one embodiment, the pegylated lipid is present in the LNP in an amount from about 1 to about 10 mole percent.

In some embodiments, the LNP comprises compound III-3 in an amount from about 40 to about 50 mole percent, DSPC in an amount from about 5 to about 15 mole percent, cholesterol in an amount from about 35 to about 45 mole percent, and ALC-0159 in an amount from about 1 to about 10 mole percent.

In some embodiments, the LNP comprises compound III-3 in an amount of about 47.5 mole percent, DSPC in an amount of about 10 mole percent, cholesterol in an amount of about 40.7 mole percent, and ALC-0159 in an amount of about 1.8 mole percent.

In various different embodiments, the cationic lipid has one of the structures set forth in the table below.

| No. | Structure |
|---|---|
| A | 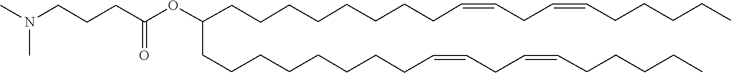 [SMILES: CCCCC/C=C\C/C=C\CCCCCCCCC(CCCCCCCC/C=C\C/C=C\CCCCC)OC(=O)CCCN(C)C] |
| B | 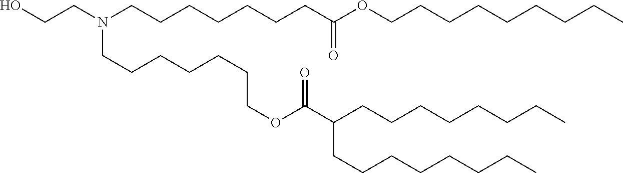 [SMILES: CCCCCCCCCOC(=O)CCCCCCCN(CCO)CCCCCCCOC(=O)C(CCCCCCCC)CCCCCCCC] |
| C | 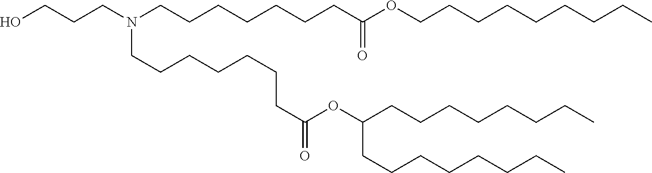 [SMILES: CCCCCCCCCOC(=O)CCCCCCCN(CCCO)CCCCCCCC(=O)OC(CCCCCCCC)CCCCCCCC] |
| D | 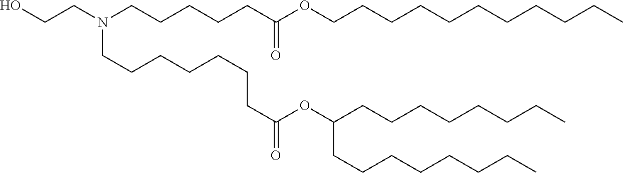 [SMILES: CCCCCCCCCCCOC(=O)CCCCCN(CCO)CCCCCCCC(=O)OC(CCCCCCCC)CCCCCCCC] |
| E | 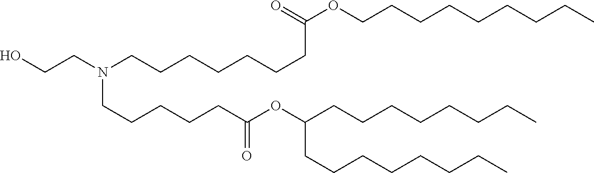 [SMILES: CCCCCCCCCOC(=O)CCCCCCCN(CCO)CCCCCC(=O)OC(CCCCCCCC)CCCCCCCC] |
| F | 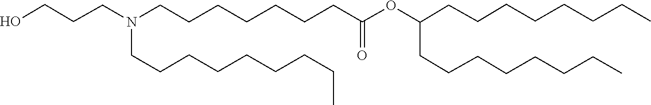 [SMILES: CCCCCCCCCN(CCCO)CCCCCCCC(=O)OC(CCCCCCCC)CCCCCCCC] |

In some embodiments, the LNP comprises a cationic lipid shown in the above table, e.g., a cationic lipid of Formula (B) or Formula (D), in particular a cationic lipid of Formula (D), RNA, a neutral lipid, a steroid and a pegylated lipid. In some embodiments, the neutral lipid is DSPC.

5 In some embodiments, the steroid is cholesterol. In some embodiments, the pegylated lipid is DMG-PEG 2000.

In one embodiment, the LNP comprises a cationic lipid that is an ionizable lipid-like material (lipidoid). In one embodiment, the cationic lipid has the following structure:

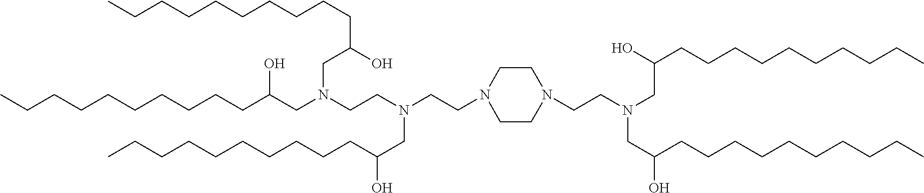 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

The N/P ratio is preferably at least about 4. In some embodiments, the N/P ratio ranges from 4 to 20, 4 to 12, 4 to 10, 4 to 8, or 5 to 7. In some embodiments, the N/P ratio is about 6.

LNPs described herein may have an average diameter that in one embodiment ranges from about 30 nm to about 200 nm, or from about 60 nm to about 120 nm.

In an embodiment, the particles can further comprise at least one helper lipid, wherein, for example, the helper lipid can be a neutral lipid. In various embodiments, the at least one cationic lipid can comprise 1,2-di-O-octadecenyl-3-trimethylammonium propane (DOTMA), 1,2-dioleyloxy-3-dimethylaminopropane (DODMA), and/or 1,2-dioleoyl-3-trimethylammonium-propane (DOTAP). In various embodiments, the at least one helper lipid can comprise 1,2-di-(9Z-octadecenoyl)-sn-glycero-3-phosphoethanolamine (DOPE), cholesterol (Chol), 1,2-dioleoyl-sn-glycero-3-phosphocholine (DOPC), and/or 1,2-distearoyl-sn-glycero-3-phosphocholine (DSPC). In various embodiments, the molar ratio of the at least one cationic lipid to the at least one helper lipid can be from 10:0 to 3:7, preferably 9:1 to 3:7, 4:1 to 1:2, 4:1 to 2:3, 7:3 to 1:1, or 2:1 to 1:1, preferably about 1:1.

In an embodiment, the particles can be lipoplexes comprising DODMA and DOPE in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2. In an embodiment, the nanoparticles can be lipoplexes comprising DODMA and Cholesterol in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2. In an embodiment, the nanoparticles can be lipoplexes comprising DODMA and DSPC in a molar ratio of 10:0 to 1:9, preferably 8:2 to 3:7, and more preferably of 7:3 to 5:5 and wherein the charge ratio of positive charges in DODMA to negative charges in the RNA is 1.8:2 to 0.8:2, more preferably 1.6:2 to 1:2, even more preferably 1.4:2 to 1.1:2 and even more preferably about 1.2:2. In an embodiment, the nanoparticles are lipoplexes comprising DODMA:Cholesterol:DOPE:PEGcerC16 in a molar ratio of 40:48:10:2.

In an embodiment, the particles can be lipoplexes comprising DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In an embodiment, the nanoparticles can be lipoplexes comprising DOTMA and Cholesterol in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In an embodiment, the nanoparticles can be lipoplexes comprising DOTAP and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

### Pharmaceutical Compositions and Modes of Administration

The multifunctional cells described herein, as well as the one or more RNA molecules encoding an immune receptor and two or more cytokines described herein may be administered in pharmaceutical compositions or medicaments and may be administered in the form of any suitable pharmaceutical composition.

For example, the cells and RNA molecules described herein may be administered in a pharmaceutical composition which may comprise a pharmaceutically acceptable carrier and may optionally comprise one or more adjuvants, stabilizers etc. In one embodiment, the pharmaceutical composition is for therapeutic or prophylactic treatments, e.g., for use in treating cancer.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severity of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in biocompatible polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Elsevier (A. Adejare edit. 2020).

Pharmaceutical carriers, excipients or diluents can be selected with regard to the intended route of administration and standard pharmaceutical practice.

In one embodiment, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical composition is formulated for systemic administration, e.g., for intravenous administration. In one embodiment in which the one or more RNA molecules are administered to the patient, the various modes of administration do not include intra-tumoral administration.

The term “co-administering” as used herein means a process whereby different compounds or compositions (e.g., RNA encoding an immune receptor and RNA encoding a cytokine) are administered to the same patient. The different compounds or compositions may be administered simultaneously, at essentially the same time, or sequentially.

The pharmaceutical compositions and products comprising one or more RNA molecules described herein may be provided as a frozen concentrate for solution for injection, e.g., at a concentration of 0.50 mg/mL. In one embodiment, for preparation of solution for injection, a drug product is thawed and diluted with isotonic sodium chloride solution (e.g., 0.9% NaCl, saline), e.g., by a one-step dilution process. In some embodiments, bacteriostatic sodium chloride solution (e.g., 0.9% NaCl, saline) cannot be used as a diluent. The concentration of the final solution for injection varies depending on the respective dose level to be administered.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The description (including the following examples) is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### DESCRIPTION OF THE FIGURES

FIG. 1:

Multifunctional murine NKG2D CAR T cells that co-express mIL12 and mIFNa2 have enhanced anti-tumor activity against syngeneic glioma cells in vitro.

A. Murine T cells were mock electroporated (ctrl.), or with two mRNAs encoding for mIL12 or mIFNa2 (Cyt), or an mRNA encoding for the NKG2D CAR (CAR) or all three mRNAs (CAR+Cyt). Subsequently, they were used as effector cells in co-culture with GL-261 glioma cells at different effector:target ratios. B. Modified T cells as described in A were co-cultured for 18 hours in a 2.5:1 effector:target ratio with GL-261 glioma cells and intracellular IFNg expression was determined by flow cytometry in CD4⁺ or CD8⁺ cells. C-D. Same setup as in A and B but CT-2A glioma cells were used as target cells. E-H. Same setup as in A-D, but the CAR was retrovirally transduced (CH). Data are presented as mean±SD (*/⁺p<0.05; **/⁺⁺p<0.01; *compared to ctrl., ⁺compared to CAR/CH).

FIG. 2:

Multifunctional mRNA-based NKG2D CAR T cells show anti-tumor activity in orthotopic immunocompetent murine glioma models upon intravenous and intratumoral administration.

A-B. Murine T cells were mock-electroporated (ctrl.) or transfected with mRNAs encoding mIL12 and mIFNa2 (Cyt), the NKG2D CAR (CAR), or all three proteins. Subsequently, the cells were labeled with CellBrite790 and 5×10⁶ cells were i.v. injected at days 4, 7, 10 and 13 after brain inoculation of GL-261 cells. The fluorescence signal from labeled T cells at the tumor site was detected by FMT at 12, 24 or 48 h after the first injection. One representative mouse per group is shown in A and quantification from 3 mice per group is shown in B. C. Same setup as in A but Kaplan Meier curves are shown. D. Same setup as in A, but the NKG2D CAR was retrovirally transduced (CH). E-F. GL-261 cells were implanted in naïve control mice or the contralateral hemisphere of the long-term surviving mice from C and D six months after the initial implantation. T2w MRI scans at day 15 after tumor implantation are shown. G-H. GL-261 (G) or CT-2A (H) tumor-bearing mice received two intratumoral injections of 2×10⁶ modified T cells as described in A at days 7 and 12 after tumor implantation. Kaplan Meier curves are shown and p values were calculated with log-rank test (*/⁺p<0.05; */⁺⁺p<0.01; * compared to ctrl, ⁺compared to CAR/CH).

FIG. 3:

Multifunctional mRNA-based NKG2D CAR T cells co-expressing mIL12 and mIFNa2 increase bystander T cells and pro-inflammatory cytokines in the tumor microenvironment and are less exhausted.

A-C. Murine CD45.1⁺ T cells were electroporated (ctrl.) or transfected with mRNAs encoding for mIL12 and mIFNa2 (Cyt), the NKG2D CAR (CAR), or all three transgenes and injected intratumorally into GL-261 glioma-bearing CD45.2⁺ mice at days 7 and 12 after tumor implantation. At day 14 after tumor implantation, tumor-infiltrating CD45.1⁺ (A, B) and CD45.2⁺ (C) immune cells were isolated and analyzed by flow cytometry. Data are presented as mean±SD (*/⁺p<0.05; **/⁺⁺p<0.01; * compared to ctrl., ⁺compared to CAR). D. Same experimental setup as in A-C, but cytokines were analyzed from dissociated tumor-bearing hemispheres by ELISA. The mean cytokine level is shown (*/⁺p<0.05; **/⁺⁺p<0.01; *compared to ctrl., ⁺compared to CAR). E-I. GL-261 glioma-bearing CD45.2⁺ mice were treated intratumorally at day 12 after tumor implantation with modified murine CD45.1⁺ T cells as indicated in A-C. 48 h later, FACS-sorted CD45.1⁺ T cells were characterized by RNA sequencing. Principal component analysis and expression level of the most abundant 30 genes are shown in E and F respectively. Gene enrichment scores in the different modified T cells are shown for genes associated with T cell activation or exhaustion (G), TGF-b response or IL10 response (H) or global T cells states comprising exhaustive, naïve, memory, activated or activated-dysfunctional cell states (I).

FIG. 4:

Human mRNA-based multifunctional CAR T cells co-expressing hIL12 and hIFNa2 show anti-tumor activity in complex glioblastoma patient samples with an intact microenvironment.

A. Scheme of ex vivo co-cultures of glioblastoma (GBM) patient samples with different mRNA-modified T cells. Surgically derived patient glioblastoma samples (n=10) were dissociated and co-cultured for 24 hours with mock-transfected T cells (ctrl.), hIL12 and hIFNa2 expressing T cells (Cyt), hNKG2D CAR T cells (CAR) or multifunctional CAR T cells co-expressing hNKG2D CAR as well as hIL12 and hIFNa2 (CAR+Cyt). B. Representative immunofluorescence images of one glioblastoma patient sample (Pat. 1) in the absence of modified T cells (left) and the presence of multifunctional CAR T cells co-expressing hIL12 and hIFNa2 (right). White arrows denote cancer cells. Scale bar, 30 μm. C. Representative single-cell crops of T cells classified by TNet according to their morphology as either activated (T_{ACT}) or non-activated (T_{CON}). Scale bar, 10 μm. D. Co-cultures from 10 dissociated glioblastoma patient samples and different modified T cells were set up as indicated in A. Number of glioma cells (bottom panel) and T cell activation states (top panel) in the different co-cultures for each patient were obtained on a single-cell resolution from the image analyses. The number of cancer cells is shown as box plots and the fraction of T cells adopting an activated morphology (T_{ACT} %) is shown as bar plots with mean±S.D. Black asterisks (all comparisons), green asterisks (compared to GBM only), red asterisks (compared to CAR+Cyt). *padj<0.05, **padj <0.01, ***padj <0.001.

FIG. 5:

Electroporation of murine T cells for mRNA transfection and cytolytic activity of multifunctional CAR T cells.
A. Murine T cells were electroporated with 2.5 μg mRNA encoding for ZSgreen at a voltage of 1600 mV applied in 3 pulses of 10 ms pulse width. The percentage of living cells and ZSgreen positive cells of all cells was determined by flow cytometry 24 h or 48 h after electroporation. B. Electroporation setup as in A, but increasing concentrations of mRNA were transfected and fluorescence intensity was detected 24 h later by flow cytometry. C and D. Electroporation as in A, but fluorescence intensity or percentage of fluorescent cells was determined for up to 6 days after electroporation by flow cytometry. E. Murine T cells were mock-electroporated with the parameter indicated in A or with mRNAs encoding either for the NKG2D CAR, or mIL12 and mIFNa2 or all three proteins. Transgene expression at 24 h was determined by flow cytometry. Data are presented as mean #SD (*p<0.05; compared to day 1 after electroporation). F and G. Murine T cells were mock electroporated (ctrl.), or with mRNAs encoding for either the NKG2D CAR (CAR), or the NKG2D CAR and mIL12 or mIFNa2 (CAR+IL12 and CAR+IFNa2), or all three mRNAs (CAR+IL12+IFNa2). Subsequently, they were used as effector cells in co-culture with GL-261 (F) or CT-2A (G) glioma cells at the indicated effector:target ratios.

FIG. 6:

Bodyweight measurements, therapeutic activity of modified T cells and target antigen expression.

A. Murine T cells were mock-electroporated (ctrl.) or transfected with mRNAs encoding mIL12 and mIFNa2 (Cyt), the NKG2D CAR (CAR), or all three proteins (CAR+Cyt). Subsequently, 5×10⁶ cells were i.v. injected at days 4, 7, 10 and 13 after brain inoculation of GL-261 cells. Bodyweight was assessed every other day. B. Same setup as in A but CT-2A glioma cells were used as a model and survival was monitored. Kaplan Meier curve is shown. C. Same setup as in A but treatment was administered at days 10, 13, and 16 after tumor implantation and survival was monitored. D. Same setup as in A, but the NKG2D CAR was retrovirally transduced (CH). E and F. GL-261 (C) or CT-2A (D) glioma-bearing mice were treated intratumorally at days 7 and 12 after tumor implantation with the different modified T cells as indicated in A. Mean±SD are shown. G. Same setup as in E but SMA-560 glioma-bearing mice were treated and survival was monitored. H. RAE-1 and MULT-1 were detected on GL-261, CT-2A and SMA-560 glioma cells by flow cytometry in vitro. Mean Fluorescence Intensity (MFI) is shown. I. Ex vivo glioma-bearing mouse brains comprising GL-261, CT-2A and SMA-560 cells were sectioned and stained for RAE-1 (green), CD45 (red) and DAPI (blue). Representative images are shown and quantification of RAE-1 staining intensity is shown on the right. Scale bar=100 μM.

FIG. 7:

Ex vivo flow cytometry analysis of spleen- and blood-derived immune cells upon intratumoral treatment of glioma-bearing mice with different modified T cells.
A. GL-261 glioma-bearing mice were treated intratumorally at days 7 and 12 after tumor implantation with 2×10⁶ murine T cells that were either mock-electroporated (ctrl.) or transfected with mRNAs encoding for mIL12 and mIFNa2 (Cyt), or the NKG2D CAR (CAR), or all three transgenes (CAR+Cyt). Spleens were isolated at day 14 after tumor implantation and analyzed by flow cytometry. B. Same setup as in A, but blood was analyzed. Mean±SD are shown.

FIG. 8:

Image-based analyses of co-cultures of cells from patient-derived glioblastoma samples with mRNA-modified T cells.
A. Thresholding strategy for marker-based classification of cells. Histograms and scatter plots of single-cell marker intensity distributions for S100B & CD3 (green, 488), NESTIN (yellow, PE), CD45 (red, APC). Data represents the entire set of cells imaged and analyzed. Single linear thresholds set marker for each were cellular as indicated. B. Convolutional neural network (CNN) test dataset accuracy. Accuracy of the original network (top) and the transfer-learned network on the manually curated test dataset of T_{ACT} and T_{CON} cells (bottom). The transfer-learned network (TNet) was subsequently used in all morphological analyses of T cells presented in this paper. C. Architecture of the original CNN from. Layers, where weights and biases were reset prior to the retraining of the transfer network, are highlighted in yellow. D. Patient tumors that were used for the image-based platform were stained for CD3⁺ T cells and the NKG2D ligands MICA and ULBP2. Staining intensity for each tumor is shown.

### EXAMPLE

### Material and Methods

### Culture of Glioma Cell Lines and Murine or Human T Cells

GL-261 cells were obtained from the National Cancer Institute (Frederick, Maryland, USA), SMA-560 cells were obtained from Dr. D. Bigner (Duke University Medical Center, Durham, North Carolina, USA) and CT-2A cells were purchased from Millipore (Temecula, California, USA). Glioma cell lines were cultured as described (Weiss et al., Clin Cancer Res. 2018; 24 (4): 882-895) and regularly tested negative for mycoplasma by PCR. Murine T cells derived from splenocytes or human T cells derived from peripheral blood mononuclear cells (PBMCs) were cultured in RPMI-1640 (Gibco Life Technologies, Waltham, Massachusetts, USA) supplemented with 10% FCS, 2 mM L-glutamine, (both purchased from Gibco), 100 U/ml penicillin-streptomycin (Sigma-Aldrich, St. Louis, Missouri, USA) and 50 U/ml murine or human IL2 (both PeproTech, Cranbury, New Jersey, USA). For murine T cells, 55 μM 2-mercaptoethanol (Gibco) was added.

In Vitro Transcription of mRNA

The murine and human NKG2D-based CAR constructs have been previously described (Baumeister S H et al., Cancer Immunol Res 2019; 7 (1): 100-12; Zhang T et al., Blood 2005; 106 (5): 1544-51). The mRNAs encoding for the murine or human NKG2D CAR or mIFNa2 were generated by in vitro transcription at the mRNA platform of Zurich as previously described (Tusup M et al., Chimia 2019; 73 (6): 391-94) and mRNAs encoding mIL12, hIL12 und hIFNa2 were obtained from BioNTech (Mainz, Germany). The functionality of synthetic mRNAs was confirmed by transfection of lymphocytes and subsequent detection of the respective protein by flow cytometry or ELISA.

### Generation of Multifunctional CAR T Cells

For the generation of murine multifunctional CAR T cells, splenocytes from C₅₇BL/6 mice were activated for 48 h using immobilized anti-CD3- and anti-CD28-antibodies (both BioXCell, Lebanon, New Hampshire, USA) at 1 mg/ml and 5 mg/ml respectively. Subsequently, between day 4 and day 7 following isolation, the cells were electroporated with mRNA encoding either the mNKG2D CAR (CAR) or mIL12 and mIFNa2 (Cyt) or all three mRNAs (CAR+Cyt) using 2.5 μg mRNA for the NKG2D CAR and 0.5 μg mRNA for each cytokine per million cells.

Human multifunctional CAR T cells were produced by activation of PBMCs using Dynabeads (Thermofisher, Waltham, Massachusetts, USA) for 72 h and electroporation of the mRNA as indicated between days 4 and 12 after activation.

Electroporation was performed using a NEON™ transfection system (Invitrogen, Carlsbad, California, USA) with electroporation parameters set to a voltage of 1600 mV and 3 pulses of 10 ms pulse width. Mock-electroporated cells served as control (ctrl.) and transfection of mRNA encoding for the fluorescent protein ZsGreen was used as a control for transfection efficiency. Following electroporation, the cells were kept in medium without antibiotics and used for experiments within a few hours. Stable retrovirally transduced NKG2D CAR T cells (CH) were generated as described (Zhang T et al., Blood 2005; 106 (5): 1544-51) and subsequently electroporated with mRNAs encoding for mIL12 and mIFNa2 (CH+Cyt) or mock electroporated on day 5.

### Antibodies and Flow Cytometry

For flow cytometry, the following monoclonal antibodies were used: anti-CD3-PerCP-Cy5.5, anti-CD4-BV650, anti-CD8-BV786, anti-CD11b-APC-Cy7, anti-CD45.1-AF488, anti-CD45.2-PE, anti-IFNg-BV421 and anti-NKp46-APC (BioLegend, San Diego, California, USA), anti-RAE-1-FITC and anti-MULT-1-PE (R&D Systems Europe, Abingdon, UK). To prevent unspecific staining all samples were preincubated with anti-mouse CD16/CD32 (BD Bioscience, Franklin Lakes, New Jersey, USA). Zombie Aqua™ (BioLegend) was used for viability staining and isotype-matched antibodies from Sigma-Aldrich served as controls.

Measurements were acquired on a FACSVerse or LSR II Fortessa and cell sorting was performed on a FACSAria III (all from BD, Franklin Lakes, New Jersey, USA). Data were processed in FlowJo (Tree Star, Stanford, California, USA). The isolation and analysis of tumor-infiltrating immune cells was performed as described (Weiss T et al., Clin Cancer Res 2018; 24 (4): 882-95).

### Immunofluorescence and Immunohistochemistry

Immunofluorescence and Immunohistochemistry was performed as described (Weiss T et al., Clin Cancer Res 2018; 24 (4): 882-95) using anti-CD3, anti-MICA, anti-ULBP2 from Sino Biological (Lucerna-Chem AG, Luzern, Switzerland) and anti-RAE1 from Novus Biologicals (Littleton, Colorado, USA). Images were analyzed in an unsupervised and blinded fashion using TMARKER, a software toolkit for histopathological staining estimation (Schuffler P J et al., J Pathol Inform 2013; 4 (Suppl): S2).

### In Vitro Cytotoxicity and IFNg Expression of CAR T Cells

Glioma cells as target cells were labeled with PKH26 (Sigma-Aldrich) and co-cultured with mRNA-based or virally transduced CAR T cells or respective control T cells with or without co-expression of mIL12 and mIFNa2 for 36-40 h at various effector:target ratios.

Target cell lysis was determined by flow cytometry as the percentage of death in the population of labeled target cells after subtraction of background lysis.

For the assessment of T cell-specific IFNg expression, a protein transport inhibitor cocktail (Invitrogen) was added after 18 h of co-culture and incubated for 6 h. Samples were subsequently stained for CD4, CD8 and intracellular IFNg and analyzed by flow cytometry.

### Pharmacoscopy

All studies including patient samples were approved by the Institutional Review Board and ethical committee (KEK-StV-Nr.19/08, BASEC number 2008-02002). Upon written informed consent, newly diagnosed glioblastoma samples were obtained from the Department of Neurosurgery at the University Hospital Zurich. Tumor dissociation was performed as described (Friebel E et al., Cell 2020; 181 (7): 1626-42.e20) and dissociated patient cells were seeded at 0.25-1.5×10⁴ cells/well into clear-bottom, tissue-culture treated, CellCarrier-384 Ultra Microplates (Perkin Elmer, Waltham, Massachusetts, USA). As indicated, 1×10⁴ control or mRNA-modified T cells were plated on top of patient cells in 25 μl/well of RPMI 1640 media supplemented with 10% FBS and cultured at 37° C., 5% CO₂ for 24 hours. Each condition had 6-8 corresponding replicate wells. Subsequently, cells were fixed with 4% PFA (Sigma-Aldrich), blocked with PBS containing 5% FBS and 0.1% Triton overnight and stained with the following antibodies: Alexa Fluor® 488 anti-S100 beta (Abcam), PE anti-Nestin (Biolegend), Alexa Fluor® 488 anti-CD3 (Biolegend), Alexa Fluor® 647 anti-CD45 (Biolegend) and DAPI (Biolegend).

Imaging of the 384 well plates was performed with an Opera Phenix automated spinning-disk confocal microscope at 20× magnification (Perkin Elmer). Single cells were segmented based on their nuclei (DAPI channel) using CellProfiler 2.2.0. Downstream image analysis was performed with MATLAB R2020a. Marker positive cell counts for each condition were derived based on a linear threshold of the histograms of each channel/marker intensity measurements across both plates. Marker positive cancer cell counts were averaged across each well/condition and compared between each treatment group.

### T Cell Morphology Deep Learning (TNet)

The original convolutional neural network (CNN) used for transfer learning was trained using a manually curated dataset of 16171 conventional T cells (T_{CON}) and 9599 activated T cells (T_{ACT}), utilizing a 39-layer CNN with an adapted ResNet architecture (He K et al., Deep Residual Learning for Image Recognition. 2016 27-30 Jun. 2016. p 770-78). A dataset of 50×50 pixel, 3-channel (DAPI, Brightfield, Alexa Fluor® 488) images of T cells was manually curated into T_{CON} and T_{ACT} morphological classes, generating a total of 5564 T_{CON} and 4269 T_{ACT} cells. Curated images of CAR T cells were rescaled to 48×48 pixels before training, and training and validation datasets were split 4:1 to evaluate overfitting of the CNN both during and after training. Before CNN training, the weights and biases of the original network were transferred, except for the last convolutional layer and final fully connected layer, which were reset and randomly initialized. To improve the learning rate of the new layers compared with the transferred layers, the weight and bias learn rate factors of the new layers were set to 10. The network was trained for 20 epochs implementing the adaptive learning rate optimization ‘ADAM’, with an initial learning rate of 0.001 which was lowered with a factor of 0.1 every 5 epochs. A mini batch size of 256 images and L² regularization with 0.001 was applied. In each training iteration, images were randomly rotated by 45-degrees and mirrored vertically or horizontally per iteration to limit orientation biases towards cellular features. Performance of the network was assessed with a separate test dataset composed of 854 T_{ACT} and 1113 T_{CON} cells.

### RNA Sequencing

CD45.1⁺ cells were isolated from tumor-bearing hemispheres by FACS sorting and immediately fixed in Trizol reagent (Invitrogen). Subsequently, extraction of total RNA was performed using the RNeasy MinElute™ Cleanup Kit (Qiagen, Hilden, Germany). Library prep was performed using the RNA Prep with Enrichment (L) Tagmentation′ Kit (Ilumina, San Diego, USA), following manufacturer's instructions. Resulting pooled library was sequenced with a NextSeq 500/550 High Output Kit v2.5 (75 Cycles) at a final concentration of 1.8 pM. Generated Fastq files were aligned using the STAR aligner.

### Mice and Animal Experiments

All experiments were done following the guidelines of the Swiss federal law on animal protection and approved by the cantonal veterinary office (ZH073-2018). C57BL/6^{CD45.2} mice were purchased from Charles River Laboratories (Sulzfeld, Germany). C57BL/6^{CD45.1} were bred in pathogen-free facilities at the University of Zurich. For all experiments, mice of 6 to 12 weeks of age were used and stereotactic tumor implantation of 2×10⁴ GL-261 or 75×10⁴ CT-2A cells and monitoring of mice has been described (Weiss T et al., Clin Cancer Res 2018; 24 (4): 882-95).

### Multicytokine Enzyme-Linked Immunosorbent Assay (ELISA)

Tumor-bearing hemispheres were dissociated in 2 ml PBS. Subsequently, cytokines were measured in a 100 μl aliquot of the supernatant using a multi-analyte ELISArray Kit (Qiagen, Hilden, Germany).

### Magnetic Resonance Imaging (MRI)

On day 15 after tumor implantation coronal T2-weighted image sequences of the mouse brains were recorded using a 4.7 T MRI and Paravision 6.0 (Bruker Biospin, Ettlingen, Germany).

### Fluorescence Molecular Tomography (FMT)

Murine T cells were labeled with CellBrite™ NIR790 (Biotium, Fremont, California, USA), electroporated and administered to tumor-bearing mice as indicated. For the in vivo tracking of adoptively transferred cells, we used the 790 nm laser channel of a FMT2500 system and images were analyzed using TrueQuant 3.1 (PerkinElmer).

### Statistical Analysis

Data are presented as means+/−SD. Where not indicated differently, experiments were repeated at least three times. GraphPad Prism (La Jolla, CA, USA) was employed for statistical analysis using two-way ANOVA and correcting for multiple comparisons using the two-stage step-up method of Benjamini, Krieger and Yekutieli. Kaplan Meier survival analysis was performed to assess survival differences among the treatment groups and p values were calculated with the log-rank test. RNA-sequencing reads were normalized and adjusted for differential gene expression using DESeq. Significance was concluded at *p<0.05 and **p<0.01 throughout all figures.

### Results

Multifunctional NKG2D CAR T Cells Co-Expressing mIL12 and mIFNa2 Exert Enhanced Anti-Tumor Activity Against Syngeneic Murine Glioma Cell Lines In Vitro

We first established the optimal parameters for mRNA electroporation into mouse T cells using mRNA encoding for the fluorescent protein ZsGreen. A voltage of 1600 mV applied in 3 pulses of 10 ms pulse width led to transfection efficiency with preserved viability of cells (FIG. 5A). The transgene expression was titratable with increasing concentrations of mRNA (FIG. 5B) and stable for up to five days upon electroporation (FIGS. 5C and D). We further showed that simultaneous transfection of multiple mRNAs did not impair transfection efficiency (FIG. 5E).

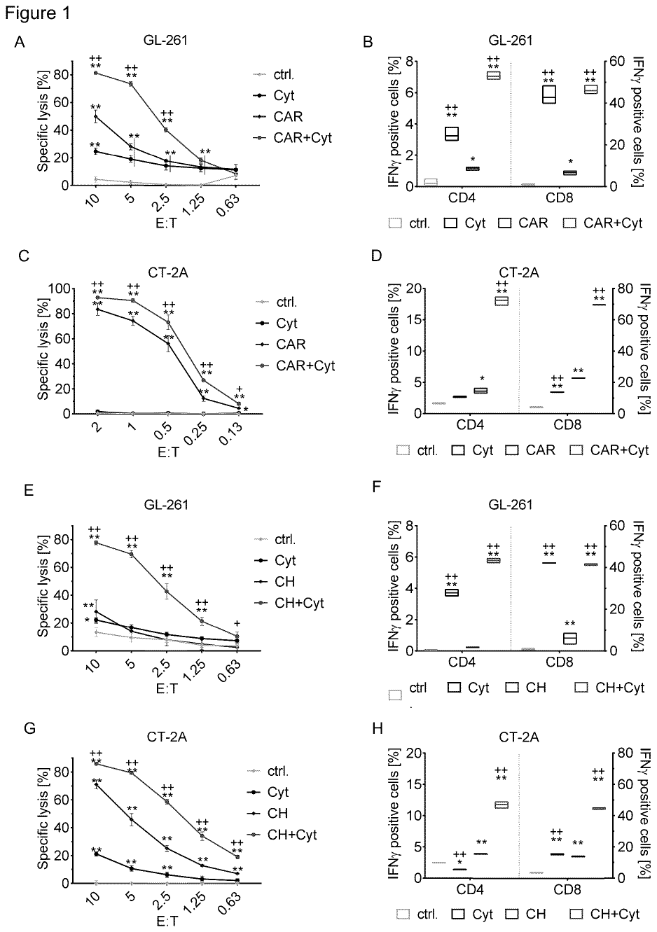

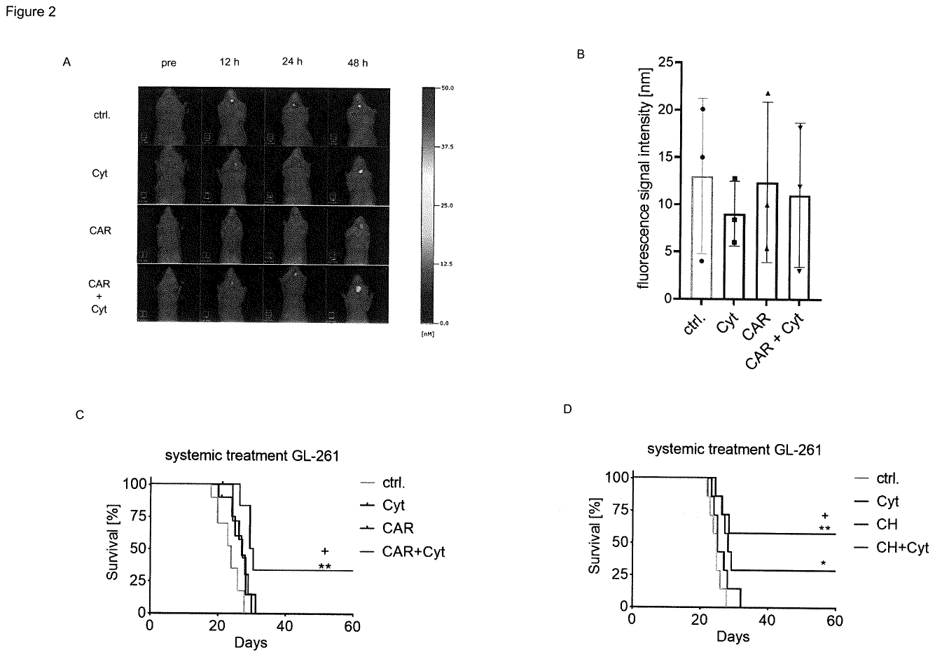

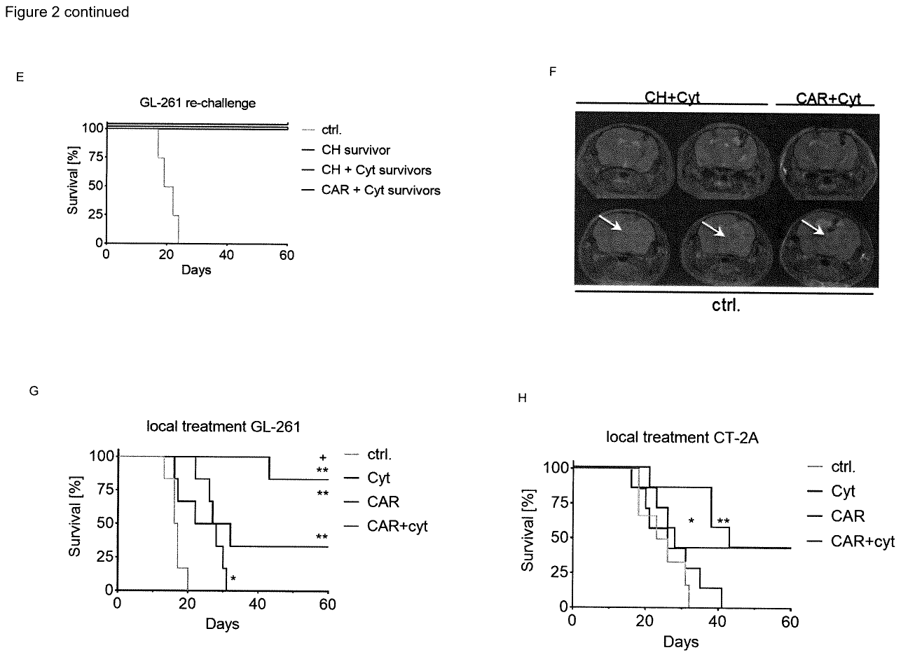

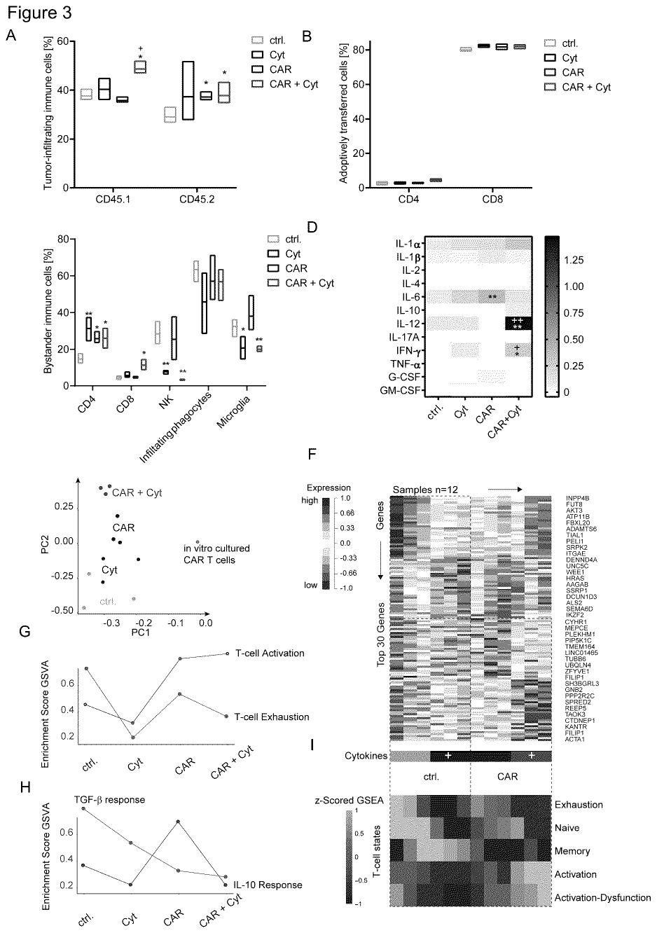

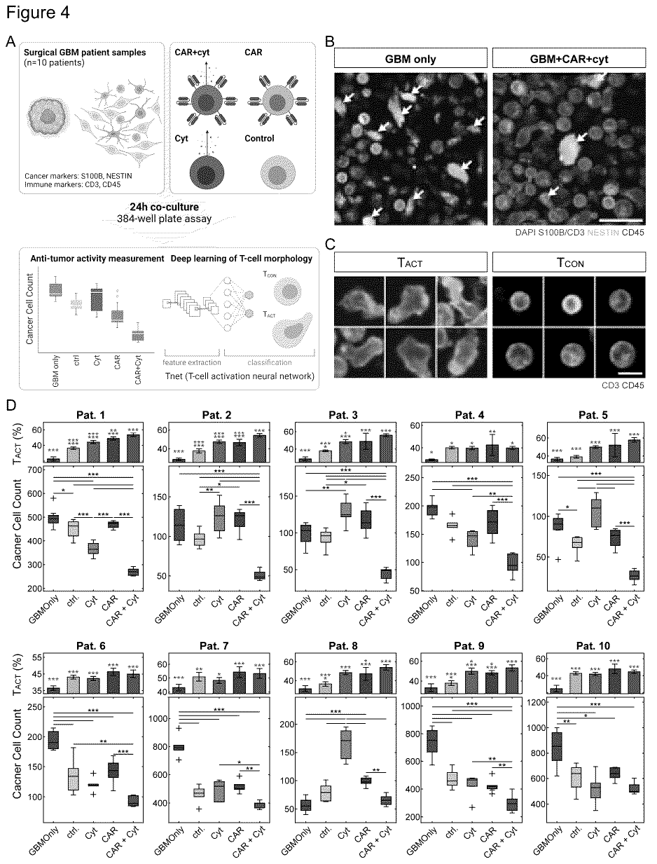

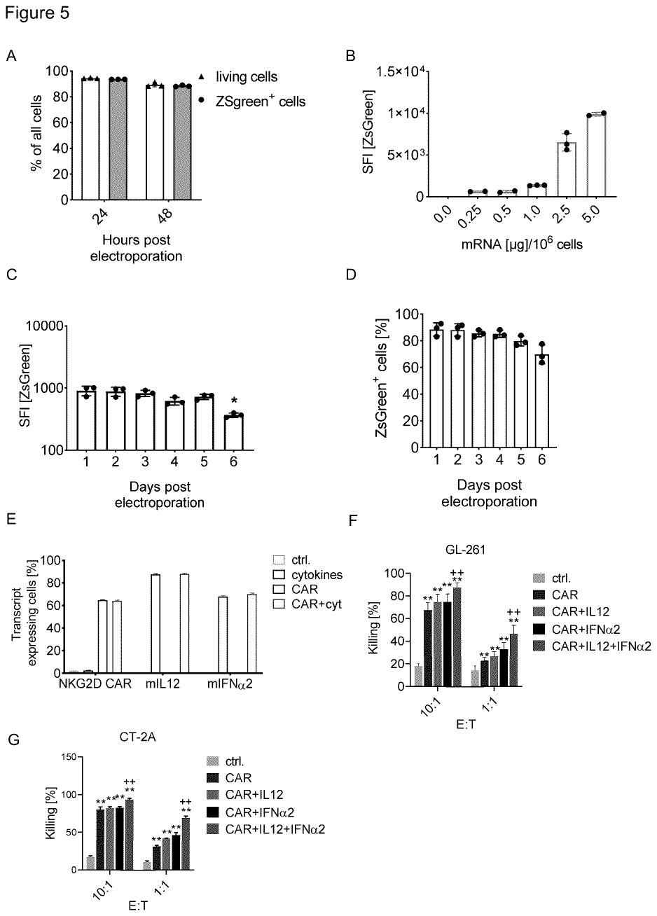

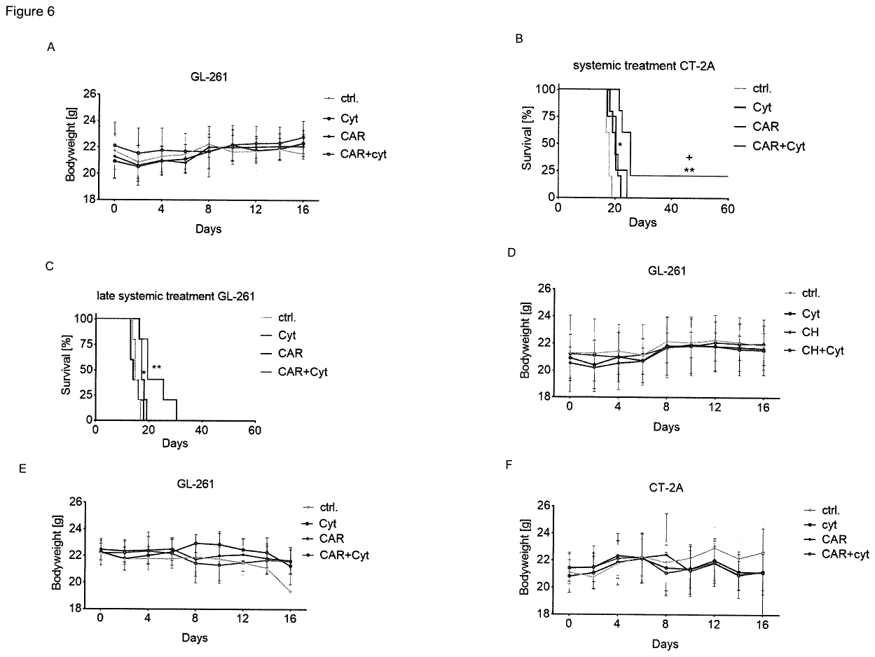

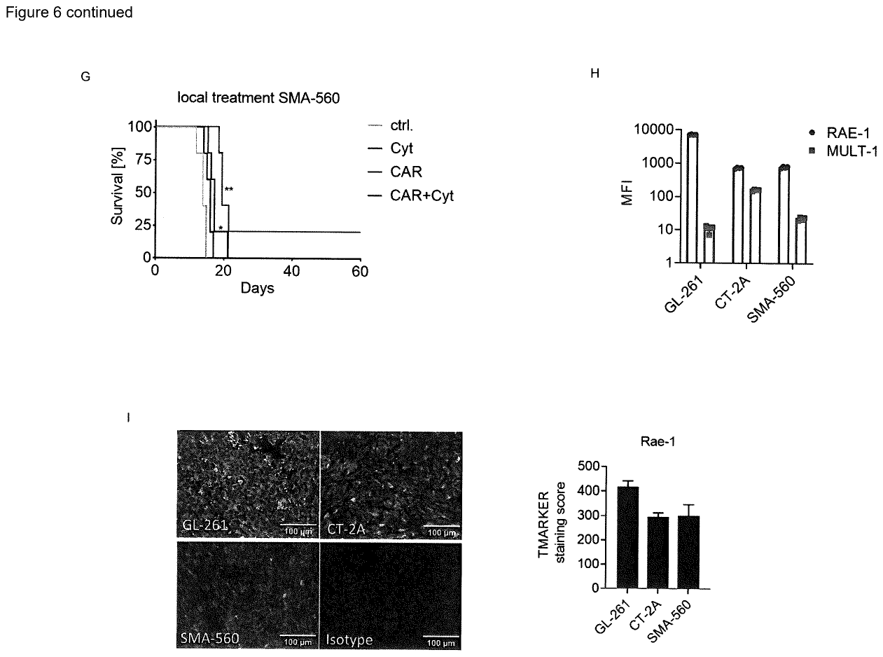

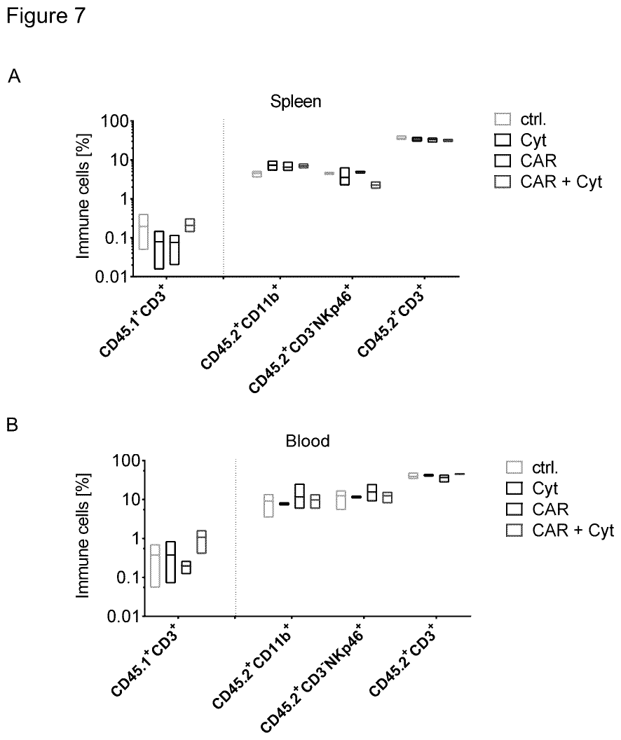

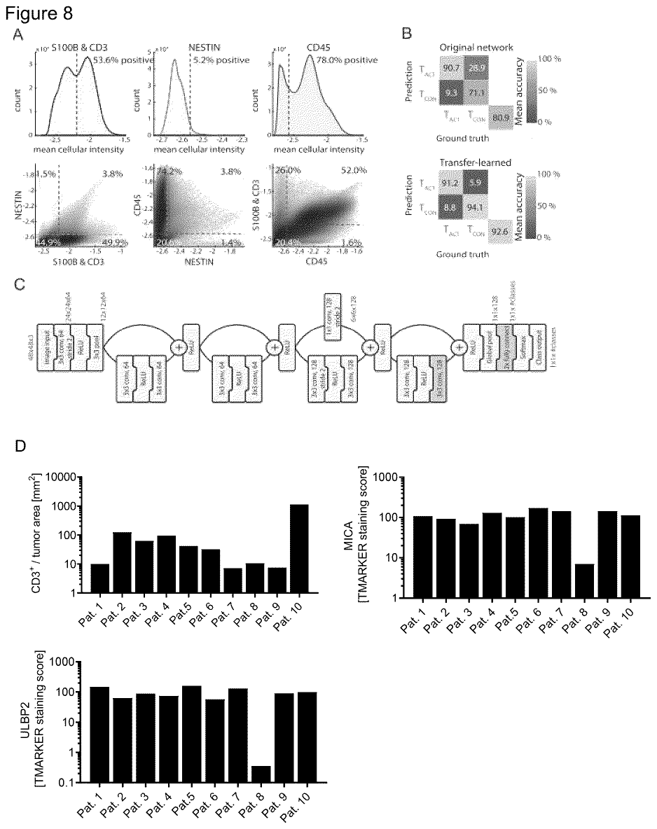

We electroporated murine T cells with mRNA to express either the pro-inflammatory cytokines mIL12 and mIFNa2 (Cyt) or the NKG2D CAR (CAR) or all three transgenes (CAR+Cyt) and used these cells as effector cells in co-cultures with the murine glioma cell lines GL-261 or CT-2A. NKG2D CAR T cells that co-expressed mIL12 and mIFNa2 had the highest cytolytic activity (FIGS. 1A and C). The co-expression of mIL12 and mIFNa2 in addition to the CAR was also superior to the combination of the CAR with either cytokine alone (FIGS. 5F and G). In subsequent experiments we focused on the co-expression of all three transgenes. Co-expression of the CAR and the cytokines also led to the highest IFNγ expression in CD4 T cells against both GL-261 and CT-2A glioma cells (FIG. 1B, D), whereas for CD8 T cells the effect differed according to the target cell line: Against CT-2A the CAR and cytokine-expressing CD8 T cells had the highest IFNγ expression, whereas against GL-261 already the cytokines alone increased the level of IFNγ to a similar level compared to the co-expression of the CAR and the cytokines (FIGS. 1B, D).

Many CAR T approaches rely on the viral transduction of a CAR construct. We generated stable NKG2D CAR T cells by retroviral transduction (CH), which were subsequently electroporated with mRNAs encoding for mIL12 and mIFNa2. This further improved their cytolytic activity and intracellular IFNg expression in co-culture with GL-261 or CT-2A glioma cells (FIG. 1E-H).

Multifunctional NKG2D CAR T Cells Co-Expressing mIL12 and mIFNa2 have Anti-Tumor Activity in Immunocompetent Orthotopic Glioma Mouse Models Upon Intravenous and Local Administration

We first characterized the tumor homing of mRNA-based murine T cells expressing either mIL12 and mIFNa2 or the NKG2D CAR or all three proteins upon intravenous administration. FMT imaging demonstrated a similar accumulation of the different modified T cells in the tumor upon i.v. injection in immunocompetent orthotopic GL-261 glioma-bearing mice (FIGS. 2A and B).

Intravenously administered multifunctional mRNA-based NKG2D CAR T cells that co-expressed mIL12 and mIFNa2 conferred a survival benefit and led to a fraction of long-term surviving mice whereas mRNA-modified T cells that expressed either the cytokines or the NKG2D CAR did not confer a survival benefit (FIG. 2C). The treatment was well tolerated and we did not observe signs of toxicity at the level of mouse behavior or bodyweight (FIG. 6A). The anti-glioma activity of intravenously administered multifunctional cytokine-expressing CAR T cells could also be confirmed in the less immunogenic CT-2A glioma model (FIG. 6B) and we also observed a significant survival benefit (FIG. 6C). Furthermore, the co-electroporation of mRNAs encoding for mIL12 and mIFNa2 also improved the anti-tumor activity of intravenously administered viral vector-based NKG2D CAR T cells and increased the number of long-term surviving mice (FIG. 2D) without signs of toxicity at the level of bodyweight measurements (FIG. 6D). Long-term surviving mice did not develop tumors upon re-challenge with GL-261 glioma cells into the contralateral hemisphere six months after the initial treatment, showing a long-lasting anti-tumor effect (FIGS. 2E and F). For all subsequent experiments, we used mRNA-modified T cells due to their potential for translation because of faster manufacturing processes and a better safety profile compared to virally transduced T cells due to the transient nature. We also used local intra-tumoral administration of multifunctional mRNA-based CAR T cells. This was also well-tolerated in orthotopic GL-261 glioma-bearing mice without signs of toxicity at the level of behavior and bodyweight (FIG. 6E) and further improved the therapeutic efficacy compared to intravenous administration. Two intra-tumoral injections of mRNA-based multifunctional NKG2D CAR T cells that co-expressed mIL12 and mIFNa2 cured 84% of GL-261 glioma-bearing mice (FIG. 2G). The promising anti-glioma activity and tolerability of intratumorally administered mRNA-based mIL12- and mIFNa2-expressing NKG2D CAR T cells was also confirmed in two additional immunocompetent, orthoptic glioma models using CT-2A or SMA-560 glioma cells (FIG. 2H, FIG. 6F-G). These models are less immunogenic and express also less NKG2D ligands compared to GL-261 (FIGS. 6H and I).

Multifunctional mRNA-Based NKG2D CAR T Cells Co-Expressing mIL12 and mIFNa2 Increase Bystander T Cells and Pro-Inflammatory Cytokines in the Tumor Microenvironment and are Less Exhausted

We characterized adoptively transferred mRNA-modified T cells, bystander tumor-infiltrating immune cells and cytokines in the tumor microenvironment upon local intra-tumoral treatment in immunocompetent orthotopic glioma-bearing mice. Co-expression of mIL12 and mIFNa2 in addition to the NKG2D CAR led to the strongest increase in the abundance of adoptively transferred CD45.1⁺ T cells within the tumor microenvironment. Furthermore, multifunctional cytokine-expressing CAR T cells increased the abundance of CD45.2⁺bystander immune cells within the tumor microenvironment (FIG. 3A). The vast majority of the adoptively transferred cells were CD8 positive, independent of the modification (FIG. 3B). Multifunctional cytokine- and CAR-expressing T cells increased the fraction of both tumor-infiltrating CD45.2⁺CD4 and CD8 T cells, whereas the cytokine- or CAR-only expressing T cells mainly increased the fraction of bystander CD4 T cells (FIG. 3C). Ex vivo cytokine profiling demonstrated increased IL12 and IFNg levels in the tumor microenvironment upon treatment with multifunctional CAR T cells, whereas CAR-only expressing T cells mainly led to an upregulation of IL6 (FIG. 3D). We did not detect differences in immune cell populations in the blood and spleen suggesting a locally confined effect at the tumor site (FIG. 7). We furthermore FACS-sorted tumor-infiltrating CD45.1⁺ and performed RNA sequencing. Principal component analysis showed a clear separation of the four different transfected T cell populations (FIG. 3E) and we identified distinct gene expression signatures based on the 30 most abundant genes (FIG. 3F). We performed gene set enrichment analysis using previously reported gene sets associated with T cells exhaustion or activation, respectively (Singer M et al., Cell 2016; 166 (6): 1500-11.e9). This revealed that genes associated with T cell exhaustion were predominantly enriched in control T cells and T cells that expressed the CAR only whereas genes that are associated with T cell activation showed the highest enrichment in T cells that co-expressed the CAR and mIL12 and mIFNα2 (FIG. 3G).

In line with these findings, genes involved in the T cell response to the immunosuppressive cytokine TGF-β, which has been associated with T cell exhaustion in previous studies (Gunderson A J et al., Nature communications 2020; 11 (1): 1749), showed the lowest expression in multifunctional CAR T cells co-expressing mIL12 and mIFNα2. Genes associated with an immunosuppressive IL10 response were enriched in CAR T cells but not upon co-expression of mIL-12 and mIFNα2 (FIG. 3H). Accordingly, multifunctional CAR T cells co-expressing mIL12 and mIFNα2 displayed the strongest overall T cell activation state (FIG. 3I).

Human mRNA-Based Multifunctional CAR T Cells Co-Expressing hIL12 and hIFNa2 have Anti-Tumor Activity in Glioblastoma Patient Samples with a Complex Microenvironment

We generated PBMC-derived mRNA-modified T cells encoding either the human NKG2D CAR or hIL12 and hIFNa2 or all three proteins, and showed the translational potential of multifunctional mRNA-based CAR T cells for the treatment of glioblastoma. We obtained tissue samples directly from surgery of newly diagnosed glioblastoma patients, dissociated the samples and exposed the complex cell mixtures comprising glioma cells and cells from the tumor microenvironment to the different modified T cells. After 24 h, we used an image-based platform (Snijder B et al., The Lancet Haematology 2017; 4 (12): e595-e606) to determine the anti-tumor activity of the different engineered T cells on a single cell level (FIG. 4A). Markers associated with glial lineage and stemness (S100B, NESTIN) were used to identify S100B+CD45 or NESTIN+CD45 cancer cells whereas CD45 was used as a pan-immune cell counterstain. T cells were identified by the co-expression of CD3 and CD45 (FIG. 4B, FIG. 8A-C). This single-cell phenotypic read-out from 10 glioblastoma samples demonstrated that despite interpatient heterogeneity, multifunctional mRNA-based CAR T cells co-expressing hIL12 and hIFNa2 most efficiently reduced the number of cancer cells (FIG. 4B, D). In contrast, T cells that only expressed either the NKG2D CAR or the pro-inflammatory cytokines did not consistently reduce the number of glioma cells. In all patients except for patient 8, which showed very low target antigen expression (FIG. 8D), multifunctional CAR T cells reduced the number of glioma cells by around 50%. To determine whether specific T cell phenotypes were associated with increased anti-glioma activity, we assessed T cell activation through deep learning-based morphological profiling (FIGS. 4C and D). We trained a convolutional neural network (TNet) to classify T cells as either activated (T_{ACT}) or non-activated (T_{CON}), based on whether they displayed rounded or polarized morphologies. In 6 out of 10 patients (60%), T cells that co-expressed the CAR and hIL12/hIFNα2 had the highest fraction of T_{ACT} cells; 4 of which were significantly higher than the NKG2D CAR T cells alone, showing higher levels of activation.

We claim:

1. A multifunctional cell recombinantly expressing an immune receptor and two or more cytokines, wherein the cell comprises one or more exogenous RNA molecules encoding the immune receptor and the two or more cytokines.

2. The multifunctional cell according to claim 1, wherein the cell does not contain any exogenous DNA molecules encoding the immune receptor and/or the two or more cytokines.

3. The multifunctional cell according to claim 1 or 2, wherein the one or more exogenous RNA molecules encodes three or more cytokines.

4. The multifunctional cell according to any one of claims 1 to 3, wherein the immune receptor is encoded by a separate exogenous RNA molecule from the two or more cytokines.

5. The multifunctional cell according to any one of claims 1 to 4, wherein each of the two or more cytokines is encoded by separate exogenous RNA molecules.

6. The multifunctional cell according to any one of claims 1 to 5, wherein the immune receptor and the two or more cytokines are encoded by a single RNA molecule.

7. The multifunctional cell according to any one of claims 1 to 6, wherein the one or more exogenous RNA molecules is an in vitro transcribed RNA molecule.

8. The multifunctional cell according to any one of claims 1 to 7, wherein the one or more exogenous RNA molecules is a synthetic RNA molecule.

9. The multifunctional cell according to any one of claims 1 to 8, wherein the one or more exogenous RNA molecules is not produced by transcription from DNA in the multifunctional cell.

10. The multifunctional cell according to any one of claims 1 to 9, wherein the cell is obtained from peripheral blood, bone marrow, spleen, tumor infiltrating lymphocytes or from a cell line.

11. The multifunctional cell according to any one of claims 1 to 10, wherein the cell is a peripheral blood mononuclear cell, a bone marrow cell, a lymphocyte, a splenocyte, or a T-cell.

12. The multifunctional cell according to any one of claims 1 to 11 which is a cytotoxic cell.

13. The multifunctional cell according to any one of claims 1 to 14 which is a CD8⁺ T cell.

14. The multifunctional cell according to any one of claims 1 to 13, wherein the immune receptor is a chimeric antigen receptor (CAR).

15. The multifunctional cell according to any one of claims 1 to 14, wherein the immune receptor is a T-cell receptor (TCR).

16. The multifunctional cell according to any one of claims 1 to 15, wherein the two or more cytokines are selected from two or more of the following cytokines: interleukin-2, interleukin-7, interleukin-10, interleukin-12, interleukin-15, interleukin-18 and interferon-α (IFN-α).

17. The multifunctional cell according to claim 16, wherein IFN-α is a subtype of IFN-α, for example, IFN-α1, IFN-α2, IFN-α8, IFN-α10, IFN-α14 or IFN-α21.

18. The multifunctional cell according to any one of claims 1 to 17, wherein the one or more exogenous RNA molecules is a linear mRNA, a circular mRNA, a self-replicating RNA, or a mixture of the different formats.

19. The multifunctional cell according to any one of claims 1 to 18, wherein each exogenous RNA molecule is a linear mRNA.

20. The multifunctional cell according to any one of claims 1 to 19, wherein the two or more cytokines are interleukin-12 and interferon-α2.

21. The multifunctional cell according to any one of claims 1 to 20, wherein the immune receptor binds an antigen expressed on a tumor cell.

22. The multifunctional cell according to claim 21, wherein the tumor cell is a glioblastoma.

23. The multifunctional cell according to claim 21 or 22, wherein the antigen expressed on the tumor cell is a ligand of NKG2D receptor.

24. A multifunctional cell recombinantly expressing an immune receptor and two or more cytokines for use in a method of treating a disease or disorder in a patient characterized by the expression of an antigen to which the immune receptor binds, wherein the cell comprises one or more exogenous RNA molecules encoding the immune receptor and the two or more cytokines, said method comprising administering the multifunctional cell to the patient, preferably wherein the patient is a human.

25. The multifunctional cell for use according to claim 24, wherein the cell does not contain any exogenous DNA molecules encoding the immune receptor and/or the two or more cytokines.

26. The multifunctional cell for use according to claim 24 or 25, wherein the one or more exogenous RNA molecules encodes three or more cytokines.

27. The multifunctional cell for use according to any one of claims 24 to 26, wherein the immune receptor is encoded by a separate exogenous RNA molecule from the two or more cytokines.

28. The multifunctional cell for use according to any one of claims 24 to 27, wherein each of the two or more cytokines is encoded by separate exogenous RNA molecules.

29. The multifunctional cell for use according to any one of claims 24 to 28, wherein the immune receptor and the two or more cytokines are encoded by a single RNA molecule.

30. The multifunctional cell for use according to any one of claims 24 to 29, wherein the one or more exogenous RNA molecules is an in vitro transcribed RNA molecule.

31. The multifunctional cell for use according to any one of claims 24 to 30, wherein the one or more exogenous RNA molecules is a synthetic RNA molecule.

32. The multifunctional cell for use according to any one of claims 24 to 31, wherein the one or more exogenous RNA molecules is not produced by transcription from DNA in the multifunctional cell.

33. The multifunctional cell for use according to any one of claims 24 to 32, wherein the cell is obtained from peripheral blood, bone marrow, tumor infiltrating lymphocytes, spleen, or from a cell line.

34. The multifunctional cell for use according to any one of claims 24 to 33, wherein the cell is a peripheral blood mononuclear cell, a bone marrow cell, a lymphocyte, a splenocyte, or a T-cell.

35. The multifunctional cell for use according to any one of claims 24 to 34 which is a cytotoxic cell.

36. The multifunctional cell for use according to any one of claims 24 to 35 which is a CD8+ T cell.

37. The multifunctional cell for use according to any one of claims 24 to 36, wherein the immune receptor is a chimeric antigen receptor (CAR).

38. The multifunctional cell for use according to any one of claims 24 to 37, wherein the immune receptor is a T-cell receptor (TCR).

39. The multifunctional cell for use according to any one of claims 24 to 38, wherein the two or more cytokines are selected from two or more of the following cytokines: interleukin-2, interleukin-7, interleukin-10, interleukin-12, interleukin-15, interleukin-18 and interferon-α (IFN-α).

40. The multifunctional cell according to claim 39, wherein IFN-α is a subtype of IFN-α, for example, IFN-α1, IFN-α2, IFN-α8, IFN-α10, IFN-α14 or IFN-α21.

41. The multifunctional cell for use according to any one of claims 24 to 40, wherein the one or more exogenous RNA molecules is a linear mRNA, a circular mRNA, a self-replicating RNA, or a mixture of the different formats.

42. The multifunctional cell for use according to any one of claims 24 to 41, wherein each exogenous RNA molecule is a linear mRNA.

43. The multifunctional cell for use according to any one of claims 24 to 42, wherein the two or more cytokines are interleukin-12 and interferon-α2.

44. The multifunctional cell for use according to any one of claims 24 to 43, wherein the immune receptor binds an antigen expressed on a tumor cell.

45. The multifunctional cell for use according to any one of claims 24 to 44, wherein, wherein the disease or disorder is cancer.

46. The multifunctional cell for use according to claim 45, wherein the cancer is glioblastoma.

47. The multifunctional cell for use according to any one of claims 24 to 46, wherein the cell administered to the patient is autologous or allogeneic to the patient.

48. The multifunctional cell according to any one of claims 1 to 23 or the multifunctional cell for use according to any one of claims 24 to 47, wherein the immune cell comprises a further genetic modification.

49. The multifunctional cell or the multifunctional cell for use according to claim 48, wherein the genetic modification comprises (i) disrupting the expression of the endogenous T cell receptor (TCR) such that the TCR is expressed at reduced levels and/or (ii) disrupting the expression of the HLA/MHC complex such that the complex is expressed on the cell surface at reduced levels.

50. The multifunctional cell or the multifunctional cell for use according to claim 49, wherein the reduced level of expression is no more than 35%, 30%, 25%, 20%, 15%, 10%, 5%, 4%, 3%, 2%, or 1% of the wild-type level of expression.

51. The multifunctional cell or the multifunctional cell for use according to claim 49, wherein the endogenous TCR and/or the HLA/MHC complex is not detectably expressed on the surface of the immune cell.

52. The multifunctional cell or the multifunctional cell for use according to any one of claims 49 to 51, wherein the expression of the endogenous TCR and/or the HLA/MHC complex on the cell surface is determined using a FACS assay.

53. A cytotoxic T cell expressing a chimeric antigen receptor (CAR) capable of binding to a NKG2D ligand, interleukin-12, and interferon-α2 for use in a method of treating glioblastoma, wherein the cytotoxic T cell comprises three exogenous mRNA molecules, the first encoding the CAR, the second encoding interleukin-12, and the third encoding interferon-α2.

54. The cytotoxic T cell according to claim 53 which does not comprise any exogenous DNA sequences encoding the CAR, interleukin-12 and/or interferon-α2.

55. A method for producing a multifunctional cell expressing an immune receptor and two or more cytokines, said method comprising transfecting a mononuclear cell with one or more exogenous RNA molecules encoding the immune receptor and the two or more cytokines to produce a multifunctional cell expressing the immune receptor and the two or more cytokines.

56. The method according to claim 55, wherein the one or more exogenous RNA molecules is a linear mRNA, a circular mRNA, a self-replicating RNA, or a mixture of the different formats.

57. The method according to claim 55 or 56, wherein the immune receptor and the two or more cytokines are encoded by a single exogenous RNA molecule.

58. The method according to claim 55 or 56, wherein the two or more cytokines are encoded by a first exogenous RNA molecule and the immune receptor is encoded by a second exogenous RNA molecule.

59. The method according to claim 55 or 56, wherein each of the two or more cytokines is encoded by separate exogenous RNA molecules.

60. The method according to any one of claims 55 to 59, wherein the one or more exogenous RNA molecules is complexed with a lipid particle or is complexed with a polymer.

61. The method according to any one of claims 55 to 60, wherein transfection of the one or more exogenous RNA molecules is by a method selected from the group consisting of electroporation, lipid-mediated transfection, calcium phosphate transfection, targeted liposomes, polymer-mediated transfection, particle mediated delivery, microbubble-assisted focused ultrasound (FUS).

62. The method according to claim 61, wherein transfection is carried out by electroporation.

63. The method according to claim 61, wherein transfection is carried out by lipid-mediated transfection.

64. The method according to any one of claims 55 to 63, wherein the multifunctional cell is a cytotoxic cell.

65. The method according to any one of claims 55 to 64, wherein the method further comprises (i) disrupting the expression of the endogenous T cell receptor (TCR) such that the TCR is expressed on the cell surface at reduced levels and/or (ii) disrupting the expression of the HLA/MHC complex such that the complex is expressed on the cell surface at reduced levels.

66. The method according to claim 65, wherein the endogenous TCR and/or the HLA/MHC complex is not detectably expressed on the surface of the multifunctional cell.

67. A multifunctional cell, preferably a cytotoxic T cell, produced by the method according to any one of claims 55 to 66.

68. The multifunctional cell according to claim 67 for use in a method of treating a disease or disorder in a patient characterized by expression of an antigen to which the immune receptor binds, said method comprising administering to the patient the multifunctional cell.

69. The multifunctional cell for use according to claim 68, wherein the disease or disorder is cancer.

70. The multifunctional cell for use according to claim 69, wherein the cancer is glioblastoma.

71. A method of treating a disease or disorder in a patient comprising administering to the patient a multifunctional cell recombinantly expressing an immune receptor and two or more cytokines, wherein the disease or disorder is characterized by the expression of an antigen to which the immune receptor binds, wherein the cell comprises one or more exogenous RNA molecules encoding the immune receptor and the two or more cytokines, preferably wherein the patient is a human.

72. One or more exogenous RNA molecules encoding an immune receptor and two or more cytokines for use in a method of treating a disease or disorder in a patient, said method comprising administering to the patient the one or more exogenous RNA molecules, wherein the disease or disorder is characterized by the expression of an antigen to which the immune receptor binds, preferably wherein the patient is a human

73. A method of treating cancer in a patient comprising administering to the patient one or more exogenous RNA molecules encoding an immune receptor and two or more cytokines, wherein the cancer is characterized by the expression of a cancer antigen to which the immune receptor binds, preferably wherein the patient is a human.


SEQUENCE LISTING (3 sequences)

SEQ ID NO: 1 - RNA, 47 nt, synthetic construct
  source 1..47: mol_type = other RNA
aactagtattcttctggtccccacagactcagagagaacccgccacc

SEQ ID NO: 2 - RNA, 278 nt, synthetic construct
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 3 - DNA, 110 bp, synthetic construct
  source 1..110: mol_type = other DNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa